SEQUENCE LISTING (74 sequences)

SEQ ID NO: 1 - protein, 282 aa, synthetic construct [sequence omitted]

SEQ ID NO: 2 - protein, 305 aa, synthetic construct
  source 1..305: note = Human DNAse1L3
MSRELAPLLLLLLSIHSALAMRICSFNVRSFGESKQEDKNAMDVIVKVIKRCDIILVMEIKDSNNRICPILMEKLNRNSRRGITYNYVISSRLGRNTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRS

SEQ ID NO: 3 - protein, 21 aa, synthetic construct
  source 1..21: note = X1 peptide sequence
RAFTNNRKSVSLKKRKKGNRS

SEQ ID NO: 4 - protein, 227 aa, synthetic construct
  source 1..227: note = hIgG1 Fc domain, Fc human
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 10 aa, synthetic construct
  source 1..10: note = Linker
GGGGSGGGGS

SEQ ID NO: 6 - protein, 25 aa, synthetic construct
  source 1..25: note = Linker
ELKTPLGDTTHTXPRZPAPELLGGP

SEQ ID NO: 7 - protein, 511 aa, synthetic construct
  source 1..511: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 511 aa, synthetic construct
  source 1..511: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 511 aa, synthetic construct
  source 1..511: note = DNAse 1 Construct
MRGMKLLGALLALAALLQGAVSLKIAAFNIQTFGRTKMSNATLVSYIVQILSRYDIALVQEVRDSHLTAVGKLLDNLNQDAPDTYHYNVSEPLGRNSYKERYLFVYRPNQTSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYNLSNQTAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 553 aa, synthetic construct
  source 1..553: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 11 - protein, 532 aa, synthetic construct
  source 1..532: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 12 - protein, 532 aa, synthetic construct
  source 1..532: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 519 aa, synthetic construct
  source 1..519: note = DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 534 aa, synthetic construct
  source 1..534: note = DNAse1 Construct
  VAR_SEQ 295: note = X is Cys, Gly, or Ser
  VAR_SEQ 298: note = X is Cys, Gly, or Ser
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKELKTPLGDTTHTXPRXPAPELLGGPDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 512 aa, synthetic construct
  source 1..512: note = DNAse1 Construct
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 512 aa, synthetic construct
  source 1..512: note = DNAse1 Construct
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSNQTAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 512 aa, synthetic construct
  source 1..512: note = DNAse1 Construct
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPNSTLPFNFQAAYGLSDQLAQAISDHYPVEVMLKGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 534 aa, synthetic construct
  source 1..534: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 534 aa, synthetic construct
  source 1..534: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 555 aa, synthetic construct
  source 1..555: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 21 - protein, 576 aa, synthetic construct
  source 1..576: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 22 - protein, 555 aa, synthetic construct
  source 1..555: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSRAFTNNRKSVSLKKRKKGNRSGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 542 aa, synthetic construct
  source 1..542: note = DNAse1L3 Construct
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGGGGSGGGGSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 557 aa, synthetic construct
  source 1..557: note = DNAse1L3 Construct
  VAR_SEQ 318: note = X is Cys, Gly, or Ser
  VAR_SEQ 321: note = X is Cys, Gly, or Ser
  VAR_SEQ 336: note = X is Cys, Gly, or Ser
  VAR_SEQ 339: note = X is Cys, Gly, or Ser
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSELKTPLGDTTHTXPRXPAPEFLGGPDKTHTXPPXPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 519 aa, synthetic construct
  source 1..519: note = DNAse1L3 Construct
  VAR_SEQ 298: note = X is C, G, or S
  VAR_SEQ 301: note = X is C, G, or S
MSRELAPLLLLLLSIHSALAMRICSFNVRSFGESKQEDKNAMDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSGSDKTHTXPPXPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 535 aa, synthetic construct
  source 1..535: note = DNAse1L3 Construct
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 535 aa, synthetic construct
  source 1..535: note = DNAse1L3 Construct
MSRELAPLLLLLLSIHSALAMRICSFNVRSFGRSKQEDKNATDVIVKVIKRCDIILVMEIKDSNNRICPILMEKLNRNSRRGITYNYVISSRLGRKTYKEQYAFLYKEKLVSVKRSYHYHDYQDGDNDTFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 535 aa, synthetic construct
  source 1..535: note = DNAse1L3 Construct
MSRELAPLLLLLLSIHSALAMRICSFNVRSFGRSKQEDKNATDVIVKVIKRCDIILVMEIKDSNNRICPILMEKLNRNSRRGITYNYTISSRLGRKTYKEQYAFLYKEKLVSVKRSYHYHDYQDGDADVFSREPFVVWFQSPHTAVKDFVIIPLHTTPETSVKEIDELVEVYTDVKHRWKAENFIFMGDFNAGCSYVPKKAWKNIRLRTDPRFVWLIGDQEDTTVKKSTNCAYDRIVLRGQEIVSSVVPKSNSVFDFQKAYKLTEEEALDVSDHFPVEFKLQSSRAFTNSKKSVTLRKKTKSKRSGSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 284 aa, synthetic construct
  source 1..284: note = Mouse DNAse1
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKI

SEQ ID NO: 30 - protein, 310 aa, synthetic construct
  source 1..310: note = Mouse DNAse1L3
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRS

SEQ ID NO: 31 - protein, 222 aa, synthetic construct
  source 1..222: note = hIgG1 Fc domain, Fc mouse
GCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 32 - protein, 513 aa, synthetic construct
  source 1..513: note = Sequence 1171, mouse DNAse1 construct
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 33 - protein, 513 aa, synthetic construct
  source 1..513: note = Sequence 1671, mouse DNAse1 construct
MRYTGLMGTLLTLVNLLQLAGTLRIAAFNIRTFGETKMSNATLSVYFVKILSRYDIAVIQEVRDSHLVAVGKLLDELNRDKPDTYRYVVSEPLGRKSYKEQYLFVYRPDQVSILDSYQYDDGCEPCGNDTFSREPFIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 34 - protein, 513 aa, synthetic construct
  source 1..513: note = Sequence 1687, mouse DNAse1 construct
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYNLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 35 - protein, 513 aa, synthetic construct
  source 1..513: note = Sequence 1689, mouse DNAse1 construct
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQTAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 36 - protein, 539 aa, synthetic construct
  source 1..539: note = Sequence 1584, mouse DNAse1L3 construct
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 37 - protein, 519 aa, synthetic construct
  source 1..519: note = Sequence 1596, mouse DNAse1L3 construct
MSLHPASPRLASLLLFILALHDTLALRLCSFNVRSFGASKKENHEAMDIIVKIIKRCDLILLMEIKDSSNNICPMLMEKLNGNSRRSTTYNYVISSRLGRNTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 38 - protein, 539 aa, synthetic construct
  source 1..539: note = Sequence 1615, mouse DNAse1L3 construct
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSSGVFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 39 - protein, 539 aa, synthetic construct
  source 1..539: note = Sequence 1669, mouse DNAse1L3 construct
MSLHPASPRLASLLLFILALHDTLALRLCSFNVRSFGRSKKENHEAMDIIVKIIKRCDLILLMEIKDSSNNICPMLMEKLNGNSRRSTTYNYVISSRLGRKTYKEQYAFVYKEKLVSVKTKYHYHDYQDGDTDVFSREPFVVWFHSPFTAVKDFVIVPLHTTPETSVKEIDELVDVYTDVRSQWKTENFIFMGDFNAGCSYVPKKAWQNIRLRTDPKFVWLIGDQEDTTVKKSTSCAYDRIVLCGQEIVNSVVPRSNGTFDFQKAYDLSEEEALDVSDHFPVEFKLQSSRAFTNNRKSVSLKKRKKGNRSSSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 40 - protein, 7 aa, synthetic construct
  source 1..7: note = Linker
SSTMVRS

SEQ ID NO: 41 - protein, 7 aa, synthetic construct
  source 1..7: note = Linker
SSTMVGS

SEQ ID NO: 42 - protein, 284 aa, synthetic construct; identical to SEQ ID NO: 29
  source 1..284: note = Mouse DNAse1

SEQ ID NO: 43 - protein, 310 aa, synthetic construct; identical to SEQ ID NO: 30

SEQ ID NO: 44 - protein, 512 aa, synthetic construct
  source 1..512: note = Sequence 1833, human DNAse1 construct
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSDQLAQAISDHYPVEVMLKGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 228 aa, synthetic construct
  source 1..228: note = hIgG1 Fc domain, Fc human
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 512 aa, synthetic construct
  source 1..512: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 512 aa, synthetic construct
  source 1..512: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 512 aa, synthetic construct
  source 1..512: note = Human DNAse1 Construct
MRGMKLLGALLALAALLQGAVSLKIAAFNIQTFGRTKMSNATLVSYIVQILSRYDIALVQEVRDSHLTAVGKLLDNLNQDAPDTYHYNVSEPLGRNSYKERYLFVYRPNQTSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYNLSNQTAQAISDHYPVEVMLKGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 554 aa, synthetic construct
  source 1..554: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 50 - protein, 533 aa, synthetic construct
  source 1..533: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKRAFTNNRKSVSLKKRKKGNRS

SEQ ID NO: 51 - protein, 533 aa, synthetic construct
  source 1..533: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKRAFTNNRKSVSLKKRKKGNRSGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 520 aa, synthetic construct
  source 1..520: note = Human DNAse1 Construct
MRGMKLLGALLALAALLQGAVSLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLKGGGGSGGGGSSDKTHTSPPSPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 513 aa, synthetic construct
  source 1..513: note = Human DNAse1 Construct
MRGMKLLGALLALAALLQGAVSLKIAAFNIRTFGETKMSNATLVSYIVQILSRYDIALVQEVRDSHLTAVGKLLDNLNQDAPDTYHYVVSEPLGRKSYKERYLFVYRPDQVSAVDSYYYDDGCEPCGNDTFNREPFIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLQGAVVPDSALPFNFQAAYGLSDQLAQAISDHYPVEVMLKGSCSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 37 aa, synthetic construct
  source 1..37: note = X1 peptide
RAFTNNRKSVSLKKRKKGNRSGGVKKPHRYRPGTVAL

SEQ ID NO: 55 - protein, 40 aa, synthetic construct
  source 1..40: note = X1 peptide
KQTARKAPRKQLATKAARKSAPATGGVKKPHRYRPGTVAL

SEQ ID NO: 56 - protein, 7 aa, synthetic construct
  source 1..7: note = Linker
SSTMVHS

SEQ ID NO: 57 - protein, 22 aa, synthetic construct
  source 1..22: note = Signal Sequence
MRYTGLMGTLLTLVNLLQLAGT

SEQ ID NO: 58 - protein, 25 aa, synthetic construct
  source 1..25: note = Signal Sequence
MSLHPASPRLASLLLFILALHDTLA

SEQ ID NO: 59 - protein, 22 aa, synthetic construct
  source 1..22: note = Signal Sequence
MRGMKLLGALLALAALLQGAVS

SEQ ID NO: 60 - protein, 20 aa, synthetic construct
  source 1..20: note = Signal Sequence
MSRELAPLLLLLLSIHSALA

SEQ ID NO: 61 - protein, 19 aa, synthetic construct
  source 1..19: note = Signal Sequence
MTRLTVLALLAGLLASSRA

SEQ ID NO: 62 - DNA, 28 bp, synthetic construct
  source 1..28: mol_type = other DNA
cctctagtgagtggaacacaacttctgc

SEQ ID NO: 63 - DNA, 22 bp, synthetic construct
  source 1..22: mol_type = other DNA
tgcaggcaagctctcttcttgc

SEQ ID NO: 64 - DNA, 20 bp, synthetic construct
  source 1..20: mol_type = other DNA
cgagatcaaactgcaaggcg

SEQ ID NO: 65 - DNA, 20 bp, synthetic construct
  source 1..20: mol_type = other DNA
ccggccgctttgtttaccta

SEQ ID NO: 66 - DNA, 21 bp, synthetic construct
  source 1..21: mol_type = other DNA
ctaaatagcccacacgttccc

SEQ ID NO: 67 - DNA, 20 bp, synthetic construct
  source 1..20: mol_type = other DNA
agagctcccgtgagtggtta

SEQ ID NO: 68 - DNA, 20 bp, synthetic construct
  source 1..20: mol_type = other DNA
tccagagcaggctcaagtgg

SEQ ID NO: 69 - DNA, 20 bp, synthetic construct
  source 1..20: mol_type = other DNA
acctgacacaaataccttgg

SEQ ID NO: 70 - DNA, 20 bp, synthetic construct
  source 1..20: mol_type = other DNA
cgagatcaaactgcaaggcg

SEQ ID NO: 71 - DNA, 20 bp, synthetic construct
  source 1..20: mol_type = other DNA
ccggccgctttgtttaccta

SEQ ID NO: 72 - DNA, 20 bp, synthetic construct
  source 1..20: mol_type = other DNA
ctgttccccaaccttttcct

SEQ ID NO: 73 - DNA, 20 bp, synthetic construct
  source 1..20: mol_type = other DNA
ccatgattgtgaggggtagg

SEQ ID NO: 74 - protein, 513 aa, synthetic construct
  source 1..513: note = Construct 1687b mouse DNAse1 Construct
MRYTGLMGTLLTLVNLLQLAGTLRIAAFNIRTFGRTKMSNATLSVYFVKILSRYDIAVIQEVRDSHLVAVGKLLDELNRDKPDTYRYNVSEPLGRKSYKEQYLFVYRPDQVSILDSYQYDDGCEPCGNDTFSREPAIVKFFSPYTEVQEFAIVPLHAAPTEAVSEIDALYDVYLDVWQKWGLEDIMFMGDFNAGCSYVTSSQWSSIRLRTSPIFQWLIPDSADTTVTSTHCAYDRIVVAGALLQAAVVPNSAVPFDFQAEYGLSNQLAEAISDHYPVEVTLRKISSTMVGSGCKPCICTVPEVSSVFIFPPKPKDVLYITLEPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

(19) United States
(12) Patent Application Publication
(10) Pub. No.: US 2026/0083828 A1
(43) Pub. Date: Mar. 26, 2026

(54) COMPOSITIONS AND METHODS FOR TREATING, AMELIORATING, AND/OR PREVENTING DISEASES AND/OR DISORDERS

(71) Applicant: Yale University, New Haven, CT (US)

(72) Inventors: Demetrios Braddock, New Haven, CT (US); Paul Stabach, New Haven, CT (US); Ethan Lester, New Haven, CT (US)

(21) Appl. No.: 19/326,001

(22) Filed: Sep. 11, 2025

Related U.S. Application Data
(63) Continuation of application No. PCT/US2024/019435, filed on Mar. 11, 2024.
(60) Provisional application No. 63/489,749, filed on Mar. 11, 2023.
(60) Provisional application No. 63/489,750, filed on Mar. 11, 2023.
(60) Provisional application No. 63/489,751, filed on Mar. 11, 2023.
(60) Provisional application No. 63/591,832, filed on Oct. 20, 2023.

(51) Int. Cl. A61K 38/46; C12N 9/22

(52) U.S. Cl. CPC A61K 38/465; C12N 9/22; C12Y 301/21001; C07K 2319/30

(57) ABSTRACT

The present disclosure includes compositions and methods for treating, ameliorating, and/or preventing certain diseases and/or disorders associated with associated with DNAse1 and/or DNAse1L3 deficiency.

### CROSS-REFERENCE TO RELATED APPLICATIONS

This application is a continuation of, and claims priority to, International Application No. PCT/US2024/019435, filed Mar. 11, 2024, which claims priority under 35 U.S.C. § 119(e) to U.S. Provisional Patent Application No. 63/489,749, filed Mar. 11, 2023; U.S. Provisional Patent Application No. 63/489,750, filed Mar. 11, 2023; U.S. Provisional Patent Application No. 63/489,751, filed Mar. 11, 2023; and U.S. Provisional Patent Application No. 63/591,832, filed Oct. 20, 2023, each of which is hereby incorporated by reference in its entirety.

### SEQUENCE LISTING

This disclosure contains one or more sequences in a computer readable format in an accompanying file titled “047162-7446WO1_Sequence_Listing.xml”, which is 100,200 Bytes in size and was created Mar. 11, 2024, the contents of which are incorporated herein by reference in their entireties.

### BACKGROUND

Lupus erythematosus (or lupus) is a general term for a group of autoimmune diseases in which the human immune system becomes hyperactive and attacks healthy tissues. Symptoms of these diseases can affect many different body systems, including joints, skin, kidneys, blood cells, heart, and lungs. The most common and most severe form of lupus is systemic lupus erythematosus (SLE).

Lupus symptoms vary from person to person and may occur sporadically. Common symptoms are joint pain and swelling, and arthritis, mostly in the fingers, hands, wrists, and knees. Other common symptoms include chest pain during respiration; oral ulcer; fatigue; weight loss; fever with no other cause; general discomfort, uneasiness, or ill feeling (malaise); hair loss; sensitivity to sunlight; a “butterfly” facial rash, seen in about half people with SLE; and swollen lymph nodes. Lupus is a high morbidity condition, with most patients experiencing life-threatening nephritis.

Lupus is thought to be influenced by multiple genes and gene polymorphisms, more than 30 of which have now been linked with the disorder. SLE pathogenesis is related to a reduced ability to clear DNA released from apoptotic cells, the accumulation of which over time triggers an autoimmune response and the formation of the anti-DNA autoantibodies. There are two key features associated with SLE: dysregulated activation of both T and B lymphocytes and the development of anti-DNA autoantibodies—particularly against double-stranded DNA—that are involved in tissue damage. Major histocompatibility complex (MHC) class II genes, certain class III genes [complement components 2 (C2) and 4 (C4A), tumor necrosis factor (TNF) and heat shock protein 70 kD (HSPA1A) alleles] and other non-MHC genes [receptors for Fc fragment of IgG, low affinity IIa and III (FCGR2A and FCGR3A), interleukins 6 and 10 (IL6 and IL10) and B-cell CLL/lymphoma 2 (BCL2)] can each contribute to susceptibility. However, the principal loci and alleles responsible for disease susceptibility are unknown at this time.

Recently a rare autosomal recessive form of SLE with a null mutation in the DNAse1L3 gene was described (Al-Mayouf, et al., 2011, Nature Genetics 43(12), 1186-1188). The DNAse1L3-related SLE was pediatric in onset and correlated with a high frequency of lupus nephritis. Indeed, DNAse1L3-deficient mice were found to rapidly develop antibodies to double-stranded DNA and chromatin, followed later by immune activation, IgG deposition in the kidney glomeruli, and glomerulonephritis. DNAse1L3 is one of three human homologs of DNase I; this enzyme functions as an endonuclease capable of cleaving both single- and double-stranded DNA, is not inhibited by actin, and mediates the breakdown of DNA during apoptosis. On the other hand, DNAse1 binds actin monomers with very high (sub-nanomolar) affinity and actin polymers with lower affinity; actin-bound DNase I is enzymatically inactive. DNAse1 cleaves non-complexed DNA, while DNAse1L3 cleaves chromatin.

Unfortunately, stable bioavailable DNAse1 and/or DNAse1L3 enzyme biologics designed to treat lupus associated with DNASE1 and/or DNAse1L3 deficiency, as well as other pathologies associated with DNAse1 and/or DNAse1L3 deficiency have not been described to date in the literature.

Coronavirus disease 2019 (COVID-19) is an infectious disease caused by a recently isolated virus known as severe acute respiratory syndrome coronavirus 2 (SARS-CoV-2). COVID-19 is an ongoing global pandemic, which has sickened about 4.5 million people and killed more than 300,000 people worldwide. Currently there are no available vaccines or antiviral treatments for the treatment or prevention of COVID-19.

Common symptoms of COVID-19 include fever, cough, fatigue, shortness of breath, and loss of smell and taste. Most COVID-19 infections result in mild symptoms and resolve on their own, but some cases progress to acute respiratory distress syndrome (ARDS), pneumonia, multi-organ failure, systemic inflammation, septic shock, heart failure, arrhythmias, and blood clots, and eventually death.

Acute kidney injury (AKI) is a sudden episode of kidney failure or kidney damage that can happen over the period of a few hours or a few days. AKI causes a build-up of waste products in blood and makes it hard for the kidneys to keep the right balance of fluid in the body. AKI can also affect other organs such as the brain, heart, and lungs. Generally, it occurs due to damage to the kidney tissue caused by decreased kidney blood flow (kidney ischemia) from any cause (e.g., low blood pressure), exposure to substances harmful to the kidney, an inflammatory process in the kidney, and/or an obstruction of the urinary tract that impedes the flow of urine.

AKI may lead to certain complications, including metabolic acidosis, high potassium levels, uremia, changes in body fluid balance, and effects on other organ systems, including death. AKI complicates recovery from cardiac surgery in up to 30% of patients. AKI injures and impairs the function of the brain, lungs, and gut, and places patients at a 5-fold increased risk of death during hospitalization. AKI requiring renal replacement therapy occurs in 2-5% of patients following cardiac surgery and is associated with 50% mortality. For those who recover from renal replacement therapy or even mild AKI, progression to chronic kidney disease in the ensuing months and years is more likely than for those who do not develop AKI. AKI also has significant independent associations with length of stay, costs of hospitalization, and mortality, and the duration and severity of AKI predict higher risk of long-term mortality.

Data from patients and animal models indicate that AKI adversely affects the lungs. Respiratory complications are common in patients with AKI and include pulmonary edema (such as, but not limited to, cardiogenic {hydrostatic, from fluid overload and/or cardiac dysfunction} and/or non-cardiogenic {e.g., endothelial injury from inflammation; and/or endothelial cell death from apoptosis}), impaired lung fluid clearance, and/or respiratory failure requiring mechanical ventilation (such as, but not limited to, prolonged duration of mechanical ventilation and/or prolonged weaning from mechanical ventilation). The development of respiratory failure in patients with AKI greatly increases the risk of death.

The syndrome of diffuse alveolar hemorrhage (DAH) is characterized by bleeding into the alveolar spaces of the lungs. DAH is caused at least in part by disruption of the alveolar-capillary basement membrane. This disruption is caused by injury or inflammation of the arterioles, venules, or alveolar septal (alveolar wall or interstitial) capillaries. Hemoptysis (expectoration of blood originating from the lower respiratory tract, i.e., from below the vocal cords) is the usual presenting symptom of DAH. However, such expectoration may be absent at presentation in one-third of patients, even when hemorrhage is severe.

The onset of DAH is most often abrupt or of short duration (less than seven days). Common initial symptoms are often nonspecific and may include cough, hemoptysis, fever, and dyspnea. Some patients develop acute severe respiratory distress syndrome (ARDS), requiring immediate ventilatory support with mechanical ventilation. In fact, DAH is generally considered to be the underlying condition of ARDS. DAH is a severe disease with a 50-75% mortality rate.

Unfortunately, there is no specific treatment for DAH. When an underlying cause is identified, specific treatment of that underlying disease or process is appropriate as available but may not resolve DAH. Inflammatory causes of DAH (such as rheumatic diseases, differentiation [retinoic acid] syndrome, and some drug-induced) are often treated with systemic glucocorticoids, with additional immunosuppressive therapy offered in severe cases. Patients with DAH typically have hypoxemia (lower than normal oxygen levels in blood) requiring supplemental oxygen. Hypoxemia can be severe enough to require noninvasive or invasive mechanical ventilation, including extra-corporeal membrane oxygenation (ECMO). ECMO's use in this context is controversial due to the necessity for anticoagulation and the attendant risk of worsening alveolar hemorrhage.

Aicardi-Goutieres syndrome (AGS) was originally defined as an early onset, progressive encephalopathy characterized by intracranial calcification and microcephaly, white matter disease, and cerebrospinal fluid lymphocytosis—these presentations were suggestive of an inflammatory process. Over time, other features were recognized as consistent associations, most frequently chilblain-like skin lesions, glaucoma, hypothyroidism, and lupus-like disease.

Mutations in the TREX1 (AGS1), RNASEH2A (AGS4), RNASEH2B (AGS2), and RNASEH2C (AGS3) genes may cause AGS. Significant clinical differences may exist between patients, both within families and across genotypes. As a result, none of the core signs as originally delineated are now considered necessary for the diagnosis.

The AGS-associated genes encode proteins involved in metabolism and/or sensing of nucleic acids (DNA and RNA), and mutations in these genes result in the induction of the antiviral cytokine type I interferon, which has been hypothesized to be directly relevant to pathogenesis. The term “type I interferonopathy” is often used to describe the wider spectrum of disease that results from dysfunction of these genes, implying the possibility of a common “anti-interferon” approach to therapy.

Most affected individuals experience significant intellectual and physical problems, although this is not always the case. The clinical features of AGS can mimic those of in utero acquired infection, and some characteristics of the condition also overlap with the autoimmune disease systemic lupus erythematosus (SLE). The initial description of AGS suggested that the disease was always severe, and was associated with unremitting neurological decline, resulting in death in childhood. As more cases have been identified, it has become apparent that this is not necessarily the case, with many patients now considered to demonstrate an apparently stable clinical picture, alive in their 4^{th} decade. Moreover, rare individuals with pathogenic mutations in the AGS-related genes can be minimally affected (perhaps only with chilblains) and are in mainstream education, and even affected siblings within a family can show marked differences in severity.

AGS may present as the following distinct syndromes: (1) “classic” AGS with prenatal or infantile onset, representing a remarkable mimic of trans-placentally acquired infection (“pseudo-TORCH”) with irritability, severe intractable pain, feeding difficulties, microcephaly, abnormal movements, and epileptic seizures, as well as hematological disturbances such as thrombocytopenia, anemia, and liver dysfunction, associated with white matter disease, brain mass loss, microcephaly, and intracranial calcification on neuroimaging; (2) Disease presenting beyond the first year of life, with either the subacute onset of profound neurological regression (otherwise similar to classic AGS) or with a more slowly progressive, variable combination of spasticity and dystonia associated with normal neuroimaging or the presence of non-specific white matter changes and/or intracranial calcification; (3) Dystonia and neuroimaging features characteristic of bilateral striatal necrosis, manifest at a few months of age or in later childhood, almost exclusively due to ADAR1 mutations (in certain embodiments, ADAR1-related disease can be considered in any child presenting with bilateral striatal necrosis or otherwise unexplained subacute onset dystonia); (4) Patients with slowly progressive (‘non-syndromic’) spastic paraparesis confined to the lower limbs due to mutations in ADAR1, IFIH1, and RNASEH2B, some with completely normal spinal and cranial imaging; and (5) Intracerebral, large vessel disease including Moyamoya disease and aneurysms with intracerebral haemorrhage and infarcts, representing a particular feature of SAMHD1-related disease. Some affected individuals have completely normal psychomotor development, perhaps only demonstrating chilblain like lesions of the skin before presentation of a cerebrovasculopathy. The pathogenesis of these syndromes is believed to be due to an alpha-interferonopathy driven by undampened or increased signaling through the STING pathway.

There is thus a need in the art to identify therapeutic agents and treatments that can be used to treat, ameliorate, and/or prevent diseases and/or disorders contemplated herein. The present disclosure addresses and meets this need.

There is thus a need in the art to identify therapeutic agents and treatments that can be used to treat, ameliorate, and/or prevent DAH in an afflicted and/or susceptible subject. The present disclosure addresses and meets this need.

### BRIEF SUMMARY

The disclosure provides a method of treating, ameliorating, and/or preventing DAH in a subject.

In certain embodiments, the method comprises administering to the subject a therapeutically effective amount of a construct comprising the amino acid sequence:

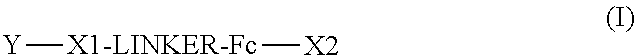

wherein Y, X1, LINKER, X2, and Fc are defined elsewhere herein.

Furthermore, the present disclosure provides certain constructs comprising DNAse1 and/or DNAse1L3 as defined elsewhere herein.

The present disclosure provides methods of treating, ameliorating, and/or preventing forms of lupus associated with DNAse1 and/or DNAse1L3 deficiency in a subject using certain constructs of the disclosure. Further, the present disclosure provides methods of treating, ameliorating, and/or preventing diseases and/or disorders associated with inefficient NET hydrolysis (NETolysis) in a subject using certain constructs of the disclosure. Further, the present disclosure provides methods of treating, ameliorating, and/or preventing an autoimmune disorder associated with DNAse1 and/or DNAse1L3 deficiency in a subject using certain constructs of the disclosure. Further, the present disclosure provides methods of treating, ameliorating, and/or preventing pathologic thrombosis in a subject using certain constructs of the disclosure. Further, the present disclosure provides methods of treating, ameliorating, and/or preventing a myocardial infarction in a subject using certain constructs of the disclosure. Further, the present disclosure provides methods of treating, ameliorating, and/or preventing cancer metastasis in a subject using certain constructs of the disclosure.

The disclosure provides a method of treating, ameliorating, and/or preventing inefficient NET hydrolysis (“NETolysis” or “NETosis”) in a subject afflicted with a bacterial and/or viral infection.

The disclosure provides a method of treating, ameliorating, and/or preventing systemic inflammation, organ damage, and/or sepsis in a subject afflicted with a bacterial and/or viral infection.

The disclosure provides a method of treating, ameliorating, and/or preventing pathologic thrombosis in a subject afflicted with a bacterial and/or viral infection.

The disclosure provides a method of treating, ameliorating, and/or preventing lung injury, such as but not limited to acute lung injury (ALI), in a subject afflicted with acute kidney injury (AKI). In certain embodiments, the ALI comprises at least one of non-cardiogenic pulmonary edema, impaired lung fluid clearance, and respiratory failure requiring mechanical ventilation.

The disclosure provides a method of reducing, reversing increase of, and/or suppressing levels of IL-6 in a subject.

The disclosure provides a method of reducing, reversing increase of, and/or suppressing levels of IL-6 in a subject afflicted with acute kidney injury (AKI).

The disclosure provides a method of treating, ameliorating, and/or preventing DAH in a subject.

The disclosure provides a method of treating, ameliorating, and/or preventing AGS in a subject.

In certain embodiments, the method comprises administering to the subject a therapeutically effective amount of a construct of the disclosure.

### BRIEF DESCRIPTION OF THE DRAWINGS

The following detailed description of illustrative embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1 illustrates a non-limiting DNAse1-Fc construct of the disclosure, with certain contemplated point mutations highlighted.

FIG. 2 illustrates a non-limiting DNAse1L3-Fc construct of the disclosure, with certain contemplated point mutations highlighted.

FIG. 3 illustrates a non-limiting DNAse1-Fc construct of the disclosure, with certain contemplated point mutations highlighted.

FIG. 4 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life. The non-limiting alignment of amino acid sequences of mouse DNAse1 (SEQ ID NO:42) and mouse DNAse1L3 (SEQ ID NO:43) is illustrated.

FIG. 5 illustrates non-limiting constructs of the disclosure, with certain contemplated point mutations highlighted. In certain embodiments, the construct lacks at least a portion of the DNAse1L3 nuclear localization domain.

FIG. 6 illustrates a non-limiting construct of the disclosure. In certain embodiments, the DNAse1 polypeptide is fused with the C-terminus tail of DNAse1L3.

FIG. 7 illustrates a non-limiting enzyme optimization pathway to be applied to certain constructs of the disclosure, as illustrated with an exemplary protein and/or polypeptide.

FIG. 8 illustrates a non-limiting DNAse1-Fc construct of the disclosure, with certain contemplated point mutations highlighted.

FIG. 9A illustrates microscopic images of lung tissues for normal lungs and DAH lungs.

FIG. 9B illustrates % oxygen measurements for WT mice, DNAse1 and DNAse1L3 double knock-out mice (DKO) afflicted with DAH, and DKO mice afflicted with DAH and treated with a construct of the disclosure. FIG. 9C illustrates lung gross histology of normal lungs and DAH lungs. FIG. 9D illustrates a survival study for DKO and WT mice afflicted with DAH (induced by pristane administration at day 0) with one cohort of DKO mice treated with a construct of the disclosure (top line) at day 0. The results demonstrate the increased susceptibility of DKO mice to DAH mortality (bottom line) compared to WT mice (middle line), and the increased survival of DKO mice treated with the construct of the disclosure (top line).

FIG. 10 illustrates a survival study for WT C57BL/6J mice (C57BL6 mice obtained from Jackson Labs) afflicted with DAH (induced by pristane administration at day 0) and treated with a construct of the disclosure starting at day 10, after first animal died (and then at day 17 and day 24). The construct-treated mice showed statistically significant higher survival rates than the untreated mice. The results demonstrate that WT mice, and by extension humans without monogenic disorders in DNAse genes, can potentially be rescued from DAH-induced mortality by a DNA degrading biological agent, such as the constructs of the disclosure, during pathologies associated with aberrant NETosis pathologies.

FIG. 11 illustrates a survival study for WT C57BL/6Tac mice (C57BL6 mice obtained from Taconic Labs) afflicted with DAH (induced by pristane administration at day 0) and treated with a construct of the disclosure starting at day 9 (after the first animal dies), and then day 17 and day 24. The construct-treated mice showed statistically significant higher survival rates than the untreated mice, again supporting the notion that the construct of the invention can rescue from individuals from DAH-induced mortality.

FIGS. 12A-12B illustrate measurements of D-dimer (which is a fibrin degradation product) at day 12 for DAH-induced mice (circles—pristane). Coagulopathies such as disseminated intravascular coagulopathy are know to induce morbidity and mortality in DAH patients. Elevated D-dimers are predictive biomarkers for disseminated intravascular coagulation (DIC) and COVID-19 coagulopathies. The results indicate that DIC is present in the pristane-induced murine model of DAH and correlates directly with weight loss and death, simulating the coagulopathies experienced in humans suffering from DAH.

FIG. 13 illustrates a survival study for WT C57BL/6NTac mice afflicted with DAH (induced by pristane administration at day 0) and treated with a construct of the disclosure starting at day 9 (and then at day 14 and day 21). Weights of the mice were measured on days 5, 10, 12, 18, 21, and 24. The results indicate that both treated and untreated DAH mice suffer significant weight loss, but significantly more mice treated with the subject of the invention rescues are rescued from death (FIG. 11). This demonstrates that the constructs of the invention provide significant benefit even to critically ill animals. Mice that died are are represented by symbols red (dark grey). Mice that survived are represented by symbols in cyan (light grey).

FIG. 14 illustrates measurements of C-reactive protein, calprotectin, and SP-D in WT mice with DAH induced by pristane that are treated with vehicle (PBS) or a construct of the disclosure (Construct 1687). Calprotectin, a neutrophil cytosolic protein, was elevated in both treated and untreated mice demonstrating equivalent NETosis in both groups. However, Surfactant Protein D, a marker of pulmonary permeability, was significantly less in the mice dosed with the construct of the disclosure (Construct 1687). The data demonstrate that equivalent levels of NETosis are present in treated and untreated animals, but lung damage is greater in the untreated animals due greater levels of surfactant protein D in the blood of the untreated animals. This supports the finding that the constructs of the invention can prevent destructive inflammation in DAH, which leads to destruction of the alveolar walls.

FIG. 15 illustrates weight loss for DAH mice treated with a construct of the disclosure in which individual animals which drop significant weight are followed throughout the 31 day experiment. A surviving vehicle-treated mouse is followed with Line A, and a surviving treated mice is followed with Line B. In general, mice dosed with the construct of the invention showed significantly less weight loss at days 12 and 18 as compared to untreated DAH mice. Four mice of each cohort dropped weight but fully recovered by day 31. The data demonstrate that the construct of the invention is able to rescue mortality even in critically ill animals that drop significant body weight, as demonstrated by the 30% weight loss exhibited by the treated animal that survived pristane induce DAH mortality (Line B). In comparison the most severe weight loss exhibited by a surviving vehicle-treated animal was 20% (Line A).

FIG. 16 illustrates waterfall plots illustrating the weight loss in mice after pristane-induced DAH, with mortality labelled in red/dark grey bars). Shown also is selected lung gross histology representing least effected (Best) and most effected (Worst) treated (Construct 1687) and untreated (PBS) mice. The data demonstrate that significantly greater number of vehicle treated animals (labelled ‘PBS’) suffered severe weight loss than treated animals (labelled ‘1687’).

FIG. 17 illustrates a non-limiting DNAse1-Fc construct of the disclosure, with certain contemplated point mutations highlighted.

FIG. 18 illustrates a non-limiting DNAse1L3-Fc construct of the disclosure, with certain contemplated point mutations highlighted.

FIG. 19 illustrates a non-limiting DNAse1-Fc construct of the disclosure, with certain contemplated point mutations highlighted.

FIG. 20 illustrates non-limiting constructs of the disclosure, with certain contemplated point mutations highlighted. In certain embodiments, certain mutations render the rDNAse hyperactive and/or render the rDNAse actin-resistant (i.e., has decreased affinity for actin) and/or increase the construct's half-life. The non-limiting alignment of amino acid sequences of mouse DNAse1 (SEQ ID NO:42) and mouse DNAse1L3 (SEQ ID NO:43) is illustrated.

FIG. 21 illustrates non-limiting constructs of the disclosure, with certain contemplated point mutations highlighted. In certain embodiments, the construct lacks at least a portion of the DNAse1L3 nuclear localization domain.

FIG. 22 illustrates a non-limiting construct of the disclosure. In certain embodiments, the DNAse1 polypeptide is fused with the C-terminus tail of DNAse1L3.

FIG. 23 illustrates a non-limiting enzyme optimization pathway to be applied to certain constructs of the disclosure, as illustrated with an exemplary protein and/or polypeptide.

FIG. 24 illustrates a non-limiting DNAse1-Fc construct of the disclosure, with certain contemplated point mutations highlighted.

FIG. 25 illustrates the finding that Construct 1833 effectively promoted NET degradation in a mouse model (DNAse1 and DNAse1L3 double knock-out mice or DKO). Construct 1833 was administered at the same dose as Construct 1687 and resulted in more complete NET degradation than Construct 1687 (see mouse 1 vs. mouse 3 or 4). Interestingly, mouse 2 had been previously dosed with Construct 1687 and was found to have developed an immune response to Construct 1687; subsequence administration of mouse 2 with Construct 1833 showed much improved NET degradation.

FIG. 26A-FIG. 26C illustrate the pharmacodynamic activity of various purified DNASE1 isoforms evaluated in vivo by dosing DKO mice with a single s.c. injection of each biologic at 1 mg/kg and withdrawing blood from the mice at two (FIG. 26A), six (FIG. 26B), and 11 days (FIG. 26C) following dosing. Plasma was isolated from the blood samples and exogenous free DNA and chromatin was added. The samples were then incubated at 37° C. for five minutes, and run on agarose gels to image degradation of the exogenous DNA (or lack thereof) of each isoform at various time points. Most biologics exhibited full PD activity 2 days after dosing, and three isoforms—1671, 1689, and 1687—exhibited full PD activity 6 days after dosing. Plasma from these three mice was drawn 11 days after dosing, revealing the murine isoforms with the longest PD activity to be 1687 and 1689.

FIG. 27 illustrates the prevention of autoimmunity in DKO mice by 1687. WT mice treated with weekly injections of PBS, and DKO mice treated with weekly s.c. doses of PBS (vehicle) or 1687 at 1 mg/Kg beginning on the second week of life. Plasma samples were taken at 8, 14, 25, and 40 weeks and titers of anti-nuclear, anti-histone, and anti-dsDNA autoantibodies were evaluated with by ELISA. In comparison to WT controls, by 8 weeks of age the PBS treated DKO mice (DKO) spontaneously elevated anti-dsDNA, anti-histone, and anti-nuclear antibodies, whereas 1687 treated DKO mice (1687) did not elevate auto-antibodies suggestive of lupus. At 40 weeks all mice were challenged with pristane (500 ul IP), and animals were followed over the next 12 weeks. At 52 weeks the ANAs in the surviving DKO mice remained elevated, however anti-dsDNA abs were no longer elevated, demonstrating increased mortality in vehicle treated DKO mice with elevated anti-dsDNA antibody titers. *p<0.05, **p<0.01, ***p<0.001, ****p<0.0001, ANOVA Kruskal-Wallis test.

FIG. 28A-FIG. 28D illustrate the plasma biomarkers of DKO mice following pristane challenge. FIG. 28A presents serum anti-MPO antibodies in pristane treated lupus mice 4-weeks after pristane treatment (at 44 weeks). FIG. 28B presents creatinine levels in pristane treated lupus mice 4-weeks after pristane treatment (at 44 weeks). FIG. 28C presents quantitative PCR of free DNA in pristane treated lupus mice 4-weeks after pristane treatment (at 44 weeks). *p<0.05, **p<0.01, ***p<0.001, ****p<0.0001, ANOVA Kruskal-Wallis test (FIG. 28A-FIG. 28C). FIG. 28D presents C3 complement levels in the lupus mice at 52 weeks. *p<0.05 Students two tailed T-test, vehicle (DKO) and 1687 treated (1687) DKO mice.

FIG. 29A-FIG. 29E illustrate membranous glomerulonephropathy and immune complex deposition in glomeruli of vehicle treated DKO mice. FIG. 29A presents light microscopic exam of the kidneys of untreated DKO mice that revealed the presence of membranous glomerulopathy in some untreated DKO mice (Martius Scarlet Blue stains). These findings were not present in the 1687 treated DKO cohort. FIG. 29B presents examination of affected kidneys in the untreated DKO mice that revealed evidence of revealed immunocomplex deposition via immunofluorescence staining with IgG and C1q. FIG. 29C demonstrates that glomerulonephritis assessed in a blinded fashion by a board-certified nephropathologist revealed a lower glomerulonephritis score in 1687 treated DKO mice than in WT controls, but no significant differences in the treated and untreated DKO mice. FIG. 29D demonstrates that spelnomegaly was significantly present in vehicle treated DKO mice in comparison to their 1687 treated siblings, as was erythropoietin (EPO) at 52 weeks. FIG. 29E demonstrates that histologic examination of the spleens revealed white pulp expansion due to coalescence of lymphoid follicles in vehicle treated DKO mice (yellow arrows). Vehicle treated DKO mice also exhibited robust extramedullary hematopoiesis in comparison to 1687 treated siblings and WT controls (cyan arrows). *p<0.05, **p<0.01, ***p<0.001, ****p<0.0001, ANOVA Kruskal-Wallis test.

FIG. 30A-FIG. 30C present survival and plasma biomarkers of mice as follows. FIG. 30A demonstrates that a survival comparison of DNAse1^{-/-} and DNAse1L3^{-/-} mice on C57BL/6J backgrounds following pristane challenge revealed increased mortality in DNAse1L3^{-/-} mice compared to DNAse1−/− counterparts (50% vs less than 10%, respectively, p=0.0046. Mantel-Cox), illustrating the effect of functional loss of DNAse1L3 activity on the acute autoinflammatory phenotype. FIG. 30B demonstrates that the mortality induced by pristane challenge in DKO mice could be prevented with weekly 1 mg/Kg s.c doses of 1687 (100% survival in dosed vs. 25% survival in vehicle treated DKO mice, p=0.039, Mantel-Cox). FIG. 30C demonstrates that the plasma biomarkers in the dosed and undosed DKO mice revealed higher levels of IL6 and erythropoietin (EPO) in the undosed animals, and lower levels of CXCL-9. IL-11 trended higher without significance in this limited (n=4) study. *p<0.05, two tailed Student's unpaired T-test.

FIG. 31 presents a Coomassie stained gel of some of the CHO cell purified proteins used in the present studies. Lanes 1, 2, and 3 show three unsuccessful attempts to purify full-length mouse DNASE1-IgG1-Fc fusion protein (predicted MW of 58 kD) from construct 1171. A cryptic trypsin cleavage site, identified and mutated by a single R290G amino acid substitution, resulted in a well-behaved full-length protein (Lane 4 construct 1585). Lane 5, construct 1671 comprises a hyperactive E35R amino acid substitution. Lanes 6 and 7, show two preparations of construct 1687 with a mobility shift indicative of the successful N-linked glycosylation of the newly engineered glycosylation consensus sites. Similarly, lanes 8 and 9 are two preparations of construct 1689 also glycosylation mobility shift. Lane 10 is human DNASE1-IgG1-Fc, construct 1833.

FIG. 32A-FIG. 32E present data and results related to biochemical characterization of dual activity biologics. FIG. 32A demonstrates that commercial DNASE1 (lane 1, Roche) digests plasmid (top) but not chromatin DNA (bottom), in contrast to 1687 (lane 2) or 1833 (human isoform of 1687, lane 3). Neg—negative control (plasmid DNA only). FIG. 32B demonstrates that 1687 degrades NETs in a concentration dependent manner, in contrast to DNAse1. Quantitation of NET degradation was measured using SYTOX green fluorescent labelling. FIG. 32C demonstrates that 1687 degrades NETs in a concentration dependent manner, in contrast to DNAse1. Quantitation of NET degradation was measured using SYTOX green fluorescent labelling. FIG. 32D demonstrates that 1687 and human analog (1833) are not inhibited by Actin, in contrast to commercial DNASE1 (Roche). FIG. 32E demonstrates the effects of Plasmin, Aprotinin, and Heparin on commercial DNASE1 (lane 1, Roche), 1687 (lane 2), and 1833 (lane 3). a. Histone bound DNA in buffer alone (N/C), and in the presence of DNASE1 (1), 1687 (2), and 1833 (3). Only 1687 and 1883 hydrolyze histone bound DNA. b. Plasmin activates DNASE1 inter-nucleosome chromatin cleavage. c. Plasmin activation of chromatin cleavage by DNAse1 is inhibited by Aprotinin (lane 1), but has no effect on 1687 or 1833 (lanes 2 and 3). b & d. Plasmin and heparin, known inhibitors of DNASE1L3 [49], have no effect on 1687 or 1883 cleavage of Chromatin. The bands present in d are the 18S and 28S Ribosomal subunits which are digested by endogenous RNAse in serum.

FIG. 33 presents chromatographic profiling of Construct 1833. Reverse phase HPLC with a protein BEH C-4 column eluted Construct 1833 as a single peak with a small trailing peak in approximately 46% acetonitrile.

FIG. 34A-FIG. 34B presents characterization of Construct 1833 with size exclusion chromatography—light scattering (SEC-LS). FIG. 34A presents a molar mass distribution plot of Construct 1833 (in red) overlaid with two standards—Bovine Serum Albumin (BSA, MW=66 kDa) and Aldolase (MW=156 kDa). Construct 1833 was run at two concentrations, 0.59 mg/ml and a 20 fold dilution (0.08 mg/ml), revealing a polypeptide mass of 116 kDa and 121 kDa and sugar mass of 14 kDa and 13 kDa, respectively. The analysis yielded a total mass of the biologic at 132 and 134 kDa at the higher and lower concentration, indicated that Construct 1833 exists as a stable dimer in solution. FIG. 34B presents hydrodynamic radius distribution plots of Construct 1833 (red) overlaid with two standards—Aldolase (blue) and Ovalbumin (green). Construct 1833 eluted ahead of Aldolase, confirming a non-spherical/extended shape due to the presence of sugars, yielding a hydrodynamic radius (Rh) of 5.3 nm.

FIG. 35 presents amino acid sequences of a dual acting mouse Dnase1-Fc (Construct 1687) and the human DNASE1-Fc equivalent (Construct 1833). Highlighted features include a signal sequence, a DNAse1 platform sequence, a linker sequence, an IgG1 Fc sequence, mutations introduced into the DNAse backbone to confer DNASE1L3 activity and reduce acting binding, and mutations introduced into the IgG1 Fc domains to enhance FcRN endosomal recycling. In particular with regard to FIG. 35, gold font: Signal sequence; green font: DNASE1 platform sequence; black font: linker sequence; purple font: IgG1 Fc domain; red font: mutations introduced into the DNASE backbone to confer DNASE1L3 activity and reduce acting binding; cyan font: mutations introduced into the IgG1 Fc domains to enhance FcRN endosomal recycling.

FIG. 36 presents images of data related to DKO mouse plasma induction of NETs in WT mouse neutrophils. Neutrophils from WT mice were isolated and plated into poly-lysine-coated wells of a 96-well plate and allowed to adhere at 37° C. in 100 ul RPMI. After 1 hr, the wells were spiked with 10 ul plasma from either a WT (left) or DKO (right) mouse and incubated for 2 hours. After adding Sytox Green (5 uM), images were captured on a fluorescent microscope. The WT neutrophils demonstrated increased fluorescence after being exposed to the plasma from a DKO mouse (right) compared to when the same neutrophils are exposed to the plasma of a WT mouse (left). Image captured at 40× magnification. It was found that plasma from DKO mice induced NETosis in WT murine neutrophils over and above the effects of plasma from WT mice.

FIG. 37 presents images related to immunofluorescence from a lung section of an untreated mouse removed from the study due to severe DAH showing deposition of MPO (green arrows, left panel) and Citrullinated Histone H3 (Cit-H3) (red arrows, middle panel) in the alveolar walls and DAPI stained nuclei in blue. Scale bar=50 uM. Mice which expired from DAH all revealed similar staining.

FIG. 38A-FIG. 38F present data and results related to the efficacy of LBme (Construct 1687) in Diffuse Alveolar Hemorrhage in two strains of C57BL/6 mice. FIG. 38A demonstrates that WT mice had higher amounts of plasma cfDNA 14 days after pristane injection than untreated negative control N/C mice when analyzed by pPCR. ****p<0.0001, Student's two-tailed unpaired T-test. FIG. 38B presents data demonstrating that ten days after pristane injection, before the dosing strategy began, both cohorts of mice showed elevated levels of CXCL10 compared with (N/C) mice that did not receive pristane. FIG. 38C presents data and results related to C57BL/6J mice (The Jackson Laboratory) that were dosed i.p. with 500 ml of pristane on day zero, and weekly with either PBS or LBme (Construct 1687) (1 mg/kg) following the first death of an animal post-pristane challenge (on day 10). The survival rate of dosed and vehicle treated animals was 95% vs 70%, respectively (p=0.029, Mantel-Cox). FIG. 38D presents data and results related to an identical study performed in C57BL/6Tac mice (Taconic Biosciences) with dosing beginning on day 9 post pristane, yielding a survival rate of 70% and 35%, for dosed and vehicle-treated animals respectively (p=0.017, Mantel-Cox). FIG. 38E demonstrates that D-dimers measured in C57BL/6J mice at 14 days inversely correlated with weight gain in the vehicle (slope=−6.35, R²=0.55, F=33.01, p<0.0001) and Lbme-treated (slope=−7.54, R²=0.69, F=63.84, p<0.0001) cohorts. FIG. 38F demonstrates that C-reactive protein and calprotectin were equivalent in the treated and untreated cohorts, but surfactant-D levels were significantly higher in vehicle treated C57BL/6 mice at 14 days after pristane challenge, demonstrating that although acute-phase reaction and NETosis was equivalent in treated and untreated cohorts, LBme (Construct 1687) reduced alveolar damage in the treated mice. *p<0.05, Student's two-tailed unpaired T-test. The results demonstrated that administering Lbme (Construct 1687) to WT mice following the onset of respiratory distress and an initial fatality in the pristane model rescued animals from fatal pulmonary hemorrhage and death due to lung injury and a DIC-related coagulopathy.

FIG. 39A-FIG. 39E present data and results related to the efficacy of 1833 in the blood and plasma of SLE patients. FIG. 39A presents qPCR quantitation of cfDNA in the plasma of 4 SLE patients and 3 healthy controls (HC) incubated with either PBS or 1833. Plasma treated with PBS (blue lines) yielded cycle threshold (Ct) values between 15-25 cycles while plasma treated with 1833 (green lines) yielded Ct values>29 cycles. FIG. 39B presents qPCR analysis of cell free genomic and mitochondrial DNA in whole blood samples from HC and an SLE patient following stimulation with 50 nM PMA that demonstrated significantly increased Ct values in both genomic and mitochondrial cfDNA in the 1833-treated (green) samples compared to the PBS-treated (blue) samples. FIG. 39C presents qPCR analysis of microparticle (MP) associated DNA isolated from an SLE patient that demonstrated increased Ct values in MP-genomic and MP-mitochondrial DNA in the 1833-treated (green) samples compared to the PBS-treated (blue) samples. FIG. 39D presents images of Sytox Green fluorescence of leukocytes isolated from healthy controls and an SLE patient before (untreated) and after PMA stimulation (PMA) that demonstrated increased diffuse fluorescence in SLE leukocytes after PMA treatment, documenting the increased propensity of SLE leukocytes to NET. Following incubation with 1833 the diffuse fluorescence was reduced in the PMA stimulated wells, demonstrating the efficient digestion of NETs by 1833. Scale Bar=100 μM. FIG. 39E presents data related to the reactivity of 1833 in plasma samples of 40 healthy human controls and 99 SLE patients, which demonstrated no significant elevation of anti-1833 antibody levels in SLE vs. HC. *p<0.05, ****p<0.0001, Students two-tailed unpaired T-test.

FIG. 40 presents a graphical representation of data related to digestion of gDNA by 1833 in the Human whole blood of SLE patients. Whole blood with EDTA from an SLE patient and an HC was spiked with 20 mM CaCl2), 20 mM MgCl2 to compensate for the EDTA, and then 200 uM 1833 or PBS was added for a 10 minute incubation at 37° C. The samples were placed on ice and then centrifuged at 1000×g for 10 minutes at 4° C., and plasma was transferred to a new tube from which 1 μl was used in a qPCR reaction, with oligos directed at the human LINE-1 element in quadruplicate as previously described. Untreated samples are shown in blue, and the 1833 treated samples are shown shifted right in green. Some Ct values were not obtained because there was not enough signal to cross the threshold.

FIG. 41A-FIG. 41D present data and results related to digestion of genomic and mitochondrial DNA in human whole blood of SLE patients by Lbme (Construct 1687). Blood was isolated from a lupus patient (LP3) and two healthy controls (HC1+HC2) and placed in 8-well strips with serial dilutions of Lbme (Construct 1687) or an equivalent volume of PBS. After a 10 minute incubation the strips were centrifuged at 1000×g for 5 minutes at 4° C., and 1 ml of plasma was analyzed by qPCR. The data is presented in 4 formats to illustrate the effectiveness of Lbme (Construct 1687) in digesting cfDNA. FIG. 41A presents a representative plot of the Relative Fluorescence Units (RFU) vs the cycle number for Lupus patient #3 (LP3) for both the genomic (left) and mitochondrial (right) profiles. The 8 PBS-treated profiles are shown in green and the 8 titrated Lbme (Construct 1687) profiles are shown in red shifted to the right. The horizontal red lines indicate complete digestion of both genomic and mitochondrial DNA by the higher concentrations of Lbme (Construct 1687). FIG. 41B presents the cycle threshold (Ct) data for each reaction with a conditional formatted heat map to illustrate the relative levels of DNA with blue (low Ct values) predominantly in the PBS-treated samples, indicating an abundance of DNA, and red (high Ct values) predominantly in the Lbme-digested samples, indicating a reduction of DNA. Empty boxes represent a complete absence of detectable DNA. FIG. 41C presents the same data plotted in Prism GraphPad with most Lbme-treated samples absent (undetectable DNA levels that do not cross the Ct), and those which had detectable DNA shown in red. FIG. 41D presents images of representative reactions that were analyzed on a 2% agarose gel stained with ethidium bromide. The genomic amplimer seen without Lbme (Construct 1687) was nearly completely absent in all samples treated with Lbme (Construct 1687), with the exception of LP3 at the lowest Lbme (Construct 1687) concentration. Meanwhile, the mitochondrial amplimer was reduced at most concentrations of Lbme (Construct 1687), but some was still detected at the lower concentrations of Lbme (Construct 1687) tested.

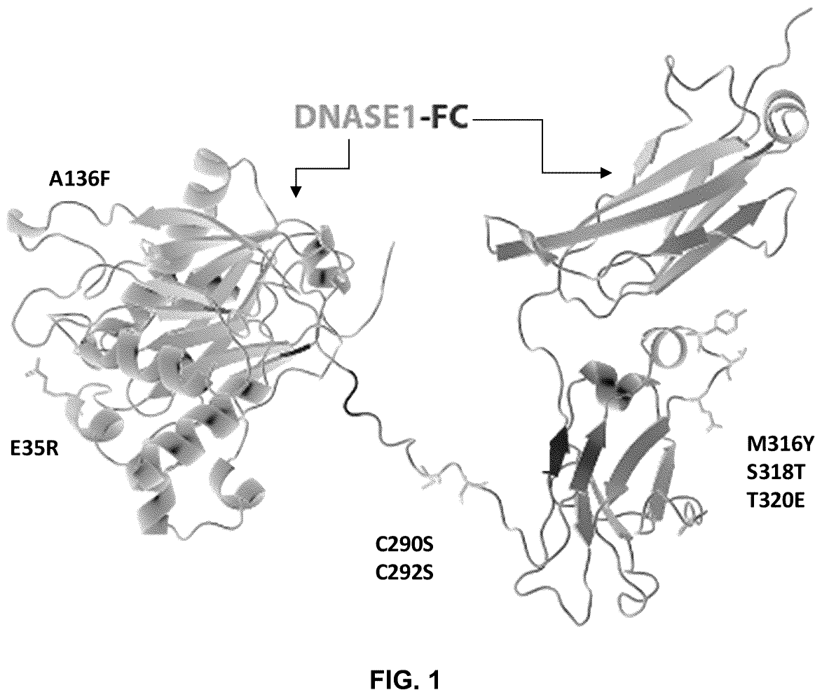

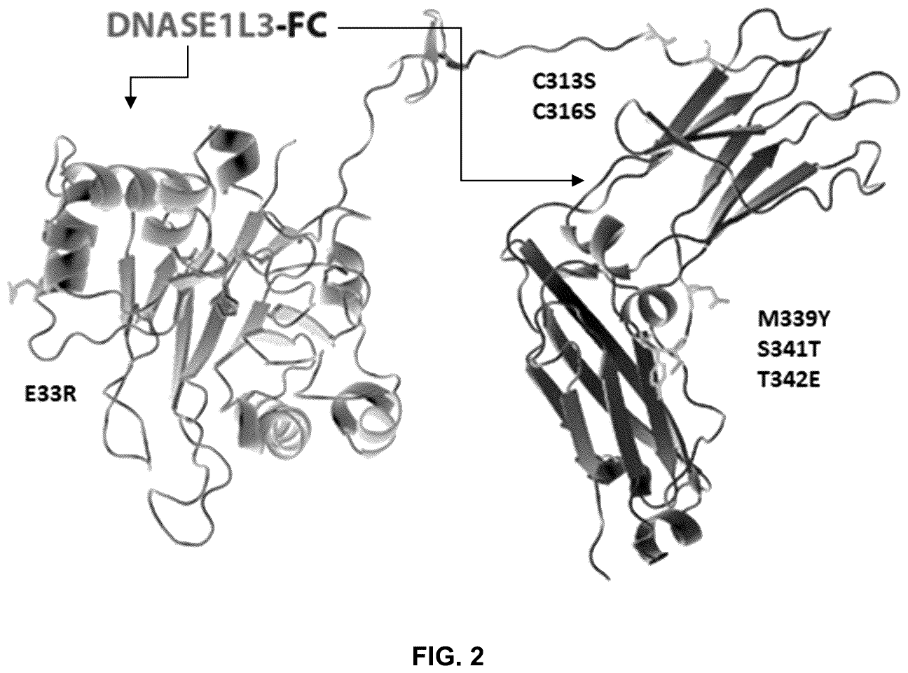

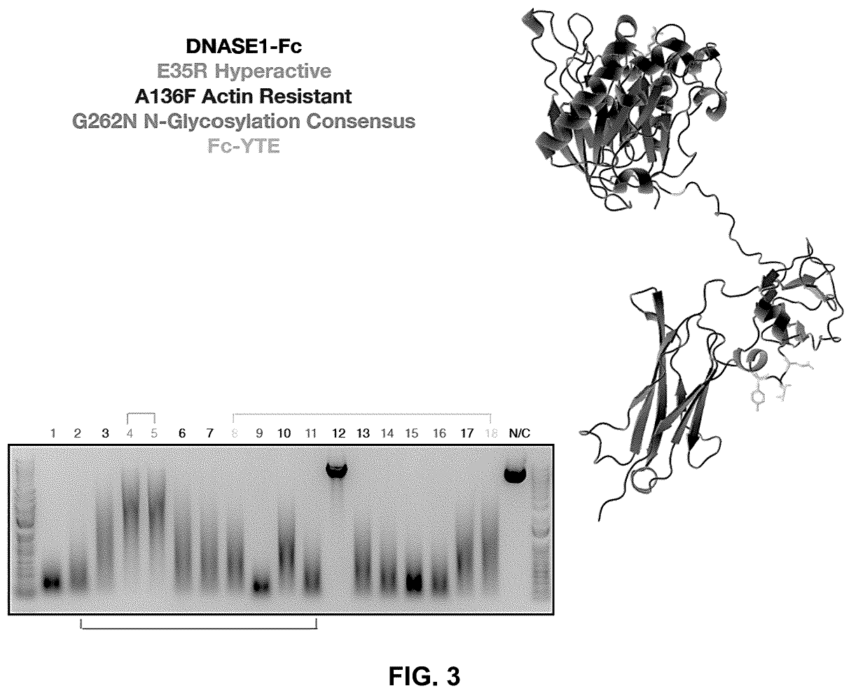

### DETAILED DESCRIPTION OF THE DISCLOSURE

The disclosures of PCT International Application Publication No. WO 2021/142456 published Jul. 15, 2021 (corresponding to PCT/US2021/012990); U.S. Application Publication No. US 2023/0062096 A1 published Mar. 2, 2023 (corresponding to U.S. application Ser. No. 17/792,101); PCT International Application Publication No. WO 2021/252470 published Dec. 16, 2021 (corresponding to PCT/US2021/036368); and U.S. Provisional Patent Application No. 63/512,467 filed Jul. 7, 2023 are included herein in their entireties by reference.

The present disclosure relates, in one aspect, to the discovery that certain constructs can be used to treat, ameliorate, and/or prevent diseases or disorders associated with DNAse1 and/or DNAse1L3 deficiency.

In certain embodiments, the constructs contemplated herein can be used to treat, ameliorate, and/or prevent forms of lupus (including SLE) associated with DNAse1L3 deficiency.

In certain embodiments, the constructs contemplated herein can be used to treat, ameliorate, and/or prevent diseases and/or disorders associated with inefficient NET hydrolysis (“NETolysis”).

In certain embodiments, the constructs contemplated herein can be used to treat, ameliorate, and/or prevent autoimmune disorders such as but not limited to lupus (including SLE), thyroid autoimmune disease, and Hypocomplementeric Urticarial Vasculitis Syndrome (HUVS).

In certain embodiments, the constructs contemplated herein can be used to treat, ameliorate, and/or prevent pathologic thrombosis, such as but not limited to microvascular thrombosis, venous thrombosis, and/or arterial thrombosis. In certain embodiments, the pathologic thrombosis comprises neutrophilic thrombosis, which includes but is not limited to Anti-Neutrophilic Cytoplasmic Autoantibodies (ANCA) vasculitis, Thrombotic thrombocytopenic purpura (TTP), and Bechet's (or Behcet's) disease or syndrome. In certain embodiments, the pathologic thrombosis comprises thrombosis leading to strokes.

In certain embodiments, the constructs contemplated herein can be used to treat, ameliorate, and/or prevent myocardial infarctions.

In certain embodiments, the constructs contemplated herein can be used to treat, ameliorate, and/or prevent spread and progression of cancer (e.g., cancer metastasis).

The present disclosure relates, in one aspect, to the discovery of certain constructs, compositions, and methods for treating, ameliorating, and/or preventing immune mediated pathology associated with a bacterial and/or viral infection.

In certain embodiments, the constructs contemplated herein can be used to treat, ameliorate, and/or prevent inefficient neutrophilic extracellular trap (“NET”) hydrolysis (“NETolysis”) in subject afflicted with a bacterial and/or viral infection.

In certain embodiments, the virus is a coronavirus. In other embodiments, the coronavirus is SARS-Cov and/or SARS-Cov-2.

The present disclosure relates, in one aspect, to the discovery of certain constructs, compositions, and methods for treating, ameliorating, and/or preventing lung injury, such as but not limited to acute lung injury (ALI), in a subject afflicted with acute kidney injury (AKI).

The present disclosure relates, in one aspect, to the discovery of certain constructs, compositions, and methods for treating, ameliorating, and/or preventing Diffuse Alveolar Hemorrhage (DAH).

The present disclosure relates, in one aspect, to the discovery of certain constructs, compositions, and methods for treating, ameliorating, and/or preventing Aicardi-Goutieres syndrome (AGS).

The constructs of the disclosure can be used to treat diseases or disorders that are caused by and/or associated with and/or related to decreased and/or inefficient and/or suboptimal neutrophil extracellular traps (NETs) degradation and/or clearance and/or hydrolysis. Polymorphonuclear leukocytes (PMNs), the most abundant form of white blood cells, circulate in tissues and blood, where they seek out invading micro-organisms. If invading micro-organisms are encountered by PMN's, the cells respond with an array of mechanisms to combat the infection, including phagocytosis, the release of stored antimicrobial compounds in a process called degranulation, and, as a last resort, a self-destructive process wherein the PMN “explodes,” releasing a web of entrapping DNA and cytotoxic material known as “neutrophilic extracellular traps” or NETs. NETs are extracellular, neutrophil-derived DNA webs that trap invading pathogens. The backbone of NETs is a sticky chromatin web attached to which are an assortment of antimicrobial cytotoxic proteins and peptides that are released along with the chromatin when PMN degranulate in response to infectious stimuli. The high concentration of antimicrobial compounds maintained by NETs in close proximity to invading organisms increases the potency of the cytotoxic agents, thereby neutralizing the invading pathogens to prevent their spread and eliminate the threat of infection.

Notwithstanding their beneficial role fighting infection, NETs must be cleared quickly and efficiently from tissues and the circulation, and failure to do so has serious pathologic consequences. Specifically, diseases associated with inefficient NET hydrolysis (“NETolysis”) include autoimmune disorders such as lupus, pathologic thrombosis (such as but not limited to thrombosis leading to strokes), and myocardial infarctions, and the spread and progression of cancer.

NETs are typically degraded by blood-based metalloenzymes, and several circulating enzyme isoforms hydrolyze the high energy bonds in DNA to effect NETolysis. To do so, different enzyme isoforms recognize DNA as free nucleic acid or in associated with proteins such as the chromatin in the protein backbone of NETs. Loss of function mutations in these enzymes have been identified in Systemic Lupus Erythematosus (SLE), including hereditable and highly aggressive forms of SLE presenting in the pediatric population. In addition, NETs foster cancer progression and metastasis, and inhibition of NETs has been shown to decrease cancer metastasis in murine models

Furthermore, the present disclosure relates, in one aspect, to the discovery of certain constructs, compositions, and methods for treating, ameliorating, and/or preventing DAH.

“Neutrophilic extracellular traps” (NETs) comprise entrapped DNA in a sticky chromatin web. Attached to this web are an assortment of proteins and peptides released upon cellular apoptosis in response to infectious stimuli and/or other stimuli (such as but not limited to ischemia). NETs are typically degraded by blood-based metallo-enzymes, and several circulating enzyme isoforms hydrolyze the high energy bonds in DNA to induce “NETosis” or “NETolysis.” To do so, these enzymes recognize DNA as either free nucleic acid or in association with proteins such as the chromatin in the protein backbone of NETs. While NETs play an important role in the immune system, they must also be cleared quickly and efficiently, as failure to do so has serious pathologic consequences. Overwhelming or uncontrolled “NETosis” may lead to systemic inflammation, coagulopathies, and/or remote organ failures.

DAH is triggered in many cases by NETs, which are known to trigger a type-1 interferon response through the STING pathway. This results in cytokine storms leading to alveolar hemorrhage, as has recently been described in some COVID-19 infections. In this disclosure, the accepted animal model of DAH was treated with certain constructs of the disclosure (such as Construct 1687) in both Lupus mice and wild type (WT) mice. The present study showed that the construct of the disclosure at doses of 1 mg/kg per week prevented death from DAH in a murine genetic lupus model.

Moreover, the construct of the disclosure also prevented death from DAH in WT mice in two preventative models: when the treatment was delayed until 10 days after initiation of the disease, and when the first animal in the study had died from DAH. In one aspect, this delayed treatment was designed to model a treatment of the disorder after the initiation of the disease cycle in a therapeutic dosing model.

To validate that the initiation of NETosis had begun before treatment, calprotectin (cytoplasmic protein present in neutrophils) was measured in the blood of treated and untreated animals in the therapeutic study, finding equivalent levels in both. These findings indicate an equivalent amount of NETosis in both animals. However, the dosed cohort in the therapeutic trial had less surfactant protein-D (SP-D) in their blood. This indicates that the lung-blood barriers with the blood did not break down in the dosed animals. On the other hand, the vehicle-treated animals exhibited high levels of SP-D, demonstrating leakage of lung proteins into blood. This is consistent with the breakdown of alveolar/capillary surfaces.

The present study demonstrates that, in a non-limiting embodiment, digesting free and histone bound DNA with a potent DNA degrading biologic reduces frequency of or prevents death in DAH by addressing the underlying pathophysiology, e.g., activation of the STING pathway by free DNA in the blood and development of a Type-1 interferonopathy and cytokine storm. Under such situations, the acute immune response leads to the alveolar hemorrhage and death. The constructs described herein represent an immediate, non-toxic intervention in patients with DAH. As such, the constructs of the invention can be used to prevent death of DAH patients, reduce ICU time for patients with acute DAH, and/or decrease DAH morbidity.

DAH may be induced from several of underlying physiologic disruptions, all of which induce a severe immune reaction leading to so-called cytokine storm. As such, the constructs and methods of the disclosure can be used to treat patients suffering from DAH triggered by certain underlying conditions, such as but not limited to: ANCA vasculitis (antineutrophil cytoplasmic antibodies), Goodpasture's disease (antiglomerular basement membrane antibodies), lupus erythematosus (anti-nuclear antibodies and other serology), antiphospholipid syndrome (antiphospholipid antibodies), Lane-Hamilton Syndrome (antitransglutaminase or antiendomysial immunoglobulin A (IgA) antibodies), post-sepsis, bone marrow and organ transplant, exposure to cytotoxic and non-cytotoxic drugs, acute kidney injury, viral and/or bacterial infection, poststreptococcal glomerulonephritis or bacterial endocarditis (antistreptococcal antibodies or positive blood cultures), and/or SARS-CoV-2 infection (COVID-19).

The present disclosure provides stable and bioavailable constructs comprising DNAse1L3 and/or DNAse1 polypeptides (or fragments, rearrangements, (point) mutations, truncations, and/or any other modifications and/or analogues and/or derivatives thereof) fused with certain proteins. In certain embodiments, the constructs contemplated herein have increased bioavailability and/or developability over the DNAse1L3 and/or DNAse1 polypeptides known in the art. In certain embodiments, the constructs contemplated herein have enhanced enzymatic activity over the DNAse1L3 and/or DNAse1 polypeptides known in the art. In certain embodiments, the constructs contemplated herein have improved pharmacokinetic behavior over the DNAse1L3 and/or DNAse1 polypeptides known in the art. In certain embodiments, the constructs contemplated herein have enhanced stability over the DNAse1L3 and/or DNAse1 polypeptides known in the art.

In certain embodiments, the in vivo half-life of a construct of the disclosure is at least about 1.5, 2, 2.5, 3, 4, 5, 6, 7, 8, 9, 10, 12, 14, 16, 18, and/or 20 times higher than the DNAse1 and/or DNAse1L3 polypeptides described in the art. In other embodiments, the constructs of the disclosure are administered to the subject at a lower dose and/or at a lower frequency than other DNAse1 and/or DNAse1L3 polypeptides in the art. In yet other embodiments, the constructs of the disclosure are administered to the subject once a month, twice a month, three times a month, four times a month, once a week, or twice a week. In yet other embodiments, the lower frequency administration of the constructs of the disclosure results in better patient compliance and/or increased efficacy as compared with other DNAse1 and/or DNAse1L3 polypeptides in the art.

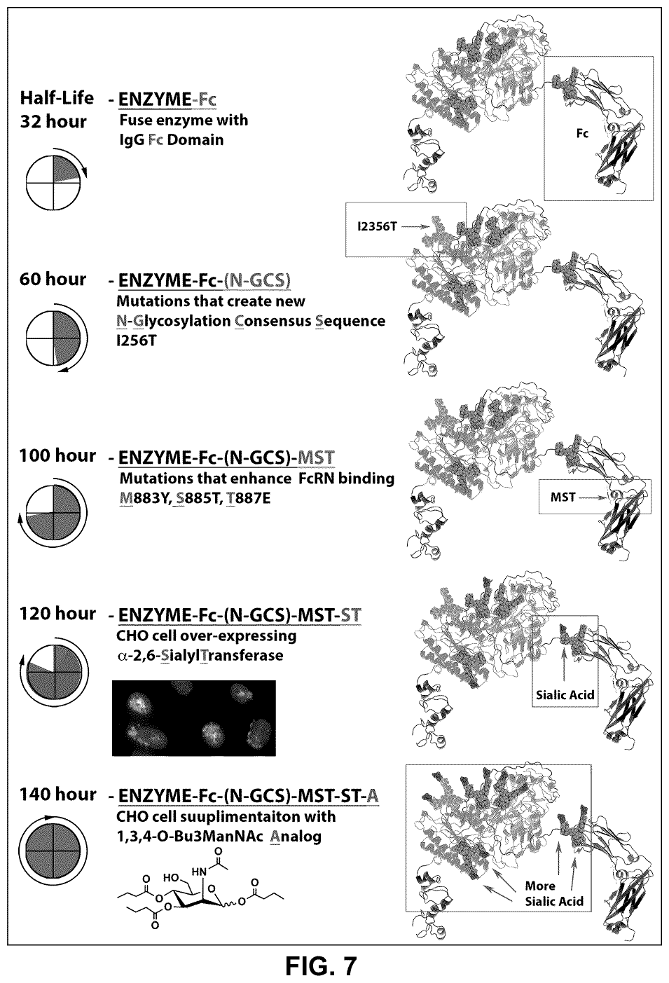

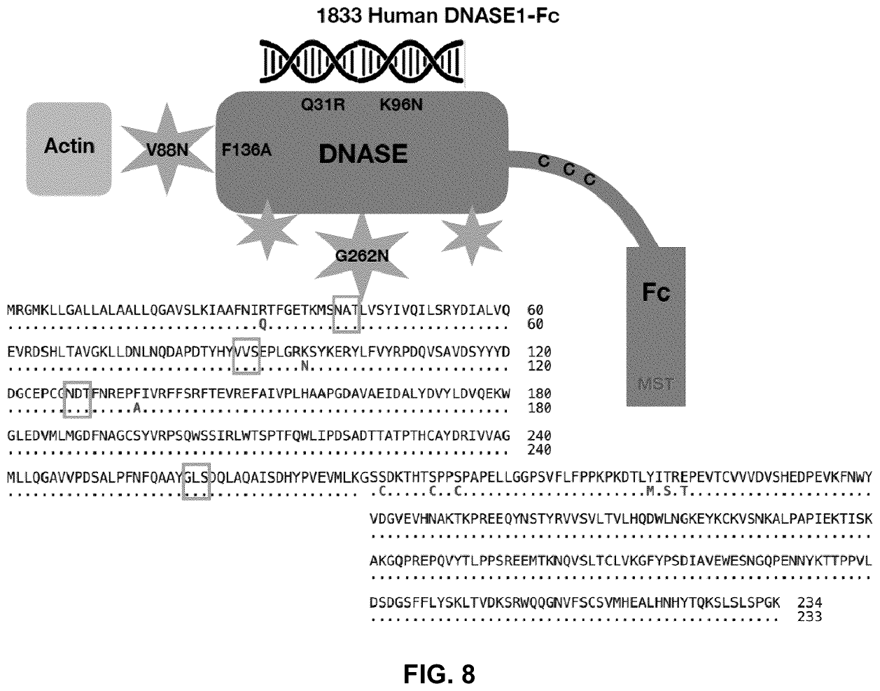

In one aspect, the present disclosure provides strategies for increasing the potency of enzyme biologics. In certain non-limiting embodiments, the present approach involves the stepwise improvement in the pharmacokinetic properties of a protein therapeutic by exploiting a suite of protein and glycosylation engineering methods. The approach is illustrated in a non-limiting manner in FIG. 7. In certain embodiments, the present disclosure contemplates adding one or more N-glycans to the protein and/or polypeptide. In certain embodiments, the present disclosure contemplates optimizing pH-dependent cellular recycling of the protein and/or polypeptide by protein engineering of the Fc neonatal receptor (FcRn). In certain embodiments, the present disclosure contemplates improving sialylation of the protein and/or polypeptide by first producing DNAse-Fc in cells stably transfected with human α-2,6-sialyltransferase (ST6). In certain embodiments, the present disclosure contemplates enhancing terminal sialylation of the protein and/or polypeptide by supplementing the production platform with 1,3,4-O-Bu₃ManNAc, sialic acid, and/or other precursors of sialic acid. Each of these steps can increased the area under the curve (AUC, a measure of in vivo drug availability) for the protein and/or polypeptide. In certain embodiments, this approach potentially extends once-a-day treatment to a monthly or bi-monthly dosing frequency.

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, separation science, and organic chemistry are those well-known and commonly employed in the art. The order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts, and premature terminations.

The term “antibody,” as used herein, refers to an immunoglobulin molecule that can specifically bind to a specific epitope on an antigen. Antibodies can be intact immunoglobulins derived from natural sources or from recombinant sources and can be immunoreactive portions of intact immunoglobulins.

As used herein, the term “AUC” refers to the area under the plasma drug concentration-time curve (AUC) and correlates with actual body exposure to drug after administration of a dose of the drug. In certain embodiments, the AUC is expressed in mg*h/L. The AUC can be used to measure bioavailability of a drug, which is the fraction of unchanged drug that is absorbed intact and reaches the site of action, or the systemic circulation following administration by any route.

AUC can be calculated used Linear Trapezoidal method or Logarithmic Trapezoidal method. The Linear Trapezoidal method uses linear interpolation between data points to calculate the AUC. This method, required by the OGD and FDA, is the standard for bioequivalence trials. For a given time interval (t₁−t₂), the AUC can be calculated as follows:

$$AUC = \frac{1}{2} ( {C}_{1} + {C}_{2} ) ( {t}_{2} - {t}_{1} )$$

wherein C₁ and C₂ are the average concentration over the time interval (t₁ and t₂).

The Logarithmic Trapezoidal method uses logarithmic interpolation between data points to calculate the AUC. This method is more accurate when concentrations are decreasing because drug elimination is exponential (which makes it linear on a logarithmic scale). For a given time interval (t₁−t₂), the AUC can be calculated as follows (assuming that C₁>C₂):

$$AUC = \frac{{C}_{1} - {C}_{2}}{ln ( {C}_{1} ) - ln ( {C}_{2} )} ( {t}_{2} - {t}_{1} )$$

The term “bioavailability” as used herein refers to the extent and rate at which the active moiety (protein or drug or metabolite) enters systemic circulation, thereby accessing the site of action, or the systemic circulation following administration by any route. Bioavailability of an active moiety is largely determined by the properties of the dosage form, which depend partly on its design and manufacture. Differences in bioavailability among formulations of a given drug or protein can have clinical significance; thus, knowing whether drug formulations are equivalent is essential. The most reliable measure of a drug's or protein's bioavailability is area under the plasma concentration-time curve (AUC). AUC is directly proportional to the total amount of unchanged drug or therapeutic protein that reaches systemic circulation. Drug or therapeutic protein may be considered bioequivalent in extent and rate of absorption if their plasma concentration curves are essentially superimposable. For an intravenous dose of a drug, bioavailability is defined as unity. For drug administered by other routes of administration, bioavailability is often less than unity. Incomplete bioavailability may be due to a number of factors that can be subdivided into categories of dosage form effects, membrane effects, and site of administration effect. Half-life and AUC provide information about the bioavailability of a drug or biologic.

As used herein, the terms “conservative variation” or “conservative substitution” as used herein refers to the replacement of an amino acid residue by another, biologically similar residue. Conservative variations or substitutions are not likely to change the shape of the peptide chain. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine.

As used herein, a “construct” of the disclosure refers to a fusion polypeptide comprising a DNAse1 and/or DNAse1L3 polypeptide, or any fragments, rearrangements, (point) mutations, truncations, or any other modifications and/or analogues and/or derivatives thereof.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “DNAse1” refers to deoxyribonuclease-1 (UniProtKB=P24855). The sequence of human DNAse1 is provided herein (SEQ ID NO:1). In certain embodiments, the signal peptide of DNAse1 corresponds to residues 1-22 of SEQ ID NO:1.

```
SEQ ID NO: 1
                10 20 30 40 50
MRGMKLLGAL LALAALLQGA VSLKIAAFNI QTFGETKMSN ATLVSYIVQI
                60 70 80 90 100
LSRYDIALVQ EVRDSHLTAV GKLLDNLNQD APDTYHYVVS EPLGRNSYKE
              110 120 130 140 150
RYLFVYRPDQ VSAVDSYYYD DGCEPCGNDT FNREPAIVRE FSRFTEVREF
              160 170 180 190 200
AIVPLHAAPG DAVAEIDALY DVYLDVQEKW GLEDVMLMGD FNAGCSYVRP
              210 220 230 240 250
SQWSSIRLWT SPTFQWLIPD SADTTATPTH CAYDRIVVAG MLLRGAVVPD
              260 270 280
SALPENFQAA YGLSDOLAQA ISDHYPVEVM LK
The sequence of mouse DNAse1 is provided herein (SEQ ID NO: 29):
MRYTGLMGTLLTLVNLLQLAGTLRIAAFNIRTFGETKMSNATLSVYFVKILSRYDIAVIQEVRDSHLVAV
GKLLDELNRDKPDTYRYVVSEPLGRKSYKEQYLFVYRPDQVSILDSYQYDDGCEPCGNDTFSREPAIVKF
FSPYTEVQEFAIVPLHAAPTEAVSEIDALYDVYLDVWQKWGLEDIMEMGDFNAGCSYVTSSQWSSIRLRT
SPIFQWLIPDSADTTVTSTHCAYDRIVVAGALLQAAVVPNSAVPEDFQAEYGLSNQLAEAISDHYPVEVT
LRKI
```

The sequence alignment of human DNAse1 (SEQ ID NO:1, sequence ‘1’ below) and mouse DNAse1 (SEQ ID NO:29, sequence ‘2’ below) follows:

DNAse1

| | | |
|---|---|---|
| 1 | MRGMKLLGALLALAALLQGAVSLKIAAFNIQTFGETKMSNATLVSYIVQILSRYDIALVQ | 60 |
| 2 | MRYTGLMGTLLTLVNLLQLAGTLRIAAFNIRTFGETKMSNATLSVYFVKILSRYDIAVIQ | 60 |
| | ** *:*:**:*. *** * :*:******:************ *:*:********::* | |
| 1 | EVRDSHLTAVGKLLDNLNQDAPDTYHYVVSEPLGRNSYKERYLFVYRPDQVSAVDSYYYD | 120 |
| 2 | EVRDSHLVAVGKLLDELNRDKPDTYRYVVSEPLGRKSYKEQYLFVYRPDQVSILDSYQYD | 120 |
| | *******.*******:**:* ****:*********:****:*********** :*** ** | |
| 1 | DGCEPCGNDTFNREPAIVRFFSRFTEVREFAIVPLHAAPGDAVAEIDALYDVYLDVQEKW | 180 |
| 2 | DGCEPCGNDTFSREPAIVKFFSPYTEVQEFAIVPLHAAPTEAVSEIDALYDVYLDVWQKW | 179 |
| | ***********.******:*** :***:*********** :**:************ :** | |
| 1 | GLEDVMLMGDFNAGCSYVRPSQWSSIRLWTSPTFQWLIPDSADTTATPTHCAYDRIVVAG | 240 |
| 2 | GLEDIMFMGDFNAGCSYVTSSQWSSIRLRTSPIFQWLIPDSADTTVTSTHCAYDRIVVAG | 239 |
| | ****:*:*********** ******** *** ************.* ************ | |
| 1 | MLLRGAVVPDSALPFNFQAAYGLSDQLAQAISDHYPVEVMLK-- | 282 |
| 2 | ALLQAAVVPNSAVPFDFQAEYGLSNQLAEAISDHYPVEVTLRKI | 283 |
| | **:.****:**:**:*** ****:***:********** *: | |

As used herein, “human DNAse1” refers to the human DNAse1 sequence as described herein, or any fragments, rearrangements, (point) mutations, truncations, or any other modifications and/or analogues and/or derivatives thereof. As used herein, the term “enzymatically active” with respect to DNAse1 is defined as being capable of binding and hydrolyzing DNA.

As used herein, the term “DNAse1L3” refers to deoxyribonuclease gamma (UniProtKB=Q13609). The sequence of human DNAse1L3 is provided herein (SEQ ID NO:2). In certain embodiments, the signal peptide of DNAse1L3 corresponds to residues 1-20 of SEQ ID NO:2. In certain embodiments, the nuclear localization signal of DNAse1L3 corresponds to residues 296-304 of SEQ ID NO:2. In certain embodiments, the nuclear localization signal of DNAse1L3 corresponds to residues 292-304 of SEQ ID NO:2. In certain embodiments, the nuclear localization signal of DNAse1L3 corresponds to residues 291-305 of SEQ ID NO:2. In certain embodiments, the nuclear localization signal of DNAse1L3 corresponds to residues A-B of SEQ ID NO:2, wherein A ranges from 291 to 296 and B ranges from 304 to 305.

```
SEQ ID NO: 2
                10 20 30 40
MSRELAPLLL LLLSIHSALA MRICSFNVRS FGESKQEDKN
                50 60 70 80
AMDVIVKVIK RCDIILVMEI KDSNNRICPI LMEKLNRNSR
                90 100 110 120
RGITYNYVIS SRLGRNTYKE QYAFLYKEKL VSVKRSYHYH
              130 140 150 160
DYQDGDADVF SREPFVVWFQ SPHTAVKDFV IIPLHTTPET
              170 180 190 200
SVKEIDELVE VYTDVKHRWK AENFIFMGDF NAGCSYVPKK
              210 220 230 240
AWKNIRLRTD PRFVWLIGDQ EDTTVKKSTN CAYDRIVLRG
              250 260 270 280
QEIVSSVVPK SNSVFDFQKA YKLTEEEALD VSDHFPVEFK
              290 300
LQSSRAFTNS KKSVTLRKKT KSKRS
The sequence of mouse DNAse1L3 is provided
herein (SEQ ID NO: 30):
MSLHPASPRLASLLLFILALHDTLALRLCSFNVRSFGASKKEN
HEAMDIIVKIIKRCDLILLMEIKDSSNNICPMLMEKLNGNSRR
STTYNYVISSRLGRNTYKEQYAFVYKEKLVSVKTKYHYHDYQD
GDTDVFSREPFVVWFHSPFTAVKDFVIVPLHTTPETSVKEIDE
LVDVYTDVRSQWKTENFIFMGDFNAGCSYVPKKAWQNIRLRTD
PKFVWLIGDQEDTTVKKSTSCAYDRIVLCGQEIVNSVVPRSSG
VFDFQKAYDLSEEEALDVSDHFPVEFKLQSSRAFTNNRKSVSL
KKRKKGNRS
```

The sequence alignment of human DNAse1L3 (SEQ ID NO:2, sequence ‘1’ below) and mouse DNAse1L3 (SEQ ID NO:30, sequence ‘2’ below) follows:

```
DNAse1L3
1 -----MSRELAPLLLLLLSIHSALAMRICSFNVRSFGESKQEDKNAMDVIVKVIKRCDII 55
2 MSLHPASPRLASLLLFILALHDTLALRLCSFNVRSFGASKKENHEAMDIIVKIIKRCDLI 60
                * .** ***::*::*.:**:*:********* **:*:::***:***:*****:*
1 LVMEIKDSNNRICPILMEKLNRNSRRGITYNYVISSRLGRKTYKEQYAFLYKEKLVSVKR 115
2 LLMEIKDSSNNICPMLMEKLNGNSRRSTTYNYVISSRLGRNTYKEQYAFVYKEKLVSVKT 120
    *:******.*.***:****** ****. ************:********:*********
1 SYHYHDYQDGDADVFSREPFVVWFQSPHTAVKDFVIIPLHTTPETSVKEIDELVEVYTDV 175
2 KYHYHDYQDGDTDVFSREPFVVWFHSPFTAVKDFVIVPLHTTPETSVKEIDELVDVYTDV 180
    .**********:************:**.********:*****************:*****
1 KHRWKAENFIFMGDFNAGCSYVPKKAWKNIRLRTDPRFVWLIGDQEDTTVKKSTNCAYDR 235
2 RSQWKTENFIFMGDFNAGCSYVPKKAWQNIRLRTDPKFVWLIGDQEDTTVKKSTSCAYDR 240
    : :**:*********************:********:*****************:*****
1 IVLRGQEIVSSVVPKSNSVFDFQKAYKLTEEEALDVSDHFPVEFKLQSSRAFTNSKKSVT 295
2 IVLCGQEIVNSVVPRSSGVFDFQKAYDLSEEEALDVSDHFPVEFKLQSSRAFTNNRKSVS 300
    *** *****.****:*..********.*:*************************.:***:
1 LRKKTKSKRS 305
2 LKKRKKGNRS 310
    *:*:.*.:**
```

As used herein, “human DNAse1L3” refers to the human DNAse1L3 sequence as described herein, or any fragments, rearrangements, (point) mutations, truncations, or any other modifications and/or analogues and/or derivatives thereof. As used herein, the term “enzymatically active” with respect to DNAse1L3 is defined as being capable of binding and hydrolyzing DNA.

As used herein, the term “DNAse1-Fc” refers to a DNAse1 polypeptide recombinantly fused and/or chemically conjugated (including both covalent and non-covalent conjugations) to an FcR binding domain of an IgG molecule (preferably, a human IgG). In certain embodiments, the C-terminus of DNAse1 is fused or conjugated to the N-terminus of the FcR binding domain. In certain embodiments, the N-terminus of DNAse1 is fused or conjugated to the C-terminus of the FcR binding domain.

As used herein, the term “DNAse1L3-Fc” refers to a DNAse1L3 polypeptide recombinantly fused and/or chemically conjugated (including both covalent and non-covalent conjugations) to an FcR binding domain of an IgG molecule (preferably, a human IgG). In certain embodiments, the C-terminus of DNAse1L3 is fused or conjugated to the N-terminus of the FcR binding domain. In certain embodiments, the N-terminus of DNAse1L3 is fused or conjugated to the C-terminus of the FcR binding domain.

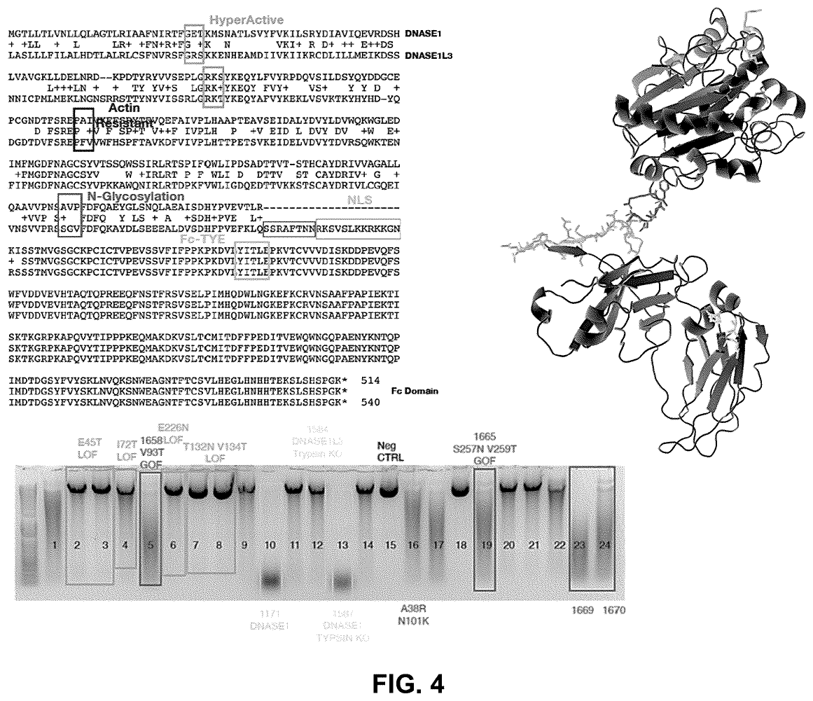

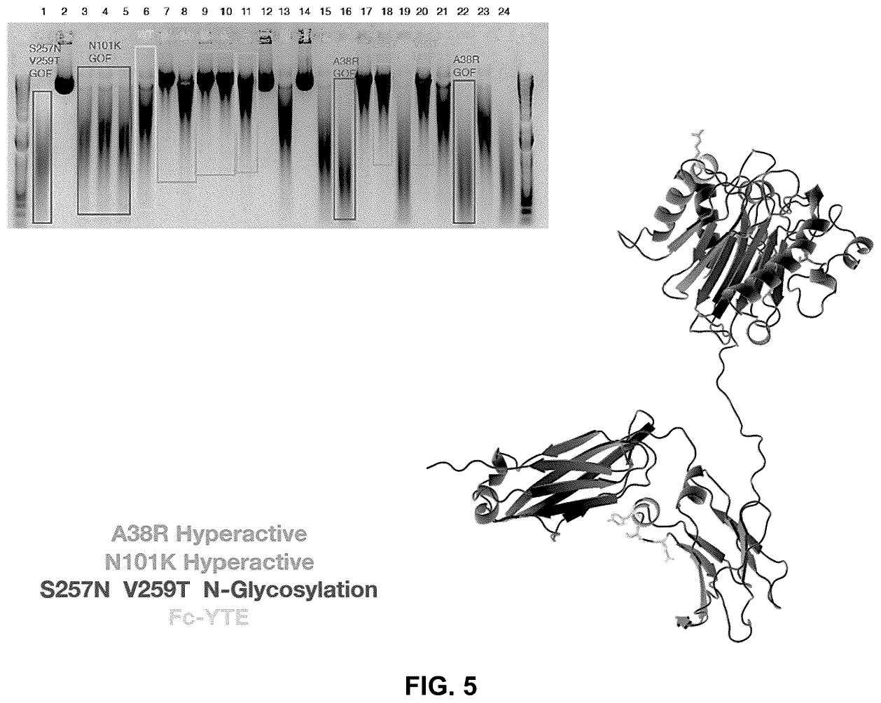

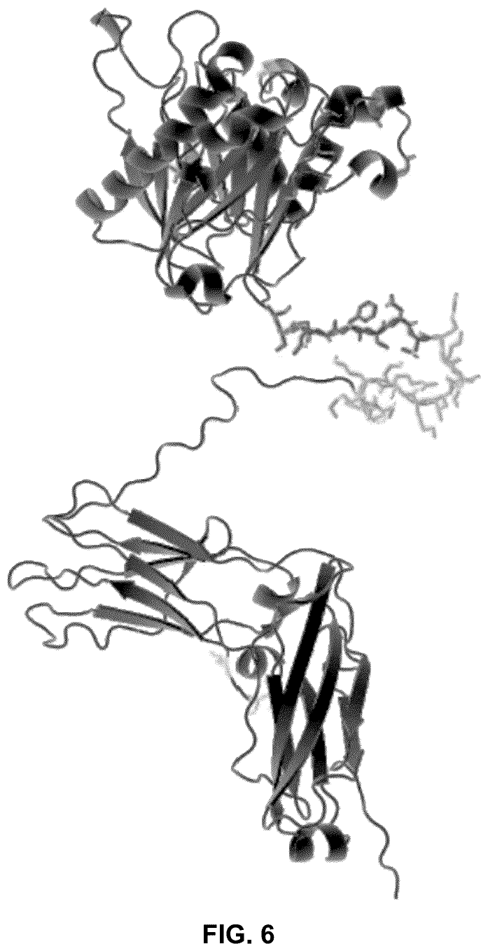

The sequence alignment of mouse DNAse 1 (SEQ ID NO:42, ‘query’ below) and mouse DNAse1L3 (SEQ ID NO:43, ‘Sbjct’ below), as shown in FIG. 4 herein, follows:

| | | | |
|---|---|---|---|
| Query | 7 | MGTLLTLVNLLQLAGTLRIAAFNIRTFGETKMSNATLSVYFVKILSRYDIAVIQEVRDSH | 66 |
| | | + +LL + L LR+ +FN+R+FG +K N VKI+ R D+ ++ E++DS | |
| Sbjct | 10 | LASLLLFILALHDTLALRLCSFNVRSFGRSKKENHEAMDIIVKIIKRCDLILLMEIKDSS | 69 |
| Query | 67 | LVAVGKLLDELNRD--KPDTYRYVVSEPLGRKSYKEQYLFVYRPDQVSILDSYQYDDGCE | 124 |
| | | L+++LN + + TY YV+S LGRK+YKEQY FVY+ VS+ Y Y D + | |
| Sbjct | 70 | NNICPMLMEKLNGNSRRSTTYNYVISSRLGRKTYKEQYAFVYKEKLVSVKTKYHYHD-YQ | 128 |
| Query | 125 | PCGNDTFSREPAIVKFFSPYTEVQEFAIVPLHAAPTEAVSEIDALYDVYLDVWQKWGLED | 184 |
| | | D FSREP +V F SP+T V++F IVPLH P +V EID L DVY DV +W E+ | |
| Sbjct | 129 | DGDTDVFSREPFVVWFHSPFTAVKDFVIVPLHTTPETSVKEIDELVDVYTDVRSQWKTEN | 188 |
| Query | 185 | IMFMGDFNAGCSYVTSSQWSSIRLRTSPIFQWLIPDSADTTVT-STHCAYDRIVVAGALL | 243 |
| | | +FMGDENAGCSYV W +IRLRT P F WLI D DTTV ST CAYDRIV+ G + | |
| Sbjct | 189 | FIFMGDFNAGCSYVPKKAWQNIRLRTDPKFVWLIGDQEDTTVKKSTSCAYDRIVLCGQEI | 248 |
| Query | 244 | QAAVVPNSAVPFDFQAEYGLSNQLAEAISDHYPVEVTLR--------------------- | 282 |
| | | +VVP S+ FDFQ Y LS + A +SDH+PVE L+ | |
| Sbjct | 249 | VNSVVPRSSGVFDFQKAYDLSEEEALDVSDHFPVEFKLQSSRAFTNNRKSVSLKKRKKGN | 308 |
| Query | 283 | KISSTMVGSGCKPCICTVPEVSSVFIFPPKPKDVLYITLEPKVTCVVVDISKDDPEVQFS | 342 |
| | | + SSTMVGSGCKPCICTVPEVSSVFIFPPKPKDVLYITLEPKVTCVVVDISKDDPEVQFS | |
| Sbjct | 309 | RSSSTMVGSGCKPCICTVPEVSSVFIFPPKPKDVLYITLEPKVTCVVVDISKDDPEVQFS | 368 |
| Query | 343 | WFVDDVEVHTAQTQPREEQFNSTFRSVSELPIMHQDWLNGKEFKCRVNSAAFPAPIEKTI | 402 |
| | | WFVDDVEVHTAQTQPREEQFNSTFRSVSELPIMHQDWLNGKEFKCRVNSAAFPAPIEKTI | |
| Sbjct | 369 | WFVDDVEVHTAQTQPREEQFNSTFRSVSELPIMHQDWLNGKEFKCRVNSAAFPAPIEKTI | 428 |
| Query | 403 | SKTKGRPKAPQVYTIPPPKEQMAKDKVSLTCMITDFFPEDITVEWQWNGQPAENYKNTQP | 462 |
| | | SKTKGRPKAPQVYTIPPPKEQMAKDKVSLTCMITDFFPEDITVEWQWNGQPAENYKNTQP | |
| Sbjct | 429 | SKTKGRPKAPQVYTIPPPKEQMAKDKVSLTCMITDFFPEDITVEWQWNGQPAENYKNTQP | 488 |
| Query | 463 | IMDTDGSYFVYSKLNVQKSNWEAGNTFTCSVLHEGLHNHHTEKSLSHSPGK* | 514 |
| | | IMDTDGSYFVYSKLNVQKSNWEAGNTFTCSVLHEGLHNHHTEKSLSHSPGK* | |
| Sbjct | 489 | IMDTDGSYFVYSKLNVQKSNWEAGNTFTCSVLHEGLHNHHTEKSLSHSPGK* | 540 |

As used herein, the term “Fc” refers to a human IgG (immunoglobulin) Fc domain. Subtypes of IgG such as IgG1, IgG2, IgG3, and IgG4 are contemplated for usage as Fc domains. As used herein, the term “IgG1-IgG4” refers to IgG1, IgG2, IgG3, and/or IgG4, and/or any combinations thereof.

As used herein, the “Fe region” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “Fc receptors” refer to proteins found on the surface of certain cells (including, among others, B lymphocytes, follicular dendritic cells, natural killer cells, macrophages, neutrophils, eosinophils, basophils, human platelets, and mast cells) that contribute to the protective functions of the immune system. Fc receptors bind to antibodies that are attached to infected cells or invading pathogens. Immunoglobulin Fc receptors (FcRs) are expressed on all hematopoietic cells and play crucial roles in antibody-mediated immune responses. Binding of immune complexes to FcR activates effector cells, leading to phagocytosis, endocytosis of IgG-opsonized particles, releases of inflammatory mediators, and antibody-dependent cellular cytotoxicity (ADCC). Fc receptors have been described for all classes of immunoglobulins: FcγR and neonatal FcR (FcRn) for IgG, FcεR for IgE, FcαR for IgA, FcδR for IgD and Fc R for IgM. All known Fc receptors structurally belong to the immunoglobulin superfamily, except for FcRn and FcεRII, which are structurally related to class I Major Histocompatibility antigens and C-type lectins, respectively (Fc Receptors, Neil A. Fangera, et al., in Encyclopedia of Immunology (2^{nd} Edition), 1998).

As used herein, the term “FcRn Receptor” refers to the neonatal Fc receptor (FcRn), also known as the Brambell receptor, which is a protein that in humans is encoded by the FCGRT gene. An FcRn specifically binds the Fc domain of an antibody. FcRn extends the half-life of IgG and serum albumin by reducing lysosomal degradation in endothelial cells. IgG, serum albumin, and other serum proteins are continuously internalized through pinocytosis. Generally, serum proteins are transported from the endosomes to the lysosome, where they are degraded. FcRn-mediated transcytosis of IgG across epithelial cells is possible because FcRn binds IgG at acidic pH (<6.5) but not at neutral or higher pH. IgG and serum albumin are bound by FcRn at the slightly acidic pH (<6.5) and recycled to the cell surface where they are released at the neutral pH (>7.0) of blood. In this way IgG and serum albumin avoid lysosomal degradation.

The Fc portion of an IgG molecule is located in the constant region of the heavy chain, notably in the CH2 domain. The Fc region binds to an Fc receptor (FcRn), which is a surface receptor of a B cell and also proteins of the complement system. The binding of the Fc region of an IgG molecule to an FcRn activates the cell bearing the receptor and thus activates the immune system. The Fc residues critical to the mouse Fc-mouse FcRn and human Fc-human FcRn interactions have been identified (Dall'Acqua et al., 2002, J. Immunol. 169(9):5171-80). An FcRn binding domain comprises the CH2 domain (or a FcRn binding portion thereof) of an IgG molecule.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

The term “functional equivalent” or “functional derivative” denotes, in the context of a functional derivative of an amino acid sequence, a molecule that retains a biological activity (either function or structural) substantially similar to that of sequences of DNAse1-Fc and/or DNAse1E3-Fc constructs shown herein. A functional derivative or equivalent may be a natural derivative or is prepared synthetically. The functionally equivalent polypeptides of the disclosure can also be polypeptides identified using one or more techniques of structural and or sequence alignment known in art.

Exemplary functional derivatives include amino acid sequences having substitutions, deletions, or additions of one or more amino acids, provided that the biological activity of the protein is conserved. The substituting amino acid desirably has chemico-physical properties which are similar to that of the substituted amino acid. Desirable similar chemico-physical properties include similarities in charge, bulkiness, hydrophobicity, hydrophilicity, and the like. Typically, greater than 30% identity between two polypeptides is an indication of functional equivalence. Preferably, functionally equivalent polypeptides of the disclosure have a degree of sequence identity with the DNAse1-Fc and/or DNAse1L3-Fc constructs of greater than 80%. More preferred polypeptides have degrees of identity of greater than 85%, 90%, 95%, 98% or 99%, respectively. Method for determining whether a functional equivalent or functional derivative has the same or similar or higher biological activity than the DNAse1-Fc and/or DNAse1L3-Fc construct can be determined by using enzymology assays known in the art.

“Gene transfer” and “gene delivery” refer to methods or systems for reliably inserting a particular nucleic acid sequence into targeted cells.

An “inducible” promoter is a nucleotide sequence that, when operably linked with a polynucleotide that encodes or specifies a gene product, causes the gene product to be produced in a cell substantially only when an inducer that corresponds to the promoter is present in the cell.

As used herein, the term “in vivo half-life” for a protein and/or polypeptide contemplated within the disclosure (such as, for example, a DNAse1 and/or DNAse1L3 construct containing FcRn binding sites) refers to the time required for half the quantity administered in the animal to be cleared from the circulation and/or other tissues in the animal. When a clearance curve of a fusion protein is constructed as a function of time, the curve is usually biphasic with a rapid α-phase (which represents an equilibration of the administered molecules between the intra- and extra-vascular space and which is, in part, determined by the size of molecules), and a longer β-phase (which represents the catabolism of the molecules in the intravascular space). In certain embodiments, the term “in vivo half-life” in practice corresponds to the half-life of the molecules in the β-phase.

“Instructional material,” as that term is used herein, includes a publication, a recording, a diagram, or any other medium of expression that can be used to communicate the usefulness of the nucleic acid, peptide, and/or compound of the disclosure in the kit for identifying or alleviating or treating the various diseases or disorders recited herein.

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences that are normally adjacent to the fragment, i.e., the sequences adjacent to the fragment in a genome in which it naturally occurs. The term also applies to nucleic acids that have been substantially purified from other components which naturally accompany the nucleic acid, i.e., RNA or DNA or proteins, which naturally accompany it in the cell. The term therefore includes, for example, a recombinant DNA that is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA that is part of a hybrid gene encoding additional polypeptide sequence.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “patient,” “individual” or “subject” refers to a human.

As used herein, the term “pharmaceutical composition” or “composition” refers to a mixture of at least one compound useful within the disclosure with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent, or encapsulating material, involved in carrying or transporting a compound useful within the disclosure within or to the patient such that it may perform its intended function. Each carrier must be “acceptable” in the sense of being compatible with the other ingredients of the formulation, including the compound useful within the disclosure, and not injurious to the patient. Some examples of materials that may serve as pharmaceutically acceptable carriers include sugars, such as lactose, glucose and sucrose; starches, such as corn starch and potato starch; cellulose, and its derivatives. As used herein, “pharmaceutically acceptable carrier” also includes any and all coatings, antibacterial and antifungal agents, and absorption delaying agents, and the like that are compatible with the activity of the compound useful within the disclosure and are physiologically acceptable to the patient. The “pharmaceutically acceptable carrier” may further include a pharmaceutically acceptable salt of the compound useful within the disclosure. Other additional ingredients that may be included in the pharmaceutical compositions used in the practice of the disclosure are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “polypeptide” refers to a polymer composed of amino acid residues, related naturally occurring structural variants, and synthetic non-naturally occurring analogues thereof linked via peptide bonds.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements that are required for expression of the gene product. The promoter/regulatory sequence may for example be one that expresses the gene product in a tissue specific manner.

The term “recombinant polypeptide” as used herein is defined as a polypeptide produced by using recombinant DNA methods.

The term “recombinant DNA” as used herein is defined as DNA produced by joining pieces of DNA from different sources.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, the term “signal peptide” refers to a sequence of amino acid residues (ranging in length from, for example, 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish, et al., 2000, Molecular Cell Biology, 4^{th} edition).

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

A “tissue-specific” promoter is a nucleotide sequence that, when operably linked with a polynucleotide encodes or specified by a gene, causes the gene product to be produced in a cell substantially only if the cell is a cell of the tissue type corresponding to the promoter.

The phrase “under transcriptional control” or “operatively linked” as used herein means that the promoter is in the correct location and orientation in relation to a polynucleotide to control the initiation of transcription by RNA polymerase and expression of the polynucleotide.

The term “transfected” or “transformed” or “transduced” as used herein refers to a process by which exogenous nucleic acid is transferred or introduced into the host cell. A “transfected” or “transformed” or “transduced” cell has been transfected, transformed or transduced with exogenous nucleic acid. The cell includes the primary subject cell and its progeny.

As used herein, the term “treatment” or “treating” is defined as the application or administration of a therapeutic agent, i.e., a compound useful within the disclosure (alone or in combination with another pharmaceutical agent), to a patient, or application or administration of a therapeutic agent to an isolated tissue or cell line from a patient (e.g., for diagnosis or ex vivo applications), who has a disease or disorder, or a symptom of a disease or disorder, with the purpose to cure, heal, alleviate, relieve, alter, remedy, ameliorate, improve or affect the disease or disorder, or the symptoms of the disease or disorder. Such treatments may be specifically tailored or modified, based on knowledge obtained from the field of pharmacogenomics.

“Variant” as the term is used herein, is a nucleic acid sequence or a peptide sequence that differs in sequence from a reference nucleic acid sequence or peptide sequence respectively but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide may differ in amino acid sequence by one or more substitutions, additions, or deletions in any combination. A variant of a nucleic acid or peptide may be a naturally occurring such as an allelic variant, or may be a variant that is not known to occur naturally. Non-naturally occurring variants of nucleic acids and peptides may be made by mutagenesis techniques or by direct synthesis.

A “vector” is a composition of matter that comprises an isolated nucleic acid and that may be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

As used herein, the term “virus” is defined as a particle consisting of nucleic acid (RNA or DNA) enclosed in a protein coat, with or without an outer lipid envelope, which is capable of transfecting the cell with its nucleic acid.

As used herein, the term “wild-type” refers to a gene or gene product isolated from a naturally occurring source. A wild-type gene is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Constructs and Polypeptides

In one aspect, the disclosure provides a DNAse1-Fc and/or DNAse1L3-Fc construct. The disclosure contemplates that the constructs contemplated herein can have one or more of the mutations described herein.

Further, the disclosure provides homodimeric constructs comprising two independently selected DNAse1 constructs of the disclosure. Further, the disclosure provides homodimeric constructs comprising two independently selected DNAse1L3 constructs of the disclosure. Further, the disclosure provides heterodimeric constructs comprising a DNAse1 construct of the disclosure and a DNAse1L3 construct of the disclosure.

The disclosure provides the constructs described herein, as well as any glycosylation variants (alternative glycoforms), as well as constructs that have been modified through site-directed mutagenesis or any sort of protein chemistry manipulation so as to have improved solubility and/or enzymatic activity and/or in vivo half-life.

In certain embodiments, the construct comprises the amino acid sequence:

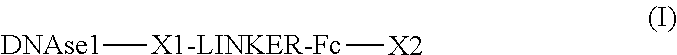

wherein:
DNAse1 is a human DNAse1 polypeptide as described elsewhere herein;
X1 is a covalent bond, or X1 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1-IgG4 as described elsewhere herein.

In certain embodiments, (I) describes the construct from left to right as from its N-terminus to its C-terminus. In that case, the N-terminus of the Fc is linked to the C-terminus of the DNAse1. In certain embodiments, (I) describes the construct from left to right as from its C-terminus to its N-terminus. In that case, the C-terminus of the Fe is linked to the N-terminus of the DNAse1.

In certain embodiments, the construct comprises the amino acid sequence:

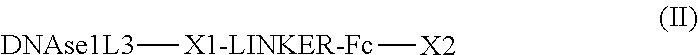

wherein:
DNAse1L3 is a human polypeptide DNAse1L3 as described elsewhere herein;
X1 is a covalent bond, or X1 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof;
LINKER is a covalent bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1-IgG4 as described elsewhere herein.

In certain embodiments, (II) describes the construct from left to right as from its N-terminus to its C-terminus. In that case, the N-terminus of the Fe is linked to the C-terminus of the DNAse1L3. In certain embodiments, (II) describes the construct from left to right as from its C-terminus to its N-terminus. In that case, the C-terminus of the Fe is linked to the N-terminus of the DNAse1L3.

In certain embodiments, the construct comprises the amino acid sequence:

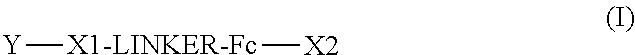

wherein:
Y is a human DNAse1 polypeptide or a human DNAse1L3 polypeptide;
X1 is RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1-IgG4.

### Fc:

In certain embodiments, the Fc domain of human
IgG1 has the following sequence:
SEQ ID NO: 4: hIgG1 Fc domain, Fc (human)
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPGK
SEQ ID NO: 45: hIgG1 Fc domain, Fc (human)
CDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK
In certain embodiments, the Fc domain of mouse
IgG1 has the following sequence:
SEQ ID NO: 31: hIgG1 Fc domain, Fc (mouse)
GCKPCICTVPEVSSVFIFPPKPKDVLYITLEPKVTCVVVDISKDDPEVQF
SWFVDDVEVHTAQTQPREEQFNSTFRSVSELPIMHQDWLNGKEFKCRVNS
AAFPAPIEKTISKTKGRPKAPQVYTIPPPKEQMAKDKVSLTCMITDFFPE
DITVEWQWNGQPAENYKNTQPIMDTDGSYFVYSKLNVQKSNWEAGNTFTC
SVLHEGLHNHHTEKSLSHSPGK

In certain embodiments, Cys6 (C6) with respect to SEQ ID NO:4 is mutated to another amino acid, such as but not limited to Gly or Ser. In certain embodiments, Cys9 (C9) with respect to SEQ ID NO:4 is mutated to another amino acid, such as but not limited to Gly or Ser. In a non-limiting embodiment, any one of such mutations in the C6/C9 residues responsible for the interchain disulfide bond in the heavy chain of the Fc domain converts a dimeric enzyme fusion to a monomeric fusion, thus allowing for greater accessibility to chromatin and microparticle DNA.

In certain embodiments, Cys1 (C1) with respect to SEQ ID NO:45 is mutated to another amino acid, such as but not limited to Gly or Ser. In certain embodiments, Cys7 (C7) with respect to SEQ ID NO:45 is mutated to another amino acid, such as but not limited to Gly or Ser. In certain embodiments, Cys10 (C10) with respect to SEQ ID NO:45 is mutated to another amino acid, such as but not limited to Gly or Ser. In a non-limiting embodiment, any one of such mutations in the C7/C10 residues responsible for the interchain disulfide bond in the heavy chain of the Fc domain converts a dimeric enzyme fusion to a monomeric fusion, thus allowing for greater accessibility to chromatin and microparticle DNA.

In certain embodiments, the hIgG1 Fc domain has at least one of the following mutations with respect to SEQ ID NO:4: M32Y, S34T, and T36E. In a non-limiting embodiment, any such mutation enhances endosomal recycling of the corresponding construct. In certain embodiments, the hIgG Fc domain has the following mutations with respect to SEQ ID NO:4: M32Y, S34T, and T36E.

A non-limiting list of contemplated mutations in the Fc domain of the constructs of the disclosure include C6S, C9S, M32Y, S34T, and/or T36E with respect to SEQ ID NO:4. In certain embodiments, the Fc domain of the construct comprise the C6S mutation with respect to SEQ ID NO:4. In certain embodiments, the Fc domain of the construct comprise the C9S mutation with respect to SEQ ID NO:4. In certain embodiments, the Fc domain of the construct comprise the M32Y mutation with respect to SEQ ID NO:4. In certain embodiments, the Fc domain of the construct comprise the S34T mutation with respect to SEQ ID NO:4. In certain embodiments, the Fc domain of the construct comprise the T36E mutation with respect to SEQ ID NO:4.

In certain embodiments, the hIgG1 Fc domain has at least one of the following mutations with respect to SEQ ID NO:45: M33Y, S35T, and T37E. In a non-limiting embodiment, any such mutations enhance endosomal recycling of the corresponding construct. In certain embodiments, the hIgG Fc domain has the following mutations with respect to SEQ ID NO:45: M33Y, S35T, and T37E.

A non-limiting list of contemplated mutations in the Fc domain of the constructs of the disclosure include C1S, C7S, C10S, M33Y, S35T, and/or T37E with respect to SEQ ID NO:45. In certain embodiments, the Fc domain of the construct comprise the C1S mutation with respect to SEQ ID NO:45. In certain embodiments, the Fc domain of the construct comprise the C7S mutation with respect to SEQ ID NO:45. In certain embodiments, the Fc domain of the construct comprise the C10S mutation with respect to SEQ ID NO:45. In certain embodiments, the Fc domain of the construct comprise the M33Y mutation with respect to SEQ ID NO:45. In certain embodiments, the Fc domain of the construct comprise the S35T mutation with respect to SEQ ID NO:45. In certain embodiments, the Fc domain of the construct comprise the T37E mutation with respect to SEQ ID NO:45.

### Linker:

In certain embodiments, the LINKER is a chemical bond or absent. In certain embodiments, the LINKER is a polypeptide comprising 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, and/or 1 amino acid(s). In certain embodiments, the LINKER comprises Gly and/or Ser amino acids.

In certain embodiments, the LINKER is/comprises Ala (A). In certain embodiments, the LINKER is/comprises Cys (C). In certain embodiments, the LINKER is/comprises Asp (D). In certain embodiments, the LINKER is/comprises Glu (E). In certain embodiments, the LINKER is/comprises Phe (F). In certain embodiments, the LINKER is/comprises Gly (G). In certain embodiments, the LINKER is/comprises His (H). In certain embodiments, the LINKER is/comprises Ile (I). In certain embodiments, the LINKER is/comprises Lys (K). In certain embodiments, the LINKER is/comprises Leu (L). In certain embodiments, the LINKER is/comprises Met (M). In certain embodiments, the LINKER is/comprises Asn (N). In certain embodiments, the LINKER is/comprises Pro (P). In certain embodiments, the LINKER is/comprises Gln (Q). In certain embodiments, the LINKER is/comprises Arg (R). In certain embodiments, the LINKER is/comprises Ser (S). In certain embodiments, the LINKER is/comprises Thr (T). In certain embodiments, the LINKER is/comprises Val (V). In certain embodiments, the LINKER is/comprises Tyr (Y). In certain embodiments, the LINKER is/comprises Trp (W).

In certain embodiments, the LINKER is a single amino acid.

In certain embodiments, the LINKER is a dipeptide. In certain embodiments, the first amino acid of the dipeptide is selected from the group consisting of A, C, D, E, F, G, H, I, K, L, M, N, P, Q, R, S, T, V, Y, and W. In certain embodiments, the second amino acid of the dipeptide is selected from the group consisting of A, C, D, E, F, G, H, I, K, L, M, N, P, Q, R, S, T, V, Y, and W.

In certain embodiments, the LINKER is/comprises GS.

In certain embodiments, the LINKER is/comprises SG.

In certain embodiments, the LINKER is/comprises GG.

In certain embodiments, the LINKER is/comprises SS.

In certain embodiments, the LINKER is/comprises HS.

In certain embodiments, the LINKER is a tripeptide. In certain embodiments, the first amino acid of the tripeptide is selected from the group consisting of A, C, D, E, F, G, H, I, K, L, M, N, P, Q, R, S, T, V, Y, and W. In certain embodiments, the second amino acid of the tripeptide is selected from the group consisting of A, C, D, E, F, G, H, I, K, L, M, N, P, Q, R, S, T, V, Y, and W. In certain embodiments, the third amino acid of the tripeptide is selected from the group consisting of A, C, D, E, F, G, H, I, K, L, M, N, P, Q, R, S, T, V, Y, and W.

In certain embodiments, the LINKER is/comprises GGG.

In certain embodiments, the LINKER is/comprises SGG.

In certain embodiments, the LINKER is/comprises GSG.

In certain embodiments, the LINKER is/comprises GGS.

In certain embodiments, the LINKER is/comprises GSS.

In certain embodiments, the LINKER is/comprises SGS.

In certain embodiments, the LINKER is/comprises SSG.

In certain embodiments, the LINKER is/comprises SSS.

In certain embodiments, the LINKER is/comprises GSC.

In certain embodiments, the LINKER is a tetrapeptide. In certain embodiments, the first amino acid of the tetrapeptide is selected from the group consisting of A, C, D, E, F, G, H, I, K, L, M, N, P, Q, R, S, T, V, Y, and W. In certain embodiments, the second amino acid of the tetrapeptide is selected from the group consisting of A, C, D, E, F, G, H, I, K, L, M, N, P, Q, R, S, T, V, Y, and W. In certain embodiments, the third amino acid of the tetrapeptide is selected from the group consisting of A, C, D, E, F, G, H, I, K, L, M, N, P, Q, R, S, T, V, Y, and W. In certain embodiments, the fourth amino acid of the tetrapeptide is selected from the group consisting of A, C, D, E, F, G, H, I, K, L, M, N, P, Q, R, S, T, V, Y, and W. In certain embodiments, the LINKER is/comprises GGGGSGGGGS (SEQ ID NO:5).

In certain embodiments, the LINKER is/comprises
(SEQ ID NO: 40)
SSTMVRS.
In certain embodiments, the LINKER is/comprises
(SEQ ID NO: 41)
SSTMVGS.
In certain embodiments, the LINKER is/comprises
(SEQ ID NO: 56)
SSTMVHS.

In certain embodiments, the LINKER is/comprises ELKTPLGDTTHTXPRZPAPELLGGP (SEQ ID NO:6), wherein each occurrence of X is C, G, or S, and wherein each occurrence of Z is C, G, or S. In certain non-limiting embodiments, at least one of X and Z is not C and formation of a disulfide bridge is prevented. In certain embodiments, SEQ ID NO:6 corresponds to the hinge region of Human IgG1.

### X1 and X2:

In certain embodiments, X1 is a covalent bond. In certain embodiments, X1 is the peptide of amino acid sequence RAFTNNRKSVSLKKRKKGNRS (SEQ ID NO:3) or a fragment thereof. In certain embodiments, X1 is the peptide of amino acid sequence RAFTNNRKSVSLKKRKKGNRSGGVKKPHRYRPGTVAL (SEQ ID NO:54). In certain embodiments, X1 is the peptide of amino acid sequence KQTARKAPRKQLATKAARKSAPATGGVKKPHRYRPGTVAL (SEQ ID NO:55).

In certain embodiments, X2 is a covalent bond. In certain embodiments, X2 is the peptide of amino acid sequence RAFTNNRKSVSLKKRKKGNRS (SEQ ID NO:3) or a fragment thereof. In certain embodiments, X2 is the peptide of amino acid sequence RAFTNNRKSVSLKKRKKGNRSGGVKKPHRYRPGTVAL (SEQ ID NO:54). In certain embodiments, X2 is the peptide of amino acid sequence KQTARKAPRKQLATKAARKSAPATGGVKKPHRYRPGTVAL (SEQ ID NO:55),

Without wishing to be limited by any theory, a DNAse1 and/or DNAse1L3 with a modified C-terminal tail relating to the highly charged N-terminal Histone H3 peptide has enhanced chromatin digestibility. Without wishing to be limited by any theory, the positively charged C-terminus tail of DNAse1L3 is exchangeable with the corresponding H3 peptide C-terminus peptide. Without wishing to be limited by any theory, adding sequences derived from the H3 peptide to the C-terminus of DNAse1 and/or DNAse1L3 increase the ability of these peptides to intercalate into the nucleosome, allowing for a more robust chromatin digestibility. See, for example, www dot biorxiv dot org/content/10 dot 1101/2021 dot 03 dot 15 dot 434949v3 dot full dot pdf.

### DNAse1:

An illustrative construct of the disclosure comprises the amino acid sequence of SEQ ID NO:7, wherein the bold sequence corresponds to the DNAse1 polypeptide, wherein the underlined sequence corresponds to the Fc, and wherein the italics sequence corresponds to the LINKER.

SEQ ID NO: 7
MRGMKLLGALLALAALLQGAVSLKIAAFNIQT
FGETKMSNATLVSYIVQILSRYDIALVQEVRD
SHLTAVGKLLDNLNQDAPDTYHYVVSEPLGRN
SYKERYLFVYRPDQVSAVDSYYYDDGCEPCGN
DTFNREPAIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLRGAVVPDSALPFN
FQAAYGLSDQLAQAISDHYPVEVMLKGSDKTH
TCPPCPAPELLGGPSVFLFPPKPKDTLMISRT
PEVTCVVVDVSHEDPEVKFNWYVDGVEVHNAK
TKPREEQYNSTYRVVSVLTVLHQDWLNGKEYK
CKVSNKALPAPIEKTISKAKGQPREPQVYTLP
PSREEMTKNQVSLTCLVKGFYPSDIAVEWESN
GQPENNYKTTPPVLDSDGSFFLYSKLTVDKSR
WQQGNVFSCSVMHEALHNHYTQKSLSLSPGK
SEQ ID NO: 46
MRGMKLLGALLALAALLQGAVSLKIAAFNIQT
FGETKMSNATLVSYIVQILSRYDIALVQEVRD
SHLTAVGKLLDNLNQDAPDTYHYVVSEPLGRN
SYKERYLFVYRPDQVSAVDSYYYDDGCEPCGN
DTFNREPAIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLRGAVVPDSALPFN
FQAAYGLSDQLAQAISDHYPVEVMLKGSCDKT
HTCPPCPAPELLGGPSVFLFPPKPKDTLMISR
TPEVTCVVVDVSHEDPEVKFNWYVDGVEVHNA
KTKPREEQYNSTYRVVSVLTVLHQDWLNGKEY
KCKVSNKALPAPIEKTISKAKGQPREPQVYTL
PPSREEMTKNQVSLTCLVKGFYPSDIAVEWES
NGQPENNYKTTPPVLDSDGSFFLYSKLTVDKS
RWQQGNVFSCSVMHEALHNHYTQKSLSLSPGK

In certain embodiments, the construct has one or more of the following mutations in Fc: C290S, C293S, M316Y, S318T, and/or T320E with respect to SEQ ID NO:7.

In certain embodiments, the construct has one or more of the following mutations in Fc: C285S, C291S, C294S, M317Y, S319T, and/or T321E with respect to SEQ ID NO:46.

An illustrative construct of the disclosure comprises the amino acid sequence of SEQ ID NO:8, wherein the bold sequence corresponds to the DNAse1 polypeptide, wherein the underlined sequence corresponds to the Fc, and wherein the italics sequence corresponds to the LINKER.

SEQ ID NO: 8
MRGMKLLGALLALAALLQGAVSLKIAAFNIQT
FGETKMSNATLVSYIVQILSRYDIALVQEVRD
SHLTAVGKLLDNLNQDAPDTYHYVVSEPLGRN
SYKERYLFVYRPDQVSAVDSYYYDDGCEPCGN
DTFNREPAIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLRGAVVPDSALPFN
FQAAYGLSDQLAQAISDHYPVEVMLKGSDKTH
TSPPSPAPELLGGPSVFLFPPKPKDTLYITRE
PEVTCVVVDVSHEDPEVKFNWYVDGVEVHNAK
TKPREEQYNSTYRVVSVLTVLHQDWLNGKEYK
CKVSNKALPAPIEKTISKAKGQPREPQVYTLP
PSREEMTKNQVSLTCLVKGFYPSDIAVEWESN
GQPENNYKTTPPVLDSDGSFFLYSKLTVDKSR
WQQGNVFSCSVMHEALHNHYTQKSLSLSPGK

An illustrative construct of the disclosure comprises the amino acid sequence of SEQ ID NO:47, wherein the bold sequence corresponds to the DNAse1 polypeptide, wherein the underlined sequence corresponds to the Fc, and wherein the italics sequence corresponds to the LINKER.

SEQ ID NO: 47
MRGMKLLGALLALAALLQGAVSLKIAAFNIQT
FGETKMSNATLVSYIVQILSRYDIALVQEVRD
SHLTAVGKLLDNLNQDAPDTYHYVVSEPLGRN
SYKERYLFVYRPDQVSAVDSYYYDDGCEPCGN
DTFNREPAIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLRGAVVPDSALPFN
FQAAYGLSDQLAQAISDHYPVEVMLKGSSDKT
HISPPSPAPELLGGPSVFLFPPKPKDTLYITR
EPEVTCVVVDVSHEDPEVKFNWYVDGVEVHNA
KTKPREEQYNSTYRVVSVLTVLHQDWLNGKEY
KCKVSNKALPAPIEKTISKAKGQPREPQVYTL
PPSREEMTKNQVSLTCLVKGFYPSDIAVEWES
NGQPENNYKTTPPVLDSDGSFFLYSKLTVDKS
RWQQGNVFSCSVMHEALHNHYTQKSLSLSPGK

In certain embodiments, the construct lacks at least a portion of the signal peptide of DNAse1 corresponding to residues 1-22 of SEQ ID NO:1. In certain embodiments, the construct lacks the signal peptide of DNAse1 corresponding to residues 1-22 of SEQ ID NO:1.

A non-limiting list of contemplated mutations in the DNAse1 domain of the constructs of the disclosure with respect to SEQ ID NO:1 include but are not limited to Q31R, E35R, Y46H, Y46S, V88N, N96K, D109N, V111T, A136F, R148S, E149N, M186I, L208P, D220N, R244Q, D250N, A252T, G262N, D265N, and L267T.

In certain embodiments, the DNAse1 domain of the construct comprises the mutation Q31R with respect to SEQ ID NO:1. In certain embodiments, the DNAse1 domain of the construct comprises the mutation E35R with respect to SEQ ID NO:1. In certain embodiments, the DNAse1 domain of the construct comprises the mutation Y46H with respect to SEQ ID NO:1. In certain embodiments, the DNAse1 domain of the construct comprises the mutation Y46S with respect to SEQ ID NO:1. In certain embodiments, the DNAse1 domain of the construct comprises the mutation V88N with respect to SEQ ID NO:1. In certain embodiments, the DNAse1 domain of the construct comprises the mutation N96K with respect to SEQ ID NO:1. In certain embodiments, the DNAse1 domain of the construct comprises the mutation D109N with respect to SEQ ID NO:1. In certain embodiments, the DNAse1 domain of the construct comprises the mutation V111T with respect to SEQ ID NO:1. In certain embodiments, the DNAse1 domain of the construct comprises the mutation A136F with respect to SEQ ID NO:1. In certain embodiments, the DNAse1 domain of the construct comprises the mutation R148S with respect to SEQ ID NO:1. In certain embodiments, the DNAse1 domain of the construct comprises the mutation E149N with respect to SEQ ID NO:1. In certain embodiments, the DNAse1 domain of the construct comprises the mutation M186I with respect to SEQ ID NO:1. In certain embodiments, the DNAse1 domain of the construct comprises the mutation L208P with respect to SEQ ID NO:1. In certain embodiments, the DNAse1 domain of the construct comprises the mutation D220N with respect to SEQ ID NO:1. In certain embodiments, the DNAse1 domain of the construct comprises the mutation R244Q with respect to SEQ ID NO:1. In certain embodiments, the DNAse1 domain of the construct comprises the mutation D250N with respect to SEQ ID NO:1. In certain embodiments, the DNAse1 domain of the construct comprises the mutation A252T with respect to SEQ ID NO:1. In certain embodiments, the DNAse1 domain of the construct comprises the mutation G262N with respect to SEQ ID NO:1. In certain embodiments, the DNAse1 domain of the construct comprises the mutation D265N with respect to SEQ ID NO:1. In certain embodiments, the DNAse1 domain of the construct comprises the mutation L267T with respect to SEQ ID NO:1.

In certain embodiments, the human DNAse1 comprises the mutations Q31R, N96K, A136F, and R244Q with respect to SEQ ID NO:1.

In certain embodiments, the human DNAse1 comprises residues 1-282 of SEQ ID NO:7.

In certain embodiments, the human DNAse1 comprises residues 1-282 of SEQ ID NO:46.

In certain embodiments, the human DNAse1 comprises residues 1-282 of SEQ ID NO:8.

In certain embodiments, the human DNAse1 comprises residues 1-282 of SEQ ID NO:47.

In certain embodiments, the human DNAse1 comprises residues 1-282 of SEQ ID NO:9.

In certain embodiments, the human DNAse1 comprises residues 1-282 of SEQ ID NO:48.

In certain embodiments, the human DNAse1 comprises residues 1-282 of SEQ ID NO:10.

In certain embodiments, the human DNAse1 comprises residues 1-282 of SEQ ID NO:49.

In certain embodiments, the human DNAse1 comprises residues 1-282 of SEQ ID NO:11.

In certain embodiments, the human DNAse1 comprises residues 1-282 of SEQ ID NO:50.

In certain embodiments, the human DNAse1 comprises residues 1-282 of SEQ ID NO:12.

In certain embodiments, the human DNAse1 comprises residues 1-282 of SEQ ID NO:51.

In certain embodiments, the human DNAse1 comprises residues 1-282 of SEQ ID NO:13.

In certain embodiments, the human DNAse1 comprises residues 1-282 of SEQ ID NO:52.

In certain embodiments, the human DNAse1 comprises residues 1-282 of SEQ ID NO:14.

In certain embodiments, the human DNAse1 comprises residues 1-282 of SEQ ID NO:15.

In certain embodiments, the human DNAse1 comprises residues 1-282 of SEQ ID NO:53.

In certain embodiments, the human DNAse1 comprises residues 1-282 of SEQ ID NO:16.

In certain embodiments, the human DNAse1 comprises residues 1-282 of SEQ ID NO:17.

In certain embodiments, the human DNAse1 comprises residues 1-282 of SEQ ID NO:44.

In certain embodiments, the mouse DNAse1 comprises residues 1-284 of SEQ ID NO:32.

In certain embodiments, the mouse DNAse1 comprises residues 1-284 of SEQ ID NO:33.

In certain embodiments, the mouse DNAse1 comprises residues 1-284 of SEQ ID NO:34.

In certain embodiments, the mouse DNAse1 comprises residues 1-284 of SEQ ID NO:35.

In certain non-limiting embodiments, the mutation A136F with respect to SEQ ID NO:1 decreases actin binding of the construct.

In certain non-limiting embodiments, the mutation(s) E35R, Y46H, Y46S, R148S, E149N, M186I, L208P, and/or D220N increase the enzymatic activity and/or potency of the construct.

In certain non-limiting embodiments, the mutation(s) V88N, D109N, V111T, G262N, D265N, and/or L267T modify the overall glycosylation status of the construct.

Non-limiting examples of constructs of the disclosure comprise the following amino acid sequences, wherein the bold sequence corresponds to the DNAse1 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 9
MRGMKLLGALLALAALLQGAVSLKIAAFNIQTFGRTKMSNATLVSYIVQILSRYDIALVQEVRD
SHLTAVGKLLDNLNQDAPDTYHYNVSEPLGRNSYKERYLFVYRPNQTSAVDSYYYDDGCEPCGN
DTFNREPFIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYNLSNQTAQAISDHYPVEVMLKGSDKTHTSPPSPAPELLGGPSVFLFPPKPKDTLYITRE
PEVTCVVVDVSHEDPEVKENWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGK
SEQ ID NO: 48
MRGMKLLGALLALAALLQGAVSLKIAAFNIQTFGRTKMSNATLVSYIVQILSRYDIALVQEVRD
SHLTAVGKLLDNLNQDAPDTYHYNVSEPLGRNSYKERYLFVYRPNQTSAVDSYYYDDGCEPCGN
DTFNREPFIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYNLSNQTAQAISDHYPVEVMLKGSSDKTHTSPPSPAPELLGGPSVFLFPPKPKDTLYITR
EPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
SEQ ID NO: 10
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKRAFTNNRKSVSLKKRKKGNRSGSDKTHTSPPSPAPELL
GGPSVFLFPPKPKDTLYITREPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSV
MHEALHNHYTQKSLSLSPGKRAFTNNRKSVSLKKRKKGNRS
SEQ ID NO: 49
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKRAFTNNRKSVSLKKRKKGNRSGSSDKTHTSPPSPAPEL
LGGPSVFLFPPKPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCS
VMHEALHNHYTQKSLSLSPGKRAFTNNRKSVSLKKRKKGNRS
SEQ ID NO: 11
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKGSDKTHTSPPSPAPELLGGPSVFLFPPKPKDTLYITRE
PEVTCVVVDVSHEDPEVKENWYVDGVEVHNAKTKPREEQYNSTYRVVSVLTVLHQDWLNGKEYK
CKVSNKALPAPIEKTISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHNHYTQKSLSLSPGKR
AFTNNRKSVSLKKRKKGNRS
SEQ ID NO: 50
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKGSSDKTHTSPPSPAPELLGGPSVFLFPPKPKDTLYITR
EPEVTCVVVDVSHEDPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK
RAFTNNRKSVSLKKRKKGNRS
SEQ ID NO: 12
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKRAFTNNRKSVSLKKRKKGNRSGSDKTHTSPPSPAPELL
GGPSVFLFPPKPKDTLYITREPEVTCVVVDVSHEDPEVKENWYVDGVEVHNAKTKPREEQYNST
YRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 51
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKRAFTNNRKSVSLKKRKKGNRSGSSDKTHTSPPSPAPEL
LGGPSVFLFPPKPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 13
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKGGGGSGGGGSDKTHTSPPSPAPELLGGPSVFLFPPKPK
DTLYITREPEVTCVVVDVSHEDPEVKENWYVDGVEVHNAKTKPREEQYNSTYRVVSVLTVLHQD
WLNGKEYKCKVSNKALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCSVMHEALHNHYTQKS
LSLSPGK
SEQ ID NO: 52
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKGGGGSGGGGSSDKTHTSPPSPAPELLGGPSVELFPPKP
KDTLYITREPEVTCVVVDVSHEDPEVKFNWYVDGVEVHNAKTKPREEQYNSTYRVVSVLTVLHQ
DWLNGKEYKCKVSNKALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSCSVMHEALHNHYTQK
SLSLSPGK
SEQ ID NO: 14
MRGMKLLGALLALAALLQGAVSLKIAAFNIQTFGETKMSNATLVSYIVQILSRYDIALVQEVRD
SHLTAVGKLLDNLNQDAPDTYHYVVSEPLGRNSYKERYLFVYRPDQVSAVDSYYYDDGCEPCGN
DTFNREPAIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLRGAVVPDSALPFN
FQAAYGLSDQLAQAISDHYPVEVMLKELKTPLGDTTHTXPRZPAPELLGGPDKTHTSPPSPAPE
LLGGPSVFLFPPKPKDTLYITREPEVTCVVVDVSHEDPEVKENWYVDGVEVHNAKTKPREEQYN
STYRVVSVLTVLHQDWLNGKEYKCKVSNKALPAPIEKTISKAKGQPREPQVYTLPPSREEMTKN
QVSLTCLVKGFYPSDIAVEWESNGQPENNYKTTPPVLDSDGSFFLYSKLTVDKSRWQQGNVFSC
SVMHEALHNHYTQKSLSLSPGK

wherein X and Z are independently Cys, Gly, or Ser.

In certain non-limiting embodiments, wherein at least one of X and Z is not Cys (C) formation of disulfide bridge is prevented.

SEQ ID NO: 15
MRGMKLLGALLALAALLQGAVSLKIAAFNIRT
FGETKMSNATLVSYIVQILSRYDIALVQEVRD
SHLTAVGKLLDNLNQDAPDTYHYVVSEPLGRK
SYKERYLFVYRPDQVSAVDSYYYDDGCEPCGN
DTFNREPFIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLQGAVVPDSALPFN
FQAAYGLSDQLAQAISDHYPVEVMLKGSCDKT
HTCPPCPAPELLGGPSVFLFPPKPKDTLYITR
EPEVTCVVVDVSHEDPEVKFNWYVDGVEVHNA
KTKPREEQYNSTYRVVSVLTVLHQDWLNGKEY
KCKVSNKALPAPIEKTISKAKGQPREPQVYTL
PPSREEMTKNQVSLTCLVKGFYPSDIAVEWES
NGQPENNYKTTPPVLDSDGSFFLYSKLTVDKS
RWQQGNVFSCSVMHEALHNHYTQKSLSLSPGK
SEQ ID NO: 53
MRGMKLLGALLALAALLQGAVSLKIAAFNIRT
FGETKMSNATLVSYIVQILSRYDIALVQEVRD
SHLTAVGKLLDNLNQDAPDTYHYVVSEPLGRK
SYKERYLFVYRPDQVSAVDSYYYDDGCEPCGN
DTFNREPFIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLQGAVVPDSALPFN
FQAAYGLSDQLAQAISDHYPVEVMLKGSCSDK
THTCPPCPAPELLGGPSVELFPPKPKDTLYIT
REPEVTCVVVDVSHEDPEVKFNWYVDGVEVHN
AKTKPREEQYNSTYRVVSVLTVLHQDWLNGKE
YKCKVSNKALPAPIEKTISKAKGQPREPQVYT
LPPSREEMTKNQVSLTCLVKGFYPSDIAVEWE
SNGQPENNYKTTPPVLDSDGSFFLYSKLTVDK
SRWQQGNVFSCSVMHEALHNHYTQKSLSLSPG
K
SEQ ID NO: 16
MRGMKLLGALLALAALLQGAVSLKIAAFNIRT
FGETKMSNATLVSYIVQILSRYDIALVQEVRD
SHLTAVGKLLDNLNQDAPDTYHYVVSEPLGRK
SYKERYLFVYRPDQVSAVDSYYYDDGCEPCGN
DTFNREPFIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLQGAVVPDSALPFN
FQAAYGLSNQTAQAISDHYPVEVMLKGSCDKT
HTCPPCPAPELLGGPSVFLFPPKPKDTLYITR
EPEVTCVVVDVSHEDPEVKFNWYVDGVEVHNA
KTKPREEQYNSTYRVVSVLTVLHQDWLNGKEY
KCKVSNKALPAPIEKTISKAKGQPREPQVYTL
PPSREEMTKNQVSLTCLVKGFYPSDIAVEWES
NGQPENNYKTTPPVLDSDGSFFLYSKLTVDKS
RWQQGNVFSCSVMHEALHNHYTQKSLSLSPGK
SEQ ID NO: 17
MRGMKLLGALLALAALLQGAVSLKIAAFNIRT
FGETKMSNATLVSYIVQILSRYDIALVQEVRD
SHLTAVGKLLDNLNQDAPDTYHYVVSEPLGRK
SYKERYLFVYRPDQVSAVDSYYYDDGCEPCGN
DTFNREPFIVRFFSRFTEVREFAIVPLHAAPG
DAVAEIDALYDVYLDVQEKWGLEDVMLMGDFN
AGCSYVRPSQWSSIRLWTSPTFQWLIPDSADT
TATPTHCAYDRIVVAGMLLQGAVVPNSTLPFN
FQAAYGLSDQLAQAISDHYPVEVMLKGSCDKT
HTCPPCPAPELLGGPSVFLFPPKPKDTLYITR
EPEVTCVVVDVSHEDPEVKENWYVDGVEVHNA
KTKPREEQYNSTYRVVSVLTVLHQDWLNGKEY
KCKVSNKALPAPIEKTISKAKGQPREPQVYTL
PPSREEMTKNQVSLTCLVKGFYPSDIAVEWES
NGQPENNYKTTPPVLDSDGSFFLYSKLTVDKS
RWQQGNVFSCSVMHEALHNHYTQKSLSLSPGK

### DNAse1L3:

An illustrative construct of the disclosure comprises the amino acid sequence of SEQ ID NO:18, wherein the bold sequence corresponds to the DNAse1L3 polypeptide, wherein the underlined sequence corresponds to the Fc, and wherein the italics sequence corresponds to the LINKER.

SEQ ID NO: 18
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSGSDKTHTCPPCPAPE
LLGGPSVFLFPPKPKDTLMISRTPEVTCVVVD
VSHEDPEVKFNWYVDGVEVHNAKTKPREEQYN
STYRVVSVLTVLHQDWLNGKEYKCKVSNKALP
APIEKTISKAKGQPREPQVYTLPPSREEMTKN
QVSLTCLVKGFYPSDIAVEWESNGQPENNYKT
TPPVLDSDGSFFLYSKLTVDKSRWQQGNVFSC
SVMHEALHNHYTQKSLSLSPGK

In certain embodiments, the construct has one or more of the following mutations in Fc: C313S, C316S, M339Y, S341T, and/or T342E with respect to SEQ ID NO:18.

An illustrative construct of the disclosure comprises the amino acid sequence of SEQ ID NO:19, wherein the bold sequence corresponds to the DNAse1L3 polypeptide, wherein the underlined sequence corresponds to the Fc, and wherein the italics sequence corresponds to the LINKER.

SEQ ID NO: 19
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSGSDKTHTSPPSPAPE
LLGGPSVFLFPPKPKDTLYITREPEVTCVVVD
VSHEDPEVKENWYVDGVEVHNAKTKPREEQYN
STYRVVSVLTVLHQDWLNGKEYKCKVSNKALP
APIEKTISKAKGQPREPQVYTLPPSREEMTKN
QVSLTCLVKGFYPSDIAVEWESNGQPENNYKT
TPPVLDSDGSFFLYSKLTVDKSRWQQGNVFSC
SVMHEALHNHYTQKSLSLSPGK

In certain embodiments, the construct lacks at least a portion of the signal peptide of DNAse1L3 corresponding to residues 1-20 of SEQ ID NO:2. In certain embodiments, the construct lacks the signal peptide of DNAse1L3 corresponding to residues 1-20 of SEQ ID NO:2.

In certain embodiments, the construct lacks at least a portion of the nuclear localization sequence (NLS) of the DNAse1L3 polypeptide. In certain embodiments, the construct lacks residues 291-305 of SEQ ID NO:2. In certain embodiments, the construct lacks residues 292-304 of SEQ ID NO:2. In certain embodiments, the construct lacks residues 296-304 of SEQ ID NO:2. In certain embodiments, the construct lacks residues A-B of SEQ ID NO:2, wherein A ranges from 291 to 296 and B ranges from 304 to 305.

A non-limiting list of contemplated mutations in the Fc domain of the constructs of the disclosure, with respect to SEQ ID NO:18, include C313S, C316S, M339Y, S341T, and/or T342E.

A non-limiting list of contemplated mutations in the DNAse1L3 domain of the constructs of the disclosure, with respect to SEQ ID NO:2, include E33R, M42T, V44H, V88T, N96K, A127N, V129T, K147S, D148N, L207P, D219N, and/or V254T.

In certain embodiments, the DNAseIL3 domain of the construct comprises the mutation E33R with respect to SEQ ID NO:2. In certain embodiments, the DNAseIL3 domain of the construct comprises the mutation M42T with respect to SEQ ID NO:2. In certain embodiments, the DNAseIL3 domain of the construct comprises the mutation V44H with respect to SEQ ID NO:2. In certain embodiments, the DNAseIL3 domain of the construct comprises the mutation V88T with respect to SEQ ID NO:2. In certain embodiments, the DNAseIL3 domain of the construct comprises the mutation N96K with respect to SEQ ID NO:2. In certain embodiments, the DNAseIL3 domain of the construct comprises the mutation A127N with respect to SEQ ID NO:2. In certain embodiments, the DNAseIL3 domain of the construct comprises the mutation V129T with respect to SEQ ID NO:2. In certain embodiments, the DNAseIL3 domain of the construct comprises the mutation K147S with respect to SEQ ID NO:2. In certain embodiments, the DNAseIL3 domain of the construct comprises the mutation D148N with respect to SEQ ID NO:2. In certain embodiments, the DNAseIL3 domain of the construct comprises the mutation L207P with respect to SEQ ID NO:2. In certain embodiments, the DNAseIL3 domain of the construct comprises the mutation D219N with respect to SEQ ID NO:2. In certain embodiments, the DNAseIL3 domain of the construct comprises the mutation V254T with respect to SEQ ID NO:2.

In certain embodiments, the human DNAse1L3 comprises residues 1-305 of SEQ ID NO:18.

In certain embodiments, the human DNAse1L3 comprises residues 1-305 of SEQ ID NO:19.

In certain embodiments, the human DNAse1L3 comprises residues 1-305 of SEQ ID NO:20.

In certain embodiments, the human DNAse1L3 comprises residues 1-305 of SEQ ID NO:21.

In certain embodiments, the human DNAse1L3 comprises residues 1-305 of SEQ ID NO:22.

In certain embodiments, the human DNAse1L3 comprises residues 1-305 of SEQ ID NO:23.

In certain embodiments, the human DNAse1L3 comprises residues 1-305 of SEQ ID NO:24.

In certain embodiments, the human DNAse1L3 comprises residues 1-290 of SEQ ID NO:25.

In certain embodiments, the human DNAse1L3 comprises residues 1-305 of SEQ ID NO:26.

In certain embodiments, the human DNAse1L3 comprises residues 1-305 of SEQ ID NO:27.

In certain embodiments, the human DNAse1L3 comprises residues 1-305 of SEQ ID NO:28.

In certain embodiments, the mouse DNAse1L3 comprises residues 1-310 of SEQ ID NO:36.

In certain embodiments, the mouse DNAse1L3 comprises residues 1-297 of SEQ ID NO:37.

In certain embodiments, the mouse DNAse1L3 comprises residues 1-310 of SEQ ID NO:38.

In certain embodiments, the mouse DNAse1L3 comprises residues 1-310 of SEQ ID NO:39.

In certain non-limiting embodiments, the mutation A136F with respect to SEQ ID NO:1 decreases actin binding of the construct.

In certain non-limiting embodiments, the mutation(s) E33R, V44H, N96K, K147S, D148N, L207P, and/or D219N with respect to SEQ ID NO:2 increase(s) the enzymatic activity and/or potency of the construct.

In certain non-limiting embodiments, the mutation V254T modifies the overall glycosylation status of the construct.

Non-limiting examples of constructs of the disclosure comprise the following amino acid sequences, wherein the bold sequence corresponds to the DNAse1L3 polypeptide, wherein the underlined sequence corresponds to the Fc, wherein the italics sequence corresponds to the LINKER, and wherein the italics/underlined sequence corresponds to X1/X2. Certain mutations are shown as doubly underlined.

SEQ ID NO: 20
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSGSDKTHTSPPSPAPE
LLGGPSVFLFPPKPKDTLYITREPEVTCVVVD
VSHEDPEVKENWYVDGVEVHNAKTKPREEQYN
STYRVVSVLTVLHQDWLNGKEYKCKVSNKALP
APIEKTISKAKGQPREPQVYTLPPSREEMTKN
QVSLTCLVKGFYPSDIAVEWESNGQPENNYKT
TPPVLDSDGSFFLYSKLTVDKSRWQQGNVFSC
SVMHEALHNHYTQKSLSLSPGKRAFTNNRKSV
SLKKRKKGNRS
SEQ ID NO: 21
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVED
FQKAYKLTEEEALDVSDHFPVEFKLQSSRAFT
NSKKSVTLRKKTKSKRSRAFTNNRKSVSLKKR
KKGNRSGSDKTHTSPPSPAPELLGGPSVFLFP
PKPKDTLYITREPEVTCVVVDVSHEDPEVKFN
WYVDGVEVHNAKTKPREEQYNSTYRVVSVLTV
LHQDWLNGKEYKCKVSNKALPAPIEKTISKAK
GQPREPQVYTLPPSREEMTKNQVSLTCLVKGF
YPSDIAVEWESNGQPENNYKTTPPVLDSDGSF
FLYSKLTVDKSRWQQGNVFSCSVMHEALHNHY
TQKSLSLSPGKRAFTNNRKSVSLKKRKKGNRS
SEQ ID NO: 22
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSRAFTNNRKSVSLKKR
KKGNRSGSDKTHTSPPSPAPELLGGPSVFLFP
PKPKDTLYITREPEVTCVVVDVSHEDPEVKFN
WYVDGVEVHNAKTKPREEQYNSTYRVVSVLTV
LHQDWLNGKEYKCKVSNKALPAPIEKTISKAK
GQPREPQVYTLPPSREEMTKNQVSLTCLVKGF
YPSDIAVEWESNGQPENNYKTTPPVLDSDGSF
FLYSKLTVDKSRWQQGNVFSCSVMHEALHNHY
TQKSLSLSPGK
SEQ ID NO: 23
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSGGGGSGGGGSDKTHT
SPPSPAPELLGGPSVFLFPPKPKDTLYITREP
EVTCVVVDVSHEDPEVKENWYVDGVEVHNAKT
KPREEQYNSTYRVVSVLTVLHQDWLNGKEYKC
KVSNKALPAPIEKTISKAKGQPREPQVYTLPP
SREEMTKNQVSLTCLVKGFYPSDIAVEWESNG
QPENNYKTTPPVLDSDGSFFLYSKLTVDKSRW
QQGNVFSCSVMHEALHNHYTQKSLSLSPGK
SEQ ID NO: 24
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSKKSVTLRKKTKSKRSELKTPLGDTTHTXPR
ZPAPEFLGGPDKTHTXPPZPAPELLGGPSVFL
FPPKPKDTLYITREPEVTCVVVDVSHEDPEVK
FNWYVDGVEVHNAKTKPREEQYNSTYRVVSVL
TVLHQDWLNGKEYKCKVSNKALPAPIEKTISK
AKGQPREPQVYTLPPSREEMTKNQVSLTCLVK
GFYPSDIAVEWESNGQPENNYKTTPPVLDSDG
SFFLYSKLTVDKSRWQQGNVFSCSVMHEALHN
HYTQKSLSLSPGK*

wherein each occurrence of X and Z is independently Cys, Gly, or Ser.

In certain non-limiting embodiments, wherein at least one of X and Z is not Cys (C) formation of disulfide bridge is prevented.

SEQ ID NO: 25
MSRELAPLLLLLLSIHSALAMRICSFNVRSFG
ESKQEDKNAMDVIVKVIKRCDIILVMEIKDSN
NRICPILMEKLNRNSRRGITYNYVISSRLGRK
TYKEQYAFLYKEKLVSVKRSYHYHDYQDGDAD
VFSREPFVVWFQSPHTAVKDFVIIPLHTTPET
SVKEIDELVEVYTDVKHRWKAENFIFMGDFNA
GCSYVPKKAWKNIRLRTDPRFVWLIGDQEDTT
VKKSTNCAYDRIVLRGQEIVSSVVPKSNSVFD
FQKAYKLTEEEALDVSDHFPVEFKLQSSRAFT
NSGSDKTHTXPPZPAPELLGGPSVFLFPPKPK
DTLYITREPEVTCVVVDVSHEDPEVKFNWYVD
GVEVHNAKTKPREEQYNSTYRVVSVLTVLHQD
WLNGKEYKCKVSNKALPAPIEKTISKAKGQPR
EPQVYTLPPSREEMTKNQVSLTCLVKGFYPSD
IAVEWESNGQPENNYKTTPPVLDSDGSFFLYS
KLTVDKSRWQQGNVFSCSVMHEALHNHYTQKS
LSLSPGK

wherein X and Z are independently C, G, or S.

In certain non-limiting embodiments, wherein at least one of X and Z is not Cys (C) formation of disulfide bridge is prevented.

SEQ ID NO: 26
MSRELAPLLLLLLSIHSALAMRICSFNVRSFGRSKQEDKNATDVIVKVIKRCDIILVMEIKDSN
NRICPILMEKLNRNSRRGITYNYVISSRLGRKTYKEQYAFLYKEKLVSVKRSYHYHDYQDGDAD
VFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDTLYITREPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFS
CSVMHEALHNHYTQKSLSLSPGK*
SEQ ID NO: 27
MSRELAPLLLLLLSIHSALAMRICSFNVRSFGRSKQEDKNATDVIVKVIKRCDIILVMEIKDSN
NRICPILMEKLNRNSRRGITYNYVISSRLGRKTYKEQYAFLYKEKLVSVKRSYHYHDYQDGDND
TFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDTLYITREPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFS
CSVMHEALHNHYTQKSLSLSPGK
SEQ ID NO: 28
MSRELAPLLLLLLSIHSALAMRICSFNVRSFGRSKQEDKNATDVIVKVIKRCDIILVMEIKDSN
NRICPILMEKLNRNSRRGITYNYTISSRLGRKTYKEQYAFLYKEKLVSVKRSYHYHDYQDGDAD
VFSREPFVVWFQSPHTAVKDFVIIPLHTTPETSVKEIDELVEVYTDVKHRWKAENFIFMGDFNA
GCSYVPKKAWKNIRLRTDPRFVWLIGDQEDTTVKKSTNCAYDRIVLRGQEIVSSVVPKSNSVFD
FQKAYKLTEEEALDVSDHFPVEFKLQSSRAFTNSKKSVTLRKKTKSKRSGSCDKTHTCPPCPAP
ELLGGPSVFLFPPKPKDTLYITREPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFS
CSVMHEALHNHYTQKSLSLSPGK
(Sequence 1171 - mouse DNAse1 construct)
SEQ ID NO: 32
MRYTGLMGTLLTLVNLLQLAGTLRIAAFNIRTFGETKMSNATLSVYFVKILSRYDIAVIQEVRD
SHLVAVGKLLDELNRDKPDTYRYVVSEPLGRKSYKEQYLFVYRPDQVSILDSYQYDDGCEPCGN
DTFSREPAIVKFFSPYTEVQEFAIVPLHAAPTEAVSEIDALYDVYLDVWQKWGLEDIMFMGDFN
AGCSYVTSSQWSSIRLRTSPIFQWLIPDSADTTVTSTHCAYDRIVVAGALLQAAVVPNSAVPFD
FQAEYGLSNQLAEAISDHYPVEVTLRKISSTMVRSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1671 - mouse DNAse1 construct)
SEQ ID NO: 33
MRYTGLMGTLLTLVNLLQLAGTLRIAAFNIRTFGETKMSNATLSVYFVKILSRYDIAVIQEVRD
SHLVAVGKLLDELNRDKPDTYRYVVSEPLGRKSYKEQYLFVYRPDQVSILDSYQYDDGCEPCGN
DTFSREPFIVKFFSPYTEVQEFAIVPLHAAPTEAVSEIDALYDVYLDVWQKWGLEDIMFMGDFN
AGCSYVTSSQWSSIRLRTSPIFQWLIPDSADTTVTSTHCAYDRIVVAGALLQAAVVPNSAVPFD
FQAEYGLSNQLAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1687 - mouse DNAse1 construct)
SEQ ID NO: 34
MRYTGLMGTLLTLVNLLQLAGTLRIAAFNIRTFGRTKMSNATLSVYFVKILSRYDIAVIQEVRD
SHLVAVGKLLDELNRDKPDTYRYNVSEPLGRKSYKEQYLFVYRPDQVSILDSYQYDDGCEPCGN
DTFSREPAIVKFFSPYTEVQEFAIVPLHAAPTEAVSEIDALYDVYLDVWQKWGLEDIMFMGDFN
AGCSYVTSSQWSSIRLRTSPIFQWLIPDSADTTVTSTHCAYDRIVVAGALLQAAVVPNSAVPFD
FQAEYNLSNQLAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1687b - mouse DNAse1 construct)
SEQ ID NO: 74
MRYTGLMGTLLTLVNLLQLAGTLRIAAFNIRTFGRTKMSNATLSVYFVKILSRYDIAVIQEVRDSH
LVAVGKLLDELNRDKPDTYRYNVSEPLGRKSYKEQYLFVYRPDQVSILDSYQYDDGCEPCGNDT
FSREPAIVKFFSPYTEVQEFAIVPLHAAPTEAVSEIDALYDVYLDVWQKWGLEDIMFMGDFNAG
CSYVTSSQWSSIRLRTSPIFQWLIPDSADTTVTSTHCAYDRIVVAGALLQAAVVPNSAVPFDFQ
AEYGLSNQLAEAISDHYPVEVTLRKISSTMVGSGCKPCICTVPEVSSVFIFPPKPKDVLYITLEP
KVTCVVVDISKDDPEVQFSWFVDDVEVHTAQTQPREEQFNSTERSVSELPIMHQDWINGKEFKC
RVNSAAFPAPIEKTISKTKGRPKAPQVYTIPPPKEQMAKDKVSLTCMITDFFPEDITVEWQWNG
QPAENYKNTQPIMDTDGSYFVYSKLNVQKSNWEAGNTFTCSVLHEGLHNHHTEKSLSHSPGK
(Sequence 1689 - mouse DNAse1 construct)
SEQ ID NO: 35
MRYTGLMGTLLTLVNLLQLAGTLRIAAFNIRTFGRTKMSNATLSVYFVKILSRYDIAVIQEVRD
SHLVAVGKLLDELNRDKPDTYRYNVSEPLGRKSYKEQYLFVYRPDQVSILDSYQYDDGCEPCGN
DTFSREPAIVKFFSPYTEVQEFAIVPLHAAPTEAVSEIDALYDVYLDVWQKWGLEDIMFMGDFN
AGCSYVTSSQWSSIRLRTSPIFQWLIPDSADTTVTSTHCAYDRIVVAGALLQAAVVPNSAVPFD
FQAEYGLSNQTAEAISDHYPVEVTLRKISSTMVGSGCKPCICTVPEVSSVFIFPPKPKDVLYIT
LEPKVTCVVVDISKDDPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTCMITDFFPEDITVEWQ
WNGQPAENYKNTQPIMDTDGSYFVYSKLNVQKSNWEAGNTFTCSVLHEGLHNHHTEKSLSHSPG
K
(Sequence 1584 - mouse DNAse1L3 construct)
SEQ ID NO: 36
MSLHPASPRLASLLLFILALHDTLALRLCSFNVRSFGASKKENHEAMDIIVKIIKRCDLILLME
IKDSSNNICPMLMEKLNGNSRRSTTYNYVISSRLGRNTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSLSHSPGK
(Sequence 1596 - mouse DNAse1L3 construct)
SEQ ID NO: 37
MSLHPASPRLASLLLFILALHDTLALRLCSFNVRSFGASKKENHEAMDIIVKIIKRCDLILLME
IKDSSNNICPMLMEKLNGNSRRSTTYNYVISSRLGRNTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRSGCKPCICTVPEVSSVFIFPPKPK
DVLYITLEPKVTCVVVDISKDDPEVQFSWFVDDVEVHTAQTQPREEQFNSTFRSVSELPIMHQD
WLNGKEFKCRVNSAAFPAPIEKTISKTKGRPKAPQVYTIPPPKEQMAKDKVSLTCMITDFFPED
ITVEWQWNGQPAENYKNTQPIMDTDGSYFVYSKLNVQKSNWEAGNTFTCSVLHEGLHNHHTEKS
LSHSPGK
(Sequence 1615 - mouse DNAse1L3 construct)
SEQ ID NO: 38
MSLHPASPRLASLLLFILALHDTLALRLCSFNVRSFGRSKKENHEAMDIIVKIIKRCDLILLME
IKDSSNNICPMLMEKLNGNSRRSTTYNYVISSRLGRKTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
SGVFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSLSHSPGK
(Sequence 1669 - mouse DNAse1L3 construct)
SEQ ID NO: 39
MSLHPASPRLASLLLFILALHDTLALRLCSFNVRSFGRSKKENHEAMDIIVKIIKRCDLILLME
IKDSSNNICPMLMEKLNGNSRRSTTYNYVISSRLGRKTYKEQYAFVYKEKLVSVKTKYHYHDYQ
DGDTDVFSREPFVVWFHSPFTAVKDFVIVPLHTTPETSVKEIDELVDVYTDVRSQWKTENFIFM
GDFNAGCSYVPKKAWQNIRLRTDPKFVWLIGDQEDTTVKKSTSCAYDRIVLCGQEIVNSVVPRS
NGTFDFQKAYDLSEEEALDVSDHFPVEFKLQSSRAFTNNRKSVSLKKRKKGNRSSSTMVGSGCK
PCICTVPEVSSVFIFPPKPKDVLYITLEPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKE
QMAKDKVSLTCMITDFFPEDITVEWQWNGQPAENYKNTQPIMDTDGSYFVYSKLNVQKSNWEAG
NTFTCSVLHEGLHNHHTEKSLSHSPGK
SEQ ID NO: 54
RAFTNNRKSVSLKKRKKGNRSGGVKKPHRYRPGTVAL
SEQ ID NO: 55
KQTARKAPRKQLATKAARKSAPATGGVKKPHRYRPGTVAL
(Sequence 1833 - human DNAse1 construct)
SEQ ID NO: 44
MRGMKLLGALLALAALLQGAVSLKIAAFNIRTFGETKMSNATLVSYIVQILSRYDIALVQEVRD
SHLTAVGKLLDNLNQDAPDTYHYVVSEPLGRKSYKERYLFVYRPDQVSAVDSYYYDDGCEPCGN
DTFNREPFIVRFFSRFTEVREFAIVPLHAAPGDAVAEIDALYDVYLDVQEKWGLEDVMLMGDFN
AGCSYVRPSQWSSIRLWTSPTFQWLIPDSADTTATPTHCAYDRIVVAGMLLQGAVVPDSALPFN
FQAAYGLSDQLAQAISDHYPVEVMLKGSSDKTHISPPSPAPELLGGPSVFLFPPKPKDTLYITR
EPEVTCVVVDVSHEDPEVKENWYVDGVEVHNAKTKPREEQYNSTYRVVSVLTVLHQDWLNGKEY
KCKVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTQKSLSLSPGK

In one aspect, the present disclosure generally relates to a construct comprising the amino acid sequence: W-X-Y-Z; wherein W is a signal sequence; X is a DNAse1 polypeptide or a DNAse1L3 polypeptide; Y is a LINKER; and Z is an Fc domain.

In some aspects, the signal sequence W comprises MRYTGLMGTLLTLVNLLQLAGT (SEQ ID NO: 57). In some aspects, the signal sequence W comprises MSLHPASPRLASLLLFILALHDTLA (SEQ ID NO: 58). In some aspects, the signal sequence W comprises MRGMKLLGALLALAALLQGAVS (SEQ ID NO: 59). In some aspects, the signal sequence W comprises MSRELAPLLLLLLSIHSALA (SEQ ID NO: 60). In some aspects, the signal sequence W comprises MTRLTVLALLAGLLASSRA (SEQ ID NO: 61).

In some aspects, the DNAse1 polypeptide X comprises a mouse DNAse1 polypeptide or a human DNAse1 polypeptide. In some aspects, the DNAse1L3 polypeptide X comprises a mouse DNAse1L3 or a human DNAse1L3 polypeptide.

In some aspects, the DNAse1 polypeptide X comprises a mouse DNAse1 of SEQ ID NO: 29. In some aspects, the DNAse1 polypeptide X comprises a mouse DNAse1 of SEQ ID NO: 29 with E35R, A136F mutations and optionally a deletion of amino acids 1-29 relative to SEQ ID NO: 29. In some aspects, the DNAse1 polypeptide X comprises a mouse DNAse1 of SEQ ID NO: 29 with a V88N mutation relative to SEQ ID NO: 29 and optionally a deletion of amino acids 1-29 relative to SEQ ID NO: 29. In some aspects, the DNAse1 polypeptide X comprises a mouse DNAse1 of SEQ ID NO: 29 with E35R, A136F, G262N mutations relative to SEQ ID NO: 29 and optionally a deletion of amino acids 1-29 relative to SEQ ID NO: 29. In some aspects, the DNAse1 polypeptide X comprises a mouse DNAse1 of SEQ ID NO: 29 with E35R, V88N, G262N mutations relative to SEQ ID NO: 29 and optionally a deletion of amino acids 1-29 relative to SEQ ID NO: 29. In some aspects, the DNAse1 polypeptide X comprises a mouse DNAse1 of SEQ ID NO: 29 with E35R, V88N, L267T mutations relative to SEQ ID NO: 29 and optionally a deletion of amino acids 1-29 relative to SEQ ID NO: 29. In some aspects, the DNAse1 polypeptide X comprises a mouse DNAse1L3 of SEQ ID NO: 30 with A38R, N101K mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, I72T, N101K mutations relative to SEQ ID NO: 30 and optionally a deletion of amino acids 1-25 relative to SEQ ID NO: 30.

In some aspects, the DNAse1 polypeptide X comprises a mouse DNAse1L3 of SEQ ID NO: 30 with A38R, V93T, N101K mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, E226N mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, G130E, T132N, V134T mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, S257N, V259T mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, S257N, V259T, R309H mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, S257N, V259T, R304K mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 with A38R, N101K, S257N, V259T, N295S, R296K mutations relative to SEQ ID NO: 30 and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with N101K, S257N, V259T mutations relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with N101K, mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with I72T mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with V93T, N101K mutations relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with E226N mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with A38R mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with E45T mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with V93T mutation relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with A38R, E226N mutations relative to SEQ ID NO: 30; and optionally a deletion of amino acids 1-25 relative to SEQ ID NO: 30. In some aspects, the DNAse1 polypeptide X comprises a mouse DNAse1L3 of SEQ ID NO: 30 wherein residues 297-316 of SEQ ID NO: 30 are deleted, and also with A38R, E226N, S257N, V259T mutations relative to SEQ ID NO: 30; and optionally a deletion of amino acids 1-25 relative to SEQ ID NO: 30.

In some aspects, the DNAse1 polypeptide X comprises a human DNAse1 of SEQ ID NO: 1. In some aspects, the DNAse1 polypeptide X comprises a human DNAse1 of SEQ ID NO: 1 with Q31R, E35R, N96K, A136F mutations and optionally a deletion of amino acids 1-22 relative to SEQ ID NO: 1. In some aspects, the DNAse1 polypeptide X comprises a human DNAse1 of SEQ ID NO: 1 with Q31R, E35R, N96K mutations and optionally a deletion of amino acids 1-22 relative to SEQ ID NO: 1. In some aspects, the DNAse1 polypeptide X comprises a human DNAse1 of SEQ ID NO: 1 with Q31R, E35R, V88N, N96K, A136F mutations and optionally a deletion of amino acids 1-22 relative to SEQ ID NO: 1. In some aspects, the DNAse1 polypeptide X comprises a human DNAse1 of SEQ ID NO: 1 with Q31R, N96K, A136F mutations and optionally a deletion of amino acids 1-22 relative to SEQ ID NO: 1. In some aspects, the DNAse1 polypeptide X comprises a human DNAse1 of SEQ ID NO: 1 with E35R, N96K mutations and optionally a deletion of amino acids 1-22 relative to SEQ ID NO: 1. In some aspects, the DNAse1 polypeptide X comprises a human DNAse1 of SEQ ID NO: 1 with Q31R, E35R, V88N, N96K mutations and optionally a deletion of amino acids 1-22 relative to SEQ ID NO: 1. In some aspects, the DNAse1 polypeptide X comprises a human DNAse1 of SEQ ID NO: 1 with Q31R, E35R, N96K, A136F mutations and optionally a deletion of amino acids 1-22 relative to SEQ ID NO: 1. In some aspects, the DNAse1 polypeptide X comprises a human DNAse1 of SEQ ID NO: 1 with Q31R, E35R, N96K, A136F, Q258T mutations and optionally a deletion of amino acids 1-22 relative to SEQ ID NO: 1. In some aspects, the DNAse1 polypeptide X comprises a human DNAse1 of SEQ ID NO: 1 with Q31R, E35R, N96K, A136F, D250N, A252T mutations and optionally a deletion of amino acids 1-22 relative to SEQ ID NO: 1. In some aspects, the DNAse1 polypeptide X comprises a human DNAse1 of SEQ ID NO: 1 with Q31R, N96K, A136F mutations and optionally a deletion of amino acids 1-22 relative to SEQ ID NO: 1. In some aspects, the DNAse1 polypeptide X comprises a human DNAse1 of SEQ ID NO: 1 with Q31R, A136F mutations and optionally a deletion of amino acids 1-22 relative to SEQ ID NO: 1. In some aspects, the DNAse1 polypeptide X comprises a human DNAse1 of SEQ ID NO: 1 with Q31R, N96K, A136F, G262N mutations and optionally a deletion of amino acids 1-22 relative to SEQ ID NO: 1. In some aspects, the DNAse1 polypeptide X comprises a human DNAse1 of SEQ ID NO: 1 with Q31R, A136F, G262N mutations and optionally a deletion of amino acids 1-22 relative to SEQ ID NO: 1. In some aspects, the DNAse1 polypeptide X comprises a human DNAse1 of SEQ ID NO: 1 with Q31R, V88N, G262N mutations and optionally a deletion of amino acids 1-22 relative to SEQ ID NO: 1. In some aspects, the DNAse1 polypeptide X comprises a human DNAse1 of SEQ ID NO: 1 with Q31R, V88N mutations and optionally a deletion of amino acids 1-22 relative to SEQ ID NO: 1. In some aspects, the DNAse1 polypeptide X comprises a human DNAse1 of SEQ ID NO: 1 with Q31R mutation and optionally a deletion of amino acids 1-22 relative to SEQ ID NO: 1.

In some aspects, the DNAse1 polypeptide X comprises a human DNAse1 of SEQ ID NO: 1 with Q31R, V88N, G262N mutations and optionally a deletion of amino acids 1-22 relative to SEQ ID NO: 1. In some aspects, the DNAse1 polypeptide X comprises a human DNAse1 of SEQ ID NO: 1 with Q31R, V88N mutations and optionally a deletion of amino acids 1-22 relative to SEQ ID NO: 1.

In some aspects, the DNAse1 polypeptide X comprises a human DNAse1L3 of SEQ ID NO: 2 with D53R, N96K mutations and optionally a deletion of amino acids 1-20 relative to SEQ ID NO: 2. In some aspects, the DNAse1 polypeptide X comprises a human DNAse1L3 of SEQ ID NO: 2 with D53R, M42T mutations and optionally a deletion of amino acids 1-20 relative to SEQ ID NO: 2. In some aspects, the DNAse1 polypeptide X comprises a human DNAse1L3 of SEQ ID NO: 2 with D53R, V88T mutations and optionally a deletion of amino acids 1-20 relative to SEQ ID NO: 2. In some aspects, the DNAse1 polypeptide X comprises a human DNAse1L3 of SEQ ID NO: 2 with D53R, A127N, V129T mutations and optionally a deletion of amino acids 1-20 relative to SEQ ID NO: 2. In some aspects, the DNAse1 polypeptide X comprises a human DNAse1L3 of SEQ ID NO: 2 with D53R, Y98T mutations and optionally a deletion of amino acids 1-20 relative to SEQ ID NO: 2. In some aspects, the DNAse1 polypeptide X comprises a human DNAse1L3 of SEQ ID NO: 2 with D53R, R95N, N96K mutations and optionally a deletion of amino acids 1-20 relative to SEQ ID NO: 2. In some aspects, the DNAse1 polypeptide X comprises a human DNAse1L3 of SEQ ID NO: 2 with R33E, D53R, N96K mutations and optionally a deletion of amino acids 1-20 relative to SEQ ID NO: 2. In some aspects, the DNAse1 polypeptide X comprises a human DNAse1L3 of SEQ ID NO: 2 with D53R, N96K, T288N mutations and optionally a deletion of amino acids 1-20 relative to SEQ ID NO: 2. In some aspects, the DNAse1 polypeptide X comprises a human DNAse1L3 of SEQ ID NO: 2 with D53R, N96K, V254T mutations and optionally a deletion of amino acids 1-20 relative to SEQ ID NO: 2.

In some aspects, the Y is a LINKER that comprises SSTMVRS (SEQ ID NO:40). In some aspects, the Y is a LINKER that comprises SSTMVGS (SEQ ID NO:41). In some aspects, the Y is a LINKER that comprises GS. In some aspects, the Y is a linker that comprises HS. In some aspects, the Y is a LINKER that comprises SSTMVHS (SEQ ID NO: 56).

In some aspects, the Z is an Fc that comprises SEQ ID NO: 31, wherein the Fc region includes T27Y and T31E mutations relative to SEQ ID NO: 31. In some aspects, the Z is an Fc region that comprises the Fc region of SEQ ID NO: 31 with C2S, C5S, T27Y and T31E mutations relative to SEQ ID NO: 31. In some aspects, the Z is an Fc region that the Fc region of SEQ ID NO: 31 with T27Y and T31E mutations and an addition of residues GSVHPKQHR at the C terminal end, relative to SEQ ID NO: 31. In some aspects, the Z is an Fc region that the Fc region of SEQ ID NO: 31 with T27Y and T31E mutations and an addition of residues GSRGQPGVMGF at the C terminal end, relative to SEQ ID NO: 31. In some aspects, the Z is an Fc region that the Fc region of SEQ ID NO: 31 with T27Y and T31E mutations and an addition of residues GSLSALTPSPSWLKYKAL at the C terminal end, relative to SEQ ID NO: 31. In some aspects, the Z is an Fc region that the Fc region of SEQ ID NO: 31 with T27Y and T31E mutations and an addition of residues GSNNQKITNLKQKVAQLEA at the C terminal end, relative to SEQ ID NO: 31. In some aspects, the Z is an Fc region that the Fc region of SEQ ID NO: 31 with T27Y and T31E mutations and an addition of residues GSCGEAIPMSIPPEVK at the C terminal end, relative to SEQ ID NO: 31. In some aspects, the Z is an Fc region that the Fc region of SEQ ID NO: 31 with T27Y and T31E mutations and an addition of residues GSGKDKYENEDLIKHG at the C terminal end, relative to SEQ ID NO: 31. In some aspects, the Z is an Fc region that the Fc region of SEQ ID NO: 31. In some aspects, the Z is an Fc region that comprises the Fc region of SEQ ID NO: 45 with M33Y, S35T and T37E mutations relative to SEQ ID NO: 45. In some aspects, the Z is an Fc region that comprises the Fc region of SEQ ID NO: 45 with C1S, C7S, C10S, M33Y, S35T and T37E mutations relative to SEQ ID NO: 45.

In some aspects, the construct is Construct 1171, comprising a mouse DNAse1 (X) of SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, Construct 1171 further optionally comprises a deletion of amino acids 1-29 relative to SEQ ID NO: 29.

In some aspects, the construct is Construct 1586 comprising a mouse DNAse1 (X) of SEQ ID NO: 29; a linker (Y) of SSTMVGS (SEQ ID NO:41); and an Fc region (Z) of SEQ ID NO: 31, wherein the Fc region includes T27Y and T31E mutations relative to SEQ ID NO: 31. In some aspects, Construct 1171 further optionally comprises a deletion of amino acids 1-29 relative to SEQ ID NO: 29.

In some aspects, the construct is Construct 1671 comprising a mouse DNAse1 (X) of SEQ ID NO: 29 with E35R, A136F mutations relative to SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1674, comprising a mouse DNAse1 (X) of SEQ ID NO: 29 with a V88N mutation relative to SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1679 comprising a mouse DNAse1 (X) of SEQ ID NO: 29 with E35R, A136F, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1687 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1689 comprising a mouse DNAse1 (X) of SEQ ID NO: 29 with E35R, V88N, L267T, mutations relative to SEQ ID NO: 29; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1770 comprising a mouse DNAse1 (X) of SEQ ID NO: 29 with E35R, A136F, mutations relative to SEQ ID NO: 29; a linker (Y) of SSTMVRS (SEQ ID NO:40); and an Fc region (Z) of SEQ ID NO: 31, wherein the Fc region includes C2S, C5S, T27Y and T31E mutations relative to SEQ ID NO: 31. In some aspects, the construct further optionally comprises a deletion of amino acids 1-29 relative to SEQ ID NO: 29.

In some aspects, the construct is Construct 2046 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSVHPKQHR at the C terminal end, relative to SEQ ID NO: 31. In some aspects, the construct further optionally comprises a deletion of amino acids 1-29 relative to SEQ ID NO: 29.

In some aspects, the construct is Construct 2050 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSRGQPGVMGF at the C terminal end, relative to SEQ ID NO: 31. In some aspects, the construct further optionally comprises a deletion of amino acids 1-29 relative to SEQ ID NO: 29.

In some aspects, the construct is Construct 2052 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSLSALTPSPSWLKYKAL at the C terminal end, relative to SEQ ID NO: 31. In some aspects, the construct further optionally comprises a deletion of amino acids 1-29 relative to SEQ ID NO: 29.

In some aspects, the construct is Construct 2055 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSNNQKITNLKQKVAQLEA at the C terminal end, relative to SEQ ID NO: 31. In some aspects, the construct further optionally comprises a deletion of amino acids 1-29 relative to SEQ ID NO: 29.

In some aspects, the construct is Construct 2064 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSCGEAIPMSIPPEVK at the C terminal end, relative to SEQ ID NO: 31. In some aspects, the construct further optionally comprises a deletion of amino acids 1-29 relative to SEQ ID NO: 29.

In some aspects, the construct is Construct 2087 comprising a mouse DNAse1 (X) of SEQ ID NO: 29 with E35R, V88N, G262N mutations relative to SEQ ID NO: 29; a linker (Y) of SSTMVRS (SEQ ID NO:40); and an Fc region (Z) of SEQ ID NO: 31, wherein the Fc region includes T27Y and T31E mutations and an addition of residues GSGKDKYENEDLIKHG at the C terminal end, relative to SEQ ID NO: 31. In some aspects, the construct further optionally comprises a deletion of amino acids 1-29 relative to SEQ ID NO: 29.

In some aspects, the construct is Construct 1720 comprising SEQ ID NO: 29 with two copies of residues 288-310 of SEQ ID NO: 30 appended to the C terminus of SEQ ID NO: 29 (X); a linker (Y) of STMVGS (SEQ ID NO: 41); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1714 comprising SEQ ID NO: 29 with residues 288-295 of SEQ ID NO: 30 appended to the C terminus of SEQ ID NO: 29 (X); a linker (Y) of SSTMVGS (SEQ ID NO: 41); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1725 comprising SEQ ID NO: 29 with residues 288-310 of SEQ ID NO: 30 appended to the C terminus of SEQ ID NO: 29 (X); a linker (Y) of STMVGS (SEQ ID NO: 41); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1727 comprising SEQ ID NO: 29 with two copies of residues 288-295 of SEQ ID NO: 30 appended to the C terminus of SEQ ID NO: 29 (X); a linker (Y) of SSTMVGS (SEQ ID NO: 41); and an Fc region (Z) of SEQ ID NO: 31, wherein the Fc region includes T27Y and T31E mutations relative to SEQ ID NO: 31. In some aspects, the construct further optionally comprises a deletion of amino acids 1-29 relative to SEQ ID NO: 29.

In some aspects, the construct is Construct 1176, comprising a mouse DNAse1L3 (X) of SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1615, comprising a mouse DNAse1L3 (X) of SEQ ID NO: 30 with A38R, N101K mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1656, comprising a mouse DNAse1L3 (X) of SEQ ID NO: 30 with A38R, I72T, N101K mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1658, comprising a mouse DNAse1L3 (X) of SEQ ID NO: 30 with A38R, V93T, N101K mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1659, comprising a mouse DNAse1L3 (X) of SEQ ID NO: 30 with A38R, N101K, E226N mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1662, comprising a mouse DNAse1L3 (X) of SEQ ID NO: 30 with A38R, N101K, G130E, T132N, V134T mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1669, comprising a mouse DNAse1L3 (X) of SEQ ID NO: 30 with A38R, N101K, S257N, V259T mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1772, comprising a mouse DNAse1L3 (X) of SEQ ID NO: 30 with A38R, N101K, S257N, V259T mutations relative to SEQ ID NO: 30; a linker (Y) of SSTMVRS (SEQ ID NO:40); and an Fc region (Z) of SEQ ID NO: 31, wherein the Fc region includes C2S, C5S, T27Y and T31E mutations relative to SEQ ID NO: 31. In some aspects, the construct further optionally comprises a deletion of amino acids 1-25 relative to SEQ ID NO: 30.

In some aspects, the construct is Construct 1782, comprising a mouse DNAse1L3 (X) of SEQ ID NO: 30 with a deletion of the C-terminal tail relative to SEQ ID NO: 30; a linker (Y) of SSTMVRS (SEQ ID NO:40); and an Fc region (Z) of SEQ ID NO: 31. In some aspects, the construct further optionally comprises a deletion of amino acids 1-25 relative to SEQ ID NO: 30.

In some aspects, the construct is Construct 1783, comprising a mouse DNAse1L3 (X) of SEQ ID NO: 30; a linker (Y) of SSTMVRS (SEQ ID NO:40); and an Fc region (Z) of SEQ ID NO: 31, wherein the Fc region includes a N-terminal tail at its C terminus. In some aspects, the construct further optionally comprises a deletion of amino acids 1-25 relative to SEQ ID NO: 30.

In some aspects, the construct is Construct 1966, comprising a mouse DNAse1L3 (X) of SEQ ID NO: 30 with A38R, N101K, S257N, V259T, R309H mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1968, comprising a mouse DNAse1L3 (X) of SEQ ID NO: 30 with A38R, N101K, S257N, V259T, R304K mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1970, comprising a mouse DNAse1L3 (X) of SEQ ID NO: 30 with A38R, N101K, S257N, V259T, N295S, R296K mutations relative to SEQ ID NO: 30; a linker (Y) of SSTMVRS (SEQ ID NO:40); and an Fc region of SEQ ID NO: 31, wherein the Fc region (Z) includes T27Y and T31E mutations relative to SEQ ID NO: 31. In some aspects, the construct further optionally comprises a deletion of amino acids 1-25 relative to SEQ ID NO: 30.

In some aspects, the construct is Construct 1558, comprising pFUSE-mIgG1-Fc-mDnase1L3, comprising an Fc region of SEQ ID NO: 31, wherein the Fc region includes T27Y and T31E mutations relative to SEQ ID NO: 31; a linker of SSTMVRS (SEQ ID NO:40), or of GS, or of HS, or of SSTMVHS (SEQ ID NO: 56); and a mouse DNAse1L3 of SEQ ID NO: 30. In some aspects, the construct further optionally comprises a deletion of amino acids 1-25 relative to SEQ ID NO: 30.

In some aspects, the construct is Construct 1596, comprising a mouse DNAse1L3 (X) of SEQ ID NO: 30, wherein residues 297-316 of SEQ ID NO: 30 are deleted; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1622, comprising a mouse DNAse1L3 (X) of SEQ ID NO: 30, wherein residues 297-316 of SEQ ID NO: 30 are deleted, and also with N101K, S257N, V259T mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1624, comprising a mouse DNAse1L3 (X) of SEQ ID NO: 30, wherein residues 297-316 of SEQ ID NO: 30 are deleted, and also with N101K, mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1628, comprising a mouse DNAse1L3 (X) of SEQ ID NO: 30, wherein residues 297-316 of SEQ ID NO: 30 are deleted, and also with I72T mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1630, comprising a mouse DNAse1L3 (X) of SEQ ID NO: 30, wherein residues 297-316 of SEQ ID NO: 30 are deleted, and also with V93T, N101K mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1632, comprising a mouse DNAse1L3 (X) of SEQ ID NO: 30, wherein residues 297-316 of SEQ ID NO: 30 are deleted, and also with E226N mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1637, comprising a mouse DNAse1L3 (X) of SEQ ID NO: 30, wherein residues 297-316 of SEQ ID NO: 30 are deleted, and also with A38R mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1639, comprising a mouse DNAse1L3 (X) of SEQ ID NO: 30, wherein residues 297-316 of SEQ ID NO: 30 are deleted, and also with E45T mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1643, comprising a mouse DNAse1L3 (X) of SEQ ID NO: 30, wherein residues 297-316 of SEQ ID NO: 30 are deleted, and also with V93T mutation relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1645, comprising a mouse DNAse1L3 (X) of SEQ ID NO: 30, wherein residues 297-316 of SEQ ID NO: 30 are deleted, and also with A38R, E226N mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1667, comprising a mouse DNAse1L3 (X) of SEQ ID NO: 30, wherein residues 297-316 of SEQ ID NO: 30 are deleted, and also with A38R, E226N, S257N, V259T mutations relative to SEQ ID NO: 30; a linker (Y) of SSTMVRS (SEQ ID NO:40); and an Fc region (Z) of SEQ ID NO: 31, wherein the Fc region includes T27Y and T31E mutations relative to SEQ ID NO: 31. In some aspects, the construct further optionally comprises a deletion of amino acids 1-25 relative to SEQ ID NO: 30.

In some aspects, the construct is Construct 1825, which construct comprises Q31R, E35R, N96K, A136F, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1830, which construct comprises Q31R, E35R, N96K, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1832, which construct comprises Q31R, E35R, V88N, N96K, A136F, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1833, which construct comprises Q31R, N96K, A136F, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1837, which construct comprises E35R, N96K, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1838, which construct comprises Q31R, E35R, V88N, N96K, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1848, which construct comprises Q31R, E35R, N96K, A136F, T288N, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1852, which construct comprises Q31R, E35R, N96K, A136F, Q258T, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1854, which construct comprises Q31R, E35R, N96K, A136F, D250N, A252T, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2007, which construct comprises Q31R, N96K, A136F, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2015, which construct comprises Q31R, A136F, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2017, which construct comprises Q31R, N96K, A136F, G262N, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2021, which construct comprises Q31R, A136F, G262N, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2023, which construct comprises Q31R, V88N, G262N, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2026, which construct comprises Q31R, V88N, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2027, which construct comprises Q31R, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2029, which construct comprises Q31R, V88N, G262N, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 2033, which construct comprises Q31R, V88N, C285S, C291S, C294S, M317Y, S319T, T321E mutations relative to SEQ ID NO: 46. In some aspects, the construct further optionally comprises a deletion of amino acids 1-20 relative to SEQ ID NO: 46.

In some aspects, the construct is Construct 1828, which construct comprises a human DNAse1L3 (X) of SEQ ID NO: 2 with E53R, N96K mutations relative to SEQ ID NO: 2; a GS linker (Y); and an Fc region (Z) of SEQ ID NO: 45, wherein the Fc region includes M33Y, S35T, and T37E mutations relative to SEQ ID NO: 45. In some aspects, the construct further optionally comprises a deletion of amino acids 1-20 relative to SEQ ID NO: 2.

In some aspects, the construct is Construct 1857, which construct comprises a human DNAse1L3 (X) of SEQ ID NO: 2 with E53R, M42T mutations relative to SEQ ID NO: 2; a GS linker (Y); and an Fc region (Z) of SEQ ID NO: 45, wherein the Fc region includes M33Y, S35T, T37E mutations relative to SEQ ID NO: 45. In some aspects, the construct further optionally comprises a deletion of amino acids 1-20 relative to SEQ ID NO: 2.

In some aspects, the construct is Construct 1858, which construct comprises a human DNAse1L3 (X) of SEQ ID NO: 2 with E53R, V88T mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1859, which construct comprises a human DNAse1L3 (X) of SEQ ID NO: 2 with E53R, A127N, V129T mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1860, which construct comprises a human DNAse1L3 (X) of SEQ ID NO: 2 with E53R, Y98T mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1861, which construct comprises a human DNAse1L3 (X) of SEQ ID NO: 2 with E53R, R95N, N96K mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1862, which construct comprises a human DNAse1L3 (X) of SEQ ID NO: 2 with R33E, E53R, N96K mutations relative to SEQ ID NO: 2; a GS linker (Y); and an Fc region of SEQ ID NO: 45, wherein the Fc region (Z) includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1865, which construct comprises a human DNAse1L3 (X) of SEQ ID NO: 2 with E53R, N96K, T288N mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In some aspects, the construct is Construct 1866, which construct comprises a human DNAse1L3 (X) of SEQ ID NO: 2 with E53R, N96K, V254T mutations relative to SEQ ID NO: 2; a GS linker (Y); and an Fc region (Z) of SEQ ID NO: 45, wherein the Fc region includes M33Y, S35T and T37E mutations relative to SEQ ID NO: 45. In some aspects, the construct further optionally comprises a deletion of amino acids 1-20 relative to SEQ ID NO: 2.

In certain embodiments, the present disclosure contemplates a construct that is expressed from a mammalian cell line, such as but not limited to a CHO cell line, which is stably transfected with human ST6 beta-galactosamide alpha-2,6-sialyltransferase (ST6GAL1). In certain embodiments, such expression enhances sialyation of the construct. The present disclosure further provides a construct that is grown in a cell culture supplemented with sialic acid and/or N-acetylmannosamine (1,3,4-O-Bu3ManNAc). In certain embodiments, such growth enhances sialic acid capping of the construct.

In certain embodiments, enhancing protein sialyation by expressing the biologic in CHO cells stably transfected with human alpha-2,6-sialyltransferase substantially improved construct bioavailability (Cₘₐₓ) when dosed subcutaneously. In other embodiments, increasing the pH-dependent FcRn-mediated cellular recycling by manipulating the Fc domain led to improvements of in vivo biologic half-life. In yet other embodiments, combining CHO cells stably transfected with human α-2,6-sialyltransferase and growing the cells in N-acetylmannosamine led to dramatic increases half-life and/or biologic exposure (AUC). In yet other embodiments, combining two or more methods described herein into a single construct led to dramatic increases in half-life and/or biologic exposure (AUC).

In certain embodiments, the constructs of the disclosure are more highly glycosylated than other DNAse1 and/or DNAse1L3 constructs in the art. In other embodiments, the constructs of the disclosure have higher affinity for the neonatal orphan receptor (FcRn) than other DNAse1 and/or DNAse1L3 constructs in the art. In yet other embodiments, the constructs of the disclosure have higher in vivo half-lives than other DNAse1 and/or DNAse1L3 constructs in the art. In yet other embodiments, the in vivo half-life of a construct of the disclosure is at least about 1.5, 2, 2.5, 3, 4, 5, 6, 7, 8, 9, 10, 12, 14, 16, 18, or 20 times higher than the DNAse1 and/or DNAse1L3 constructs described in the art. In yet other embodiments, the constructs of the disclosure are administered to the subject at a lower dose and/or at a lower frequency than other DNAse1 and/or DNAse1L3 constructs in the art. In yet other embodiments, the constructs of the disclosure are administered to the subject once a month, twice a month, three times a month, four times a month, once a week, and/or twice a week. In yet other embodiments, the lower frequency administration of the constructs of the disclosure results in better patient compliance and/or increased efficacy as compared with other DNAse1 and/or DNAse1L3 constructs in the art.

In certain embodiments, the construct is soluble. In other embodiments, the construct is a recombinant polypeptide.

In certain embodiments, the construct comprises a signal peptide resulting in the secretion of a precursor of the DNAse1 and/or DNAse1L3 polypeptide, which undergoes proteolytic processing to yield a processed construct comprising the DNAse1 and/or DNAse1L3 polypeptide.

In certain embodiments, the DNAse1 and/or DNAse1L3 polypeptide is C-terminally fused to the Fc domain of human immunoglobulin 1 (IgG1), human immunoglobulin 2 (IgG2), human immunoglobulin 3 (IgG3), and/or human immunoglobulin 4 (IgG4). In other embodiments, the DNAse1 and/or DNAse1L3 polypeptide is N-terminally fused to the Fc domain of human immunoglobulin 1 (IgG1), human immunoglobulin 2 (IgG2), human immunoglobulin 3 (IgG3), and/or human immunoglobulin 4 (IgG4). In yet other embodiments, the presence of IgFc domain improves half-life, solubility, reduces immunogenicity, and increases the activity of the DNAse1 and/or DNAse1L3 polypeptide.

In certain embodiments, the DNAse1 and/or DNAse1L3 polypeptide is C-terminally fused to human serum albumin. Human serum albumin may be conjugated to DNAse1 and/or DNAse1L3 protein through a chemical linker, including but not limited to naturally occurring or engineered disulfide bonds, and/or by genetic fusion to DNAse1 and/or DNAse1L3, and/or a fragment and/or variant thereof.

In certain embodiments, the construct is further pegylated (i.e., fused with a poly(ethylene glycol) chain).

In certain embodiments, the construct is formulated as a liquid formulation. In other embodiments, the disclosure provides a dry and/or lyophilized product form of a pharmaceutical composition comprising a therapeutic amount of a construct of the disclosure, whereby the dry product is reconstitutable to a solution of the construct in liquid form.

The disclosure provides a kit comprising at least one construct of the disclosure, and/or a salt or solvate thereof, and instructions for using the construct within the methods of the disclosure.

It will be understood that a DNAse1 and/or DNAse1L3 polypeptide according to the disclosure includes not only the native human proteins, but also any fragment, derivative, fusion, conjugate or mutant thereof. As used herein in this disclosure, the phrase “a DNAse1 and/or DNAse1L3 polypeptide, mutant, and/or mutant fragment thereof” also includes any compound or polypeptide (such as, but not limited to, a fusion protein) comprising a DNAse1 and/or DNAse1L3 polypeptide, mutant, and/or mutant fragment thereof. Fusion proteins according to the disclosure are considered biological equivalents of DNAse1 and/or DNAse1L3, but can in certain embodiments provide longer half-life or greater potency due to increased in vivo biologic exposure, as judged by the “area under the curve” (AUC) or increased half-life in pharmacokinetic experiments.

### Vectors and Cells

The disclosure further provides an autonomously replicating or an integrative mammalian cell vector comprising a recombinant nucleic acid encoding a polypeptide of the disclosure. In certain embodiments, the vector comprises a plasmid or a virus. In other embodiments, the vector comprises a mammalian cell expression vector. In yet other embodiments, the vector further comprises at least one nucleic acid sequence that directs and/or controls expression of the polypeptide. In yet other embodiments, the recombinant nucleic acid encodes a construct comprising a DNAse1 and/or DNAse1L3 polypeptide and a signal peptide, wherein the polypeptide is proteolytically processed upon secretion from a cell to yield the DNAse1 and/or DNAse1L3 construct of the disclosure.

In yet another aspect, the disclosure provides an isolated host cell comprising a vector of the disclosure. In certain embodiments, the cell is a non-human cell. In other embodiments, the cell is mammalian. In yet other embodiments, the vector of the disclosure comprises a recombinant nucleic acid encoding a construct comprising a DNAse1 and/or DNAse1L3 polypeptide and a signal peptide. In yet other embodiments, the polypeptide is proteolytically processed upon secretion from a cell to yield the DNAse1 and/or DNAse1L3 construct of the disclosure.

Production and Purification of DNAse1 and/or DNAse1L3 Fusion Proteins

In certain embodiments, a soluble DNAse1 and/or DNAse1L3 construct, including IgG Fc domain or enzymatically/biologically active fragments thereof, are efficacious in treating, reducing, and/or preventing progression of diseases or disorders contemplated herein.

To produce soluble, recombinant DNAse1 and/or DNAse1L3 constructs for in vitro use, DNAse1 and/or DNAse1L3 polypeptides can be fused to the Fc domain of IgG (referred to as “DNAse1-Fc” or “DNAse1L3-Fc”) and the fusion construct can be expressed in stable CHO cell lines. The construct can also be expressed from HEK293 cells, Baculovirus insect cell system or CHO cells or Yeast Pichia expression system using suitable vectors. The construct can be produced in either adherent or suspension cells. To establish stable cell lines the nucleic acid sequence encoding DNAse1 and/or DNAse1L3 constructs are cloned into an appropriate vector for large scale protein production.

Many expression systems are known can be used for the production of DNAse1 and/or DNAse1L3 constructs, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired proteins can be produced in conventional ways, for example from a coding sequence inserted in the host chromosome or on a free plasmid.

The yeasts can be transformed with a coding sequence for the desired protein in any one of the usual ways, for example electroporation. Methods for transformation of yeast by electroporation are disclosed in Becker & Guarente, 1990, Methods Enzymol. 194: 182. Successfully transformed cells, i.e., cells that contain a DNA construct of the present disclosure, can be identified by well-known techniques. For example, cells resulting from the introduction of an expression construct can be grown to produce the desired polypeptide. Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available fron1 Strat:1.gene Cloning Systems, La Jolla, CA, USA Plasmids pRS403, pRS404, pRS405 and pRS406 are Yeast Integrating plasmids (Y1ps) and incorporate the yeast selectable markers I-llS3, TRP1, LEU2 and lJRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Clones of single, stably transfected cells are then established and screened for high expressing clones of the desired fusion protein. Screening of the single cell clones for DNAse1 and/or DNAse1L3 protein expression can be accomplished in a high-throughput manner in 96 well plates. Upon identification of high expressing clones through screening, protein production can be accomplished in shaking flasks or bio-reactors.

Purification of DNAse1 and/or DNAse1L3 constructs can be accomplished using a combination of standard purification techniques known in the art.

### Gene Therapy

The nucleic acids encoding the polypeptide(s) useful within the disclosure may be used in gene therapy protocols for the treatment of the diseases or disorders contemplated herein. The improved construct encoding the polypeptide(s) can be inserted into the appropriate gene therapy vector and administered to a patient to treat or prevent the diseases or disorder of interest.

Vectors, such as viral vectors, have been used in the prior art to introduce genes into a wide variety of different target cells. Typically, the vectors are exposed to the target cells so that transformation can take place in a sufficient proportion of the cells to provide a useful therapeutic or prophylactic effect from the expression of the desired polypeptide (e.g., a receptor). The transfected nucleic acid may be permanently incorporated into the genome of each of the targeted cells, providing long lasting effect, or alternatively the treatment may have to be repeated periodically. In certain embodiments, the (viral) vector transfects liver cells in vivo with genetic material encoding the polypeptide(s) of the disclosure.

A variety of vectors, both viral vectors and plasmid vectors are known in the art (see for example U.S. Pat. No. 5,252,479 and WO 93/07282). In particular, a number of viruses have been used as gene transfer vectors, including papovaviruses, such as SV40, vaccinia virus, herpes viruses including HSV and EBV, and retroviruses. Many gene therapy protocols in the prior art have employed disabled murine retroviruses. Several recently issued patents are directed to methods and compositions for performing gene therapy (see for example U.S. Pat. Nos. 6,168,916; 6,135,976; 5,965,541 and 6,129,705). Each of the foregoing patents is incorporated by reference in its entirety herein.

### AAV-Mediated Gene Therapy:

AAV, a parvovirus belonging to the genus Dependovirus, has several features that make it particularly well suited for gene therapy applications. For example, AAV can infect a wide range of host cells, including non-dividing cells. Furthermore, AAV can infect cells from a variety of species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, which is a linear, single-stranded DNA molecule containing approximately 4,700 nucleotides (the AAV-2 genome consists of 4,681 nucleotides, the AAV-4 genome 4,767), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, which allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, that is, it requires co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV replicates in canine cells that have been co-infected with a canine adenovirus.

To produce infectious recombinant AAV (rAAV) containing a heterologous nucleic acid sequence, a suitable host cell line can be transfected with an AAV vector containing the heterologous nucleic acid sequence, but lacking the AAV helper function genes, rep and cap. The AAV-helper function genes can then be provided on a separate vector. Also, only the helper virus genes necessary for AAV production (i.e., the accessory function genes) can be provided on a vector, rather than providing a replication-competent helper virus (such as adenovirus, herpesvirus, or vaccinia).

Collectively, the AAV helper function genes (i.e., rep and cap) and accessory function genes can be provided on one or more vectors. Helper and accessory function gene products can then be expressed in the host cell where they will act in trans on rAAV vectors containing the heterologous nucleic acid sequence. The rAAV vector containing the heterologous nucleic acid sequence will then be replicated and packaged as though it were a wild-type (wt) AAV genome, forming a recombinant virion. When a patient's cells are infected with the resulting rAAV virions, the heterologous nucleic acid sequence enters and is expressed in the patient's cells. Because the patient's cells lack the rep and cap genes, as well as the accessory function genes, the rAAV cannot further replicate and package their genomes. Moreover, without a source of rep and cap genes, wtAAV cannot be formed in the patient's cells.

There are eleven known AAV serotypes, AAV-1 through AAV-11 (Mori, et al., 2004, Virology 330(2):375-83). AAV-2 is the most prevalent serotype in human populations; one study estimated that at least 80% of the general population has been infected with wt AAV-2 (Berns and Linden, 1995, Bioessays 17:237-245). AAV-3 and AAV-5 are also prevalent in human populations, with infection rates of up to 60% (Georg-Fries, et al., 1984, Virology 134:64-71). AAV-1 and AAV-4 are simian isolates, although both serotypes can transduce human cells (Chiorini, et al., 1997, J Virol 71:6823-6833; Chou, et al., 2000, Mol Ther 2:619-623). Of the six known serotypes, AAV-2 is the best characterized. For instance, AAV-2 has been used in a broad array of in vivo transduction experiments, and has been shown to transduce many different tissue types including: mouse (U.S. Pat. Nos. 5,858,351; 6,093,392), dog muscle; mouse liver (Couto, et al., 1999, Proc. Natl. Acad. Sci. USA 96:12725-12730; Couto, et al., 1997, J. Virol. 73:5438-5447; Nakai, et al., 1999, J. Virol. 73:5438-5447; and, Snyder, et al., 1997, Nat. Genet. 16:270-276); mouse heart (Su, et al., 2000, Proc. Natl. Acad. Sci. USA 97:13801-13806); rabbit lung (Flotte, et al., 1993, Proc. Natl. Acad. Sci. USA 90:10613-10617); and rodent photoreceptors (Flannery et al., 1997, Proc. Natl. Acad. Sci. USA 94:6916-6921).

The broad tissue tropism of AAV-2 may be exploited to deliver tissue-specific transgenes. For example, AAV-2 vectors have been used to deliver the following genes: the cystic fibrosis transmembrane conductance regulator gene to rabbit lungs (Flotte, et al., 1993, Proc. Natl. Acad. Sci. USA 90:10613-10617); Factor NIII gene (Burton, et al., 1999, Proc. Natl. Acad. Sci. USA 96:12725-12730) and Factor IX gene (Nakai, et al., 1999, J. Virol. 73:5438-5447; Snyder, et al., 1997, Nat. Genet. 16:270-276; U.S. Pat. No. 6,093,392) to mouse liver, dog, and mouse muscle (U.S. Pat. No. 6,093,392); erythropoietin gene to mouse muscle (U.S. Pat. No. 5,858,351); vascular endothelial growth factor (VEGF) gene to mouse heart (Su, et al., 2000, Proc. Natl. Acad. Sci. USA 97:13801-13806); and aromatic 1-amino acid decarboxylase gene to monkey neurons. Expression of certain rAAV-delivered transgenes has therapeutic effect in laboratory animals; for example, expression of Factor IX was reported to have restored phenotypic normalcy in dog models of hemophilia B (U.S. Pat. No. 6,093,392). Moreover, expression of rAAV-delivered NEGF to mouse myocardium resulted in neovascular formation (Su, et al., 2000, Proc. Natl. Acad. Sci. USA 97:13801-13806), and expression of rAAV-delivered AADC to the brains of parkinsonian monkeys resulted in the restoration of dopaminergic function.

Delivery of a protein of interest to the cells of a mammal is accomplished by first generating an AAV vector comprising DNA encoding the protein of interest and then administering the vector to the mammal. Thus, the disclosure should be construed to include AAV vectors comprising DNA encoding the polypeptide(s) of interest. Once armed with the present disclosure, the generation of AAV vectors comprising DNA encoding this/these polypeptide(s)s will be apparent to the skilled artisan.

In certain embodiments, the rAAV vector of the disclosure comprises several essential DNA elements. In certain embodiments, these DNA elements include at least two copies of an AAV ITR sequence, a promoter/enhancer element, a transcription termination signal, any necessary 5′ or 3′ untranslated regions which flank DNA encoding the protein of interest or a biologically active fragment thereof. The rAAV vector of the disclosure may also include a portion of an intron of the protein on interest. Also, optionally, the rAAV vector of the disclosure comprises DNA encoding a mutated polypeptide of interest.

In certain embodiments, the vector comprises a promoter/regulatory sequence that comprises a promiscuous promoter which is capable of driving expression of a heterologous gene to high levels in many different cell types. Such promoters include, but are not limited to the cytomegalovirus (CMV) immediate early promoter/enhancer sequences, the Rous sarcoma virus promoter/enhancer sequences and the like. In certain embodiments, the promoter/regulatory sequence in the rAAV vector of the disclosure is the CMV immediate early promoter/enhancer. However, the promoter sequence used to drive expression of the heterologous gene may also be an inducible promoter, for example, but not limited to, a steroid inducible promoter, or may be a tissue specific promoter, such as, but not limited to, the skeletal α-actin promoter which is muscle tissue specific and the muscle creatine kinase promoter/enhancer, and the like.

In certain embodiments, the rAAV vector of the disclosure comprises a transcription termination signal. While any transcription termination signal may be included in the vector of the disclosure, in certain embodiments, the transcription termination signal is the SV40 transcription termination signal.

In certain embodiments, the rAAV vector of the disclosure comprises isolated DNA encoding the polypeptide of interest, or a biologically active fragment of the polypeptide of interest. The disclosure should be construed to include any mammalian sequence of the polypeptide of interest, which is either known or unknown. Thus, the disclosure should be construed to include genes from mammals other than humans, which polypeptide functions in a substantially similar manner to the human polypeptide. Preferably, the nucleotide sequence comprising the gene encoding the polypeptide of interest is about 50% homologous, more preferably about 70% homologous, even more preferably about 80% homologous and most preferably about 90% homologous to the gene encoding the polypeptide of interest.

Further, the disclosure should be construed to include naturally occurring variants or recombinantly derived mutants of wild type protein sequences, which variants or mutants render the polypeptide encoded thereby either as therapeutically effective as full-length polypeptide, or even more therapeutically effective than full-length polypeptide in the gene therapy methods of the disclosure.

The disclosure should also be construed to include DNA encoding variants which retain the polypeptide's biological activity. Such variants include proteins or polypeptides which have been or may be modified using recombinant DNA technology, such that the protein or polypeptide possesses additional properties which enhance its suitability for use in the methods described herein, for example, but not limited to, variants conferring enhanced stability on the protein in plasma and enhanced specific activity of the protein. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function.

The disclosure is not limited to the specific rAAV vector exemplified in the experimental examples; rather, the disclosure should be construed to include any suitable AAV vector, including, but not limited to, vectors based on AAV-1, AAV-3, AAV-4 and AAV-6, and the like.

Also included in the disclosure is a method of treating a mammal having a disease or disorder in an amount effective to provide a therapeutic effect. The method comprises administering to the mammal an rAAV vector encoding the polypeptide of interest. Preferably, the mammal is a human.

Typically, the number of viral vector genomes/mammal which are administered in a single injection ranges from about 1×10⁸ to about 5×10¹⁶. Preferably, the number of viral vector genomes/mammal which are administered in a single injection is from about 1×10¹⁰ to about 1×10¹⁵; more preferably, the number of viral vector genomes/mammal which are administered in a single injection is from about 5×10¹⁰ to about 5×10¹⁵; and, most preferably, the number of viral vector genomes which are administered to the mammal in a single injection is from about 5×10¹¹ to about 5×10¹⁴.

When the method of the disclosure comprises multiple site simultaneous injections, or several multiple site injections comprising injections into different sites over a period of several hours (for example, from about less than one hour to about two or three hours) the total number of viral vector genomes administered may be identical, or a fraction thereof or a multiple thereof, to that recited in the single site injection method.

For administration of the rAAV vector of the disclosure in a single site injection, in certain embodiments a composition comprising the virus is injected directly into an organ of the subject (such as, but not limited to, the liver of the subject).

For administration to the mammal, the rAAV vector may be suspended in a pharmaceutically acceptable carrier, for example, HEPES buffered saline at a pH of about 7.8. Other useful pharmaceutically acceptable carriers include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The rAAV vector of the disclosure may also be provided in the form of a kit, the kit comprising, for example, a freeze-dried preparation of vector in a dried salts formulation, sterile water for suspension of the vector/salts composition and instructions for suspension of the vector and administration of the same to the mammal.

### Methods

The disclosure includes a method of treating, ameliorating, and/or preventing forms of lupus (including SLE) associated with DNAse1L3 deficiency.

The disclosure includes a method of treating, ameliorating, and/or preventing diseases and/or disorders associated with inefficient NET hydrolysis (“NETolysis”).

The disclosure includes a method of treating, ameliorating, and/or preventing autoimmune disorders. In certain embodiments, the autoimmune disorders comprise lupus (including SLE), thyroid autoimmune disease, and/or Hypocomplementeric Urticarial Vasculitis Syndrome (HUVS).

The disclosure includes a method of treating, ameliorating, and/or preventing pathologic thrombosis, such as but not limited to microvascular thrombosis, venous thrombosis, and/or arterial thrombosis. In certain embodiments, the pathologic thrombosis comprises neutrophilic thrombosis, which includes but is not limited to Anti-Neutrophilic Cytoplasmic Autoantibodies (ANCA) vasculitis, Thrombotic thrombocytopenic purpura (TTP), and Bechet's (or Behcet's) disease or syndrome. In certain embodiments, the pathologic thrombosis comprises thrombosis leading to strokes.

The disclosure includes a method of treating, ameliorating, and/or preventing myocardial infarctions.

The disclosure includes a method of treating, ameliorating, and/or preventing spread and progression of cancer (e.g., cancer metastasis).

The disclosure includes a method of treating, ameliorating, or preventing inefficient NET hydrolysis (“NETolysis”) in a subject afflicted with a bacterial and/or viral infection. The disclosure further includes a method of treating, ameliorating, or preventing systemic inflammation, organ damage and/or sepsis in a subject afflicted with a bacterial and/or viral infection.

The disclosure provides a method of treating, ameliorating, and/or preventing lung injury, such as but not limited to acute lung injury (ALI), in a subject afflicted with acute kidney injury (AKI). In certain embodiments, the ALI comprises at least one of non-cardiogenic pulmonary edema, impaired lung fluid clearance, and respiratory failure requiring mechanical ventilation.

The disclosure provides a method of reducing, reversing increase of, and/or suppressing levels of IL-6 in a subject.

The disclosure provides a method of reducing, reversing increase of, and/or suppressing levels of IL-6 in a subject afflicted with acute kidney injury (AKI).

The disclosure provides a method of treating, ameliorating, and/or preventing Diffuse Alveolar Hemorrhage (DAH) in a subject.

In certain embodiments, the subject suffers from and/or is afflicted by ANCA vasculitis. In certain embodiments, the subject suffers from and/or is afflicted by Goodpasture's disease. In certain embodiments, the subject suffers from and/or is afflicted by (systemic) lupus erythematosus (SLE). In certain embodiments, the subject suffers from and/or is afflicted by antiphospholipid syndrome. In certain embodiments, the subject suffers from and/or is afflicted by Lane-Hamilton syndrome. In certain embodiments, the subject suffers from and/or is afflicted by post-sepsis. In certain embodiments, the subject has undergone bone marrow and/or organ transplant. In certain embodiments, the subject has undergone exposure to cytotoxic and non-cytotoxic drugs. In certain embodiments, the subject suffers from and/or is afflicted by acute kidney injury. In certain embodiments, the subject suffers from and/or is afflicted by viral and/or bacterial infection. In certain embodiments, the subject suffers from and/or is afflicted by poststreptococcal glomerulonephritis. In certain embodiments, the subject suffers from and/or is afflicted by bacterial endocarditis. In certain embodiments, the subject suffers from and/or is afflicted by SARS-CoV-2 infection (COVID-19).

In certain embodiments, the administering reduces, minimizes, and/or prevents mortality due to DAH.

In certain embodiments, the subject requires intensive care unit (ICU) treatment. In certain embodiments, the administering reduces ICU time for the subject.

In certain embodiments, the administering reduces, minimizes, and/or prevents DAH morbidity in the subject.

The disclosure provides a method of treating, ameliorating, and/or preventing Aicardi-Goutieres Syndrome (AGS) in a subject.

In certain embodiments, the AGS-afflicted subject has “classic” AGS with prenatal or infantile onset. In certain embodiments, the AGS-afflicted subject has subacute onset of profound neurological regression, a variable combination of spasticity and dystonia associated with normal neuroimaging, and/or specific white matter changes and/or intracranial calcification. In certain embodiments, the AGS-afflicted subject has bilateral striatal necrosis. In certain embodiments, the AGS-afflicted subject has progressive (‘non-syndromic’) spastic paraparesis confined to at least one lower limb. In certain embodiments, the AGS-afflicted subject has intracerebral, large vessel disease. In certain embodiments, the AGS-afflicted subject has chilblain like lesions of the skin.

In certain embodiments, the method comprises administering a construct of the disclosure to the subject who is suffering from, suspect of suffering from, and/or likely to develop any disease or disorder contemplated herein.

In certain embodiments, the construct of the disclosure is a secreted product of a DNAse1 and/or DNAse1L3 precursor construct (which is itself a construct contemplated within the disclosure) expressed in a mammalian cell. In other embodiments, the DNAse1 and/or DNAse1L3 precursor construct comprises a signal peptide sequence and a DNAse1 and/or DNAse1L3 polypeptide, wherein the DNAse1 and/or DNAse1L3 precursor construct undergoes proteolytic processing to a processed construct comprising the DNAse1 and/or DNAse1L3 polypeptide. In yet other embodiments, in the DNAse1 and/or DNAse1L3 precursor construct the signal peptide sequence is conjugated to the DNAse1 and/or DNAse1L3 polypeptide N-terminus. Upon proteolysis, the signal sequence is cleaved from the DNAse1 and/or DNAse1L3 precursor construct to provide the construct comprising the DNAse1 and/or DNAse1L3 polypeptide.

In certain embodiments, the construct is administered acutely or chronically to the subject. In other embodiments, the construct is administered locally, regionally, parenterally, or systemically to the subject.

In certain embodiments, the subject is a mammal. In other embodiments, the mammal is human.

In certain embodiments, the construct, and/or its precursor construct, is administered by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical. In other embodiments, the construct, and/or its precursor construct, is administered to the subject as a pharmaceutical composition further comprising at least one pharmaceutically acceptable carrier.

In certain embodiments, the construct, and/or its precursor construct, is administered acutely or chronically to the subject. In other embodiments, the construct, and/or its precursor construct, is administered locally, regionally, or systemically to the subject. In yet another embodiment, the construct, and/or its precursor construct, is delivered on an encoded vector, wherein the vector encodes the protein and is transcribed and translated from the vector upon administration of the vector to the subject.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present disclosure may provide benefit.

Thus, the present disclosure, as described more fully herein, includes a method for preventing diseases and disorders in a subject, in that a polypeptide or construct of the disclosure, as discussed elsewhere herein, can be administered to a subject prior to the onset of the disease or disorder, thereby preventing the disease or disorder from developing. Particularly, where the symptoms of the disease or disorder have not manifested to the point of detriment to the subject; indeed, the disease or disorder need not be detected in a subject before treatment is administered. That is, significant pathology from the disease or disorder does not have to occur before the present disclosure may provide benefit. Therefore, the present disclosure includes methods for preventing or delaying onset, and/or reducing progression or growth, of a disease or disorder in a subject, in that a polypeptide of the disclosure can be administered to a subject prior to detection of the disease or disorder. In certain embodiments, the polypeptide of the disclosure is administered to a subject with a strong family history of the disease or disorder, thereby preventing or delaying onset or progression of the disease or disorder.

Armed with the disclosure herein, one skilled in the art would thus appreciate that the prevention of a disease or disorder in a subject encompasses administering to a subject a polypeptide of the disclosure as a preventative measure against the disease or disorder.

### Pharmaceutical Compositions and Formulations

The disclosure provides pharmaceutical compositions comprising a polypeptide of the disclosure within the methods described herein.

Such a pharmaceutical composition is in a form suitable for administration to a subject, and/or the pharmaceutical composition may further comprise one or more pharmaceutically acceptable carriers, one or more additional ingredients, and/or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the disclosure may be administered to deliver a dose of between 1 ng/kg/day and 100 mg/kg/day. In other embodiments, the pharmaceutical compositions useful for practicing the disclosure may be administered to deliver a dose of between 1 ng/kg/day and 500 mg/kg/day.

The relative amounts of the active ingredient, the pharmaceutically acceptable carrier, and any additional ingredients in a pharmaceutical composition of the disclosure will vary, depending upon the identity, size, and condition of the subject treated and further depending upon the route by which the composition is to be administered. By way of example, the composition may comprise between about 0.1% and about 100% (w/w) active ingredient.

Pharmaceutical compositions that are useful in the methods of the disclosure may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

### Administration/Dosing

The regimen of administration may affect what constitutes an effective amount. For example, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. Dosage is determined based on the biological activity of the therapeutic compound which in turn depends on the half-life and the area under the plasma time of the therapeutic compound curve. The polypeptide according to the disclosure can be administered at an appropriate time interval of every 2 days, or every 4 days, or every week or every month. Therapeutic dosage of the polypeptides of the disclosure may also be determined based on half-life or the rate at which the therapeutic polypeptide is cleared out of the body. The polypeptide according to the disclosure is administered at appropriate time intervals of either every 2 days, or every 4 days, every week, or every month to achieve a constant level of enzymatic activity of DNAse1 and/or DNAse1L3.

For example, several divided doses may be administered daily, or the dose may be proportionally reduced as indicated by the exigencies of the therapeutic situation. A non-limiting example of an effective dose range for a therapeutic compound of the disclosure is from about 0.01 and 50 mg/kg of body weight/per day. In some embodiments, the effective dose range for a therapeutic compound of the disclosure is from about 50 ng to 500 ng/kg, preferably 100 ng to 300 ng/kg of bodyweight. One of ordinary skill in the art would be able to study the relevant factors and make the determination regarding the effective amount of the therapeutic compound without undue experimentation.

The compound can be administered to a patient as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the patient.

Actual dosage levels of the active ingredients in the pharmaceutical compositions of this disclosure may be varied to obtain an amount of the active ingredient that is effective to achieve the desired therapeutic response for a particular patient, composition, and mode of administration, without being toxic to the patient.

A medical doctor, e.g., physician, having ordinary skill in the art may readily determine and prescribe the effective amount of the pharmaceutical composition required. For example, the physician or veterinarian could start doses of the compounds of the disclosure employed in the pharmaceutical composition at levels lower than that required to achieve the desired therapeutic effect and gradually increase the dosage until the desired effect is achieved.

In certain embodiments, the compositions of the disclosure are administered to the patient in dosages that range from one to five times per day or more. In other embodiments, the compositions of the disclosure are administered to the patient in range of dosages that include, but are not limited to, once every day, every two, days, every three days to once a week, and once every two weeks. The frequency of administration of the various combination compositions of the disclosure varies from subject to subject depending on many factors including, but not limited to, age, disease, or disorder to be treated, gender, overall health, and other factors. Thus, the disclosure should not be construed to be limited to any particular dosage regime and the precise dosage and composition to be administered to any patient will be determined by the attending physical taking all other factors about the patient into account.

In certain embodiments, the present disclosure is directed to a packaged pharmaceutical composition comprising a container holding a therapeutically effective amount of a compound of the disclosure, alone or in combination with a second pharmaceutical agent; and instructions for using the compound to treat, prevent, or reduce one or more symptoms of a disease or disorder in a patient.

### Routes of Administration

Routes of administration of any one of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. The formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Additional Administration Forms

Additional dosage forms of this disclosure include dosage forms as described in U.S. Pat. Nos. 6,340,475, 6,488,962, 6,451,808, 5,972,389, 5,582,837, and 5,007,790. Additional dosage forms of this disclosure also include dosage forms as described in U.S. Patent Applications Nos. 20030147952, 20030104062, 20030104053, 20030044466, 20030039688, and 20020051820. Additional dosage forms of this disclosure also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

Controlled- or sustained-release formulations of a pharmaceutical composition of the disclosure may be made using conventional technology. In some cases, the dosage forms to be used can be provided as slow or controlled release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Single unit dosage forms suitable for oral administration, such as tablets, capsules, gelcaps, and caplets, which are adapted for controlled release are encompassed by the present disclosure.

In certain embodiments, the formulations of the present disclosure may be, but are not limited to, short-term, rapid offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form. For sustained release, the compounds may be formulated with a suitable polymer or hydrophobic material that provides sustained release properties to the compounds. As such, the compounds for use the method of the disclosure may be administered in the form of microparticles, for example, by injection or in the form of wafers or discs by implantation. In certain embodiments of the disclosure, the compounds of the disclosure are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours. The term pulsatile release is used herein in its conventional sense to refer to a drug formulation that provides release of the drug in such a way as to produce pulsed plasma profiles of the drug after drug administration. The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction and preparation conditions, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXAMPLES

The disclosure is now described with reference to the following Examples. These Examples are provided for the purpose of illustration only, and the disclosure is not limited to these Examples, but rather encompasses all variations that are evident as a result of the teachings provided herein.

### Methods and Materials

Unless specifically mentioned, expression of constructs in CHO cells or modified CHO cells with and without supplementation, enzymatic assays, AUC assay, half-life assay can be carried out using protocols described elsewhere herein or as known in the prior art.

### Area Under the Curve Assay

The area under the plasma concentration versus time curve, also called the area under the curve (AUC) can be used as a means of evaluating the volume of distribution (V), total elimination clearance (CL), and bioavailability (F) for extravascular drug delivery. Area under plasma time curve for each expressed and purified DNAse1-Fc and/or DNAse1L3-Fc construct can be carried out using the standard equation to determine half-life and bioavailability after a single subcutaneous injection of biologic, as described in Equation 1.

### Half-Life Determination

The drug half-life (t_{1/2}) is the time it takes for the plasma concentration or the amount of drug or biologic in the body to be reduced by 50%. Half-life values for each expressed and purified construct can be carried out following protocols described in the prior art and/or herein, such as Equation 1, which allows for determining half-life and bioavailability after a single subcutaneous injection of biologic.

Drug half-life can be calculated using Equation 1, which correlates the relationship between systemic fractional concentration and time of a drug administered to a subcutaneous depot in a single injection. Plotting the data as fraction of drug absorbed (F) over time (t) allows for the determination of the elimination (kₑ) and absorption (kₐ) constants by fitting the data to the equation for the total systemic absorption of a drug administered at a subcutaneous depot at time t=0.

$$F = \frac{{k}_{a}}{( {k}_{a} - {k}_{e} )} [ {e}^{- {k}_{e} t} - {e}^{- {k}_{a} t} ] \qquad ( Equation 1 )$$

### Additional Materials and Methods

Additional materials and methods used to generated the data and results of the examples are described.

Creation of a long acting, hyperactive, bioavailable enzyme therapeutic with dual DNASE1 and DNASE1L3 activity Mouse Dnase1 cDNA was amplified from a C57BL6/J cDNA library and cloned in-frame into the plasmid pFUSE-mIgG1-Fc1 (InvivoGen) to yield the parent DNASE1-Fc fusion protein, which consisted of 284 amino acids of the mouse DNASE1 fused by 7 amino acids to 222 amino acids of the CH2 and CH3 domains of mouse IgG heavy chain and hinge region. For the human version, cDNA for human DNASE1, codon optimized for CHO cell expression, was obtained from Integrated DNA Technologies, and cloned in frame into pFUSE-hIgG1-Fc1 (InvivoGen), Subsequent mutations were performed using QuikChange II XL Site Directed Mutagenesis (Agilent Technologies). All constructs were sequenced-verified before transfecting into CHO cells for protein production.

### DNASE1 and DNASE1L3 Activity Assays

DNASE1 activity was determined by reacting either 10 ml of conditioned CHO cell media, 10 ng of purified protein or 5 ml of plasma from a previously dosed mouse in a 20 ml solution containing 1 mg plasmid DNA, 100 mM Tris pH 7.5, 3 mM CaCl₂), 3 mM MgCl2, and 50 mM NaCl for 5-10 minutes at 37° C. and visualized on a 1% agarose gel. To analyze DNASE1L3 activity, nuclei were isolated from Wahi-3 cells (ATCC) using the Nuclei Isolation Kit (MilliporeSigma) and incubated in the same buffer in a final volume of 60 ml. After 30-45 minutes at 37° C., the DNA was extracted by adding 300 ml of 7M guanidine HCl, transferred to a Qiagen mini-prep spin column, washed, eluted, and run out on a 2.5% agarose gel. In some experiments, DNASE1 and DNASE1L3 activities were measured in mouse serum or urine without any buffer by adding plasmid DNA or purified Wehi-3 nuclei directly to serum.

Quantitation of cfDNA from Plasma or Urine by qPCR

To quantitate circulating cfDNA from mice, blood from a retro-orbital bleed in EDTA was centrifuged at 1,400×g for 10 minutes. The top layer was separated from the buffy coat, transferred to a new tube, and centrifuged at 14,000×g for 10 minutes, and the supernatant was transferred to a new tube. For qPCR we used 1 ml of platelet free plasma or 1 ml urine in a 20 ml solution of 1× SsoAdvanced Universal Inhibitor-Tolerant SYBR Green Supermix (BioRad) with the following DNA oligonucleotide sequences designed to hybridize to mouse retrotransposons; Sense 5′ CCTCTAGTGAGTGGAACACAACTTCTGC 3′ (SEQ ID NO: 62); and Anti-Sense, 5′ TGCAGGCAAGCTCTCTTCTTGC 3′ (SEQ ID NO: 63). Ct values are displayed in Prism GraphPad, and statistical significance calculated using a non-parametric Mann-Whitney T-test.

Digestion of Genomic and Mitochondrial cfDNA from Human SLE Whole Blood Using 1833

Whole blood in EDTA (with or without a 50 nM PMA pre-treatment for 2 hours at 37° C.) was supplemented with 20 mM each CaCl₂ and MgCl₂ before adding 200 nM 1833, incubating for 10 minutes at 37° C., centrifuged at 1000×g for 10 min at 4° C. to collect plasma for qPCR. In other experiments, plasma was first isolated from whole blood and then supplemented with 20 mM each CaCl₂ and MgCl2before adding 200 nM 1833 for 10 minutes at 37° C. For qPCR, 1 ul of the purified plasma was added directly into a 20 ul reaction using 1× SsoAdvanced Universal Inhibitor-Tolerant SYBR Green Supermix (BioRad) with DNA oligonucleotide sequences designed to hybridize to either human LINE1 5′ UTR (5′ CGAGATCAAACTGCAAGGCG (SEQ ID NO: 64) and 5′ CCGGCCGCTTTGTTTACCTA (SEQ ID NO: 65)) or the human mitochondrial tRNA-Phe gene (5′ CTAAATAGCCCACACGTTCCC (SEQ ID NO: 66) and 5′ AGAGCTCCCGTGAGTGGTTA (SEQ ID NO: 67)). Ct values are displayed in Prism GraphPad, and statistical significance calculated using a non-parametric Mann-Whitney T-test.

### Digestion of Human SLE Microparticle (MP) DNA by 1833

Whole blood (1 ml) from a human SLE patient, who was consistently positive for a high titer of anti-dsDNA Abs, was centrifuged at 1000×g for 10 min followed by the careful transfer of the top layer into a new tube. This procedure was performed twice to purposely avoid the transfer of any leukocytes from the buffy layer each time. The cell free plasma was then centrifugated 21,000×g for 1 hour at 4° C. After carefully aspirating the plasma, the MP pellet was washed with 1 ml PBS followed by another high-speed centrifugation and aspiration. The final MP pellet was resuspended in 100 ul PBS from which 10 ul was withdrawn and added to an equal volume of digestion buffer (200 mM Tris pH 7.5, 50 mM NaCl, 5 mM CaCl₂) and 5 mM MgCl2) in duplicate. One replicate received 1833 to a final concentration of 200 nM and the other an equivalent amount (1 ul) of PBS. The replicates were incubated at 37° C. for 10 min and the reaction stopped on ice. From the reaction, 1 ul was withdrawn and used in a qPCR reaction as described above.

Digestion of NETs from Human Leukocytes by 1833

Leukocytes were isolated by adding 10 ml of ACK buffer (MilliporeSigma) to 1 ml whole blood from either an SLE patient or an HC, for 5 min at RT to lyse RBCs, followed by centrifugation at 300×G for 5 min. The leukocyte pellet was washed with PBS and then plated into a poly-lysine coated optical bottom 96-well plate (Thermo Scientific), in RPMI without FBS and allowed to adhere for 30 minutes before adding PMA to a final concentration of 50 nM. After 2 hours at 37° C. to induce NETs, 50 nM 1833 was added for an additional 10-minute incubation at 37° C. and then paraformaldehyde was added to 2% and the plate removed to 4° C. for fixation. The next day, 5 mM Sytox Green (Thermo Scientific) was added, and NETs were visualized on a BZ-X Keyence fluorescence microscope with 488 nm laser light and emission collection at 449-552 nm.

### In Vivo Pharmacokinetics

DKO mice were injected s.c. with 1 mg/kg of the biologic, and both DNASE1 and DNASE1L3 activity were measured from serum at 2, 6, and 11 days after injection from at least 4 biological replicates per biologic.

### Genetic Model of Murine Lupus

The experimental results of both genders were considered separately and then combined, as similar findings were observed for both sexes. A total of 126 randomly assigned WT and DKO mice were injected s.c. weekly with either PBS or biologic at 1 mg/kg, starting at 2 weeks of age. Serum/plasma was collected via retro-orbital bleed from mice at 8, 14, 25, 40 and 52 weeks, and autoantibodies were measured by ELISA. At the 40-week bleed, all mice in the lupus study were i.p. injected with 500 ml pristane (MilliporeSigma). The surviving mice were euthanized at 52 weeks by cervical dislocation after isoflurane anesthesia. Kidneys, spleen, and lungs were removed, fixed in 10% neutral buffered formalin over-night, and paraffin-embedded for histological analysis. Spleens were weighed after over-night fixation before embedding. For immunofluorescence, unstained slides were deparaffinized, and heated in a pressure cooker for 5 min in 10 mM sodium citrate pH 6.0 for antigen retrieval. After blocking with 5% BSA in PBS containing 0.5% Tween-20 (PBS-T), slides were incubated with primary Abs, washed with PBS-T, and followed with Alexa Fluor conjugated secondary Abs #ab150121, #ab150120, #ab150068, and #ab96935 (Abeam). Primary antibodies used included Anti-MPO #AF3667, ST6GAL1 #AF5924 (R&D Systems) and Anti-Histone H3 (citrulline R2+R8+R17) #AB5103, and anti-C1q #AB155012 (Abeam).

### ELISA

Anti-dsDNA ELISAs were created using MaxiSorp 96 well plates (NUNC) pre-treated with 0.01% poly-Lysine in PBS for 2 hours at RT or over-night at 4° C., washed with PBS-T and coated with Calf Thymus DNA at 1 mg/well in PBS overnight at 4° C. Plates were washed 3 times and blocked with 5% BSA, 1% Normal Goat Serum PBS-T for 2 hours at RT or overnight at 4° C. After washing 3 times, plates were incubated with mouse serum/plasma at 1:300 dilution in 1% BSA PBS-T for 2 hours at RT or over-night at 4° C. Plates were washed 4 times with PBS-T and incubated with a 1:2000 dilution of goat anti-mouse IgG HRP-conjugated secondary antibody #ab97023 or IgM #ab97230 (Abcam) for 1 hour, washed again 4 times and incubated with 100 ml per well of 1-Step Ultra TMB (ThermoFisher Scientific) until desired color developed. The reaction was terminated with 2M sulfuric acid and absorbance measured at OD=450 nm on a Synergy Mx microplate reader (BioTek). The ELISA for analysis of anti-ssDNA autoantibodies was performed the same way, except the Calf Thymus DNA was first sonicated then heat denatured at 98° C. for 10 minutes and placed immediately on ice before plating out in poly-lysine 96-well plates at 4° C. as above. Anti-histone ELISA was performed similarly by binding 10 mg/ml histone from calf thymus (Roche Diagnostics) in 100 mM sodium carbonate, pH 9.4. For the detection of antinuclear antibodies (ANA), Wehi-3 cells (ATCC) were fixed with ice-cold methanol for 5 min, washed with PBS-T, and blocked with 5% BSA in PBS-T before incubating with a 1:300 dilution of mouse serum/plasma. For the 14-week samples the secondary Ab was a goat anti-mouse Alexa Fluor conjugate that was visually evaluated on a scale from 1-4 for signal intensity under a fluorescence microscope. The 25-week samples were prepared similarly but the evaluation was performed by measuring the fluorescence signal in each well using NIH ImageJ software. The 40- and 52-week ANA samples used a goat anti-mouse HRP secondary antibody followed by 1-Step Ultra TMB as above. Commercial ELISA assays were used for the following analytes: C—X—C Chemokine Motif Ligand 10 #ab100675), Creatinine #ab65340, C reactive protein #ab222511, EPO #ab270893, C3 #ab263884, Calprotectin #ab263885 and Surfactant-D #ab240683 (Abcam). Additional data for EPO, IL-6, CXCL9 and IL-11 was obtained from an EVE Technologies Mouse Cytokine/Chemokine 44-Plex Discovery Array. Antibodies against 1833 were detected by ELISA. Briefly, Nunc Maxisorp plates were coated with 200 ng of 1833. The plates were blocked for one hour with phosphate buffered saline plus 0.1% Tween-20 (PBST) with 3% non-fat milk. Serum/plasma was diluted 1:1000 in PBST 1% non-fat milk and assayed in duplicate using antigen-conjugated plates and plates without antigen for background subtraction. HRP conjugated goat anti-human IgG was used as a secondary antibody (Diluted at 1:10,000 in PBST 1% nonfat milk). Anti-1833 antibody arbitrary units (AU) were calculated using a standard curve made of a serial diluted serum from a high-titer SLE patient. The cutoff for anti-1833 positivity was defined as 2 SD above the mean of anti-1833 in healthy controls.

### Pristane Induced Lupus Model

10-14-week-old WT and DKO mice were injected i.p. with 500 ml pristane and weighed every 2-3 days until a 5% loss in body weight was detected, and then weighed every day thereafter. For the preventative study, DKO mice were randomly divided into two cohorts, and injected s.c. weekly with either 1 mg/kg biologic or PBS starting on the same day as pristane injection. In the therapeutic study, WT mice were randomly divided into two cohorts, and injected weekly with s.c. doses of either 1 mg/kg enzyme biologic or vehicle (PBS) beginning after the first mouse died, which was 9 days after pristane exposure for C57BL6/Tac mice, and 10 days after pristane exposure for C57BL/6Jax mice. Mice were euthanized and listed as non-survivors after the loss of greater than 25% body weight, or if the mice exhibited a significantly hunched posture or lethargic and labored breathing was detected. All non-surviving (euthanized) animals in the pristane trials exhibited visible pulmonary hemorrhage upon gross examination of the lungs and histologic evidence of alveolar damage upon microscopic examination of lung histology. Arterial oxygen saturation (SpO₂) readings were obtained using a MouseOx Plus Pulse Oximeter (STARR Life Sciences) on mice anesthetized under isoflurane.

### Statistical Methods

GraphPad Prism 10 was used to statistically analyze all data. Statistical significance between three or more groups was determined using an ANOVA comparison of means using the non-parametric Kruskal-Wallis independent test at a significance level of α=0.05. Comparisons between two groups were performed using a two-tailed Student's unpaired T-test. P values are denoted by the symbols *p<0.05, **p<0.01, ***p<0.001, unless explicitly indicated. Survival comparisons between treated and untreated mice in all animal models were determined using the Log Ranked (Mantel-Cox) test. Linear regression was performed with a simple linear regression model using Pearson r correlations and P values to determine significance.

Study approval: Animal procedures were approved by the Animal Care and Use Committee of Yale University (Animal Protocol #2022-11535) and complied with the US National Institutes of Health guide for the care and use of laboratory animals. Human studies were approved by the Hopkins Lupus Cohort under IRB NA_00039294 and NA_00001566 and by the Yale IRB under the Yale Lupus and Connective Tissue Disease Biorepository (HIC #1602017276). All patients consented in writing before inclusion.

### Analytical HPLC to Assess Biologic Purity

Protein sample (15 μg in PBS) was dried at 25° C. in SpeedVac for 20 minutes and reconstituted in 30 μL with 95% water, 5% acetonitrile, and 0.1% trifluoracetic acid. LC-PDA analysis was performed on a Waters H-Class UPLC system (Waters Technologies) utilizing a quaternary solvent system (Buffer A: 99.9% water, 0.1% trifluoracetic acid; Buffer B: 28.6% water, 71.4% acetonitrile, 0.075% trifluoracetic acid). Protein was profiled using an ACQUITY UPLC Protein BEH C4 Column 1.7 μm, 2.1 mm×150 mm (40° C.) and eluted at 0.4 mL/min with the following gradient: 28% buffer B at initial conditions; 100% B at 40 minutes; maintain 5 minutes; return to initial conditions at 50 minutes; and maintain for 20 minutes. The protein was detected in a PDA detector with 220 nm channel.

### Size Exclusion—Light Scattering Chromatography

The light scattering data were collected using a Superdex S-200, 10/30, HR Size Exclusion Chromatography column (GE Healthcare), connected to an Alliance 2965 HPLC system (Waters Corporation) equipped with an autosampler. The elution from SEC was monitored by a photodiode array (PDA) UV/VIS detector (996 PDA, Waters Corporation), differential refractometer (OPTI-Lab, or OPTI-rEx Wyatt Corporation), and static, multiangle laser light scattering detector (DAWN-EOS, Wyatt Corporation). The SEC-UV/LS/RI system was equilibrated in PBS at a flow rate of 0.5 ml/min. The weight average molecular mass, Mw, for the protein sample was tested at two concentrations, 0.59 mg/ml and 0.08 g/ml ( 1/20^{th} dilution). Two software packages were used for data collection and analysis. The Millennium software (Waters Corporation) controlled the HPLC operation and data collection from the multi-wavelength UV/VIS detector, while the ASTRA software (Wyatt Corporation) collected data from the refractive index detector, the light scattering detectors, and recorded the UV trace at 280 nm, 295 nm, or 310 nm sent from the PDA detector. 295 and 310 nm UV light were used for monitoring the elution of protein-DNA complexes, or for protein alone for when A₂₈₀>1.

### Generation of Dnase1/Dnase1L3-Double-Deficient (DKO) Mice

The Dnase1L3 cKO mouse model was generated via CRISPR-Cas9 methods. Potential Cas9 target guide (protospacer) sequences in introns 3 and 4 were screened using the online tool CRISPOR (http://crispor.tefor.net), and candidates were selected. Templates for sgRNA synthesis were generated by PCR, and sgRNAs were transcribed in vitro and purified (Megashortscript, MegaClear; ThermoFisher). sgRNA/Cas9 RNPs were complexed and tested for activity by zygote electroporation, incubation of embryos to blastocyst stage, and genotype scoring of indel creation at the target sites. The sgRNAs that demonstrated the highest activity were selected for creating the floxed allele. Guide RNA (gRNA) sequences are as follows: 5′ guide TCCAGAGCAGGCTCAAGTGG (SEQ ID NO: 68) and 3′ guide ACCTGACACAAATACCTTGG (SEQ ID NO: 69). Accordingly, a 550 base long single-stranded DNA (QssDNA) recombination template incorporating the 5′ and 3′ loxP sites was synthesized (Integrated DNA Technologies). The injection mix of sgRNA/Cas9 RNP+lssDNA was microinjected into the pronuclei of C57Bl/6J zygotes. Embryos were transferred to the oviducts of pseudo pregnant CD-1 foster females using standard techniques. Genotype screening of tissue biopsies from founder pups was performed by PCR amplification and Sanger sequencing to verify the floxed allele. Germline transmission of the correctly targeted allele (i.e., both loxP sites in cis) was confirmed by breeding and sequence analysis. Subsequently, positive mice were mated with a beta-actin-Cre mouse to generate constitutive Dnase1L3-deficient mice. The Dnase1-deficient mouse strain, C57BL/6NDnase1tm1.1(KOMP)Vlcg/TcpMuncd, RRID:MMRRC_047412-UCD was obtained from the Mutant Mouse Resource and Research Center (MMRRC) at the University of California, Davis. Dnase1-deficient mice were mated with Dnase1L3-deficient mice to generate the Dnase1/Dnase1L3-double deficient mice, referred to as DKO mice.

### Neutrophil Extracellular Trap (NET) Digestion Assay

Neutrophils were purified from mouse bone marrow as previously described. In brief, bone marrow was flushed from the mouse femur with a 26 g needle filled using RPMI supplemented with 10% FBS and 2 mM EDTA and passed through a 70 μM cell strainer, and cells were collected by centrifugation at 450×g for 7 minutes at 4° C. Cell pellet was resuspended in 20 mL of 0.2% NaCl for approximately 20 seconds, followed by addition of 20 ml of 1.6% NaCl and centrifugation at 450×g for 7 minutes at 4° C. After washing cells again in RPMI, the cells were resuspended in 1 mL PBS, layered on top of a Histopaque 1119/1077 density gradient, and spun at 872×g for 30 min without break. Neutrophils were collected from the interface, washed with RPMI without supplement, plated out into 96-well cell culture plates at 2.5×10¹ cells per well, and incubated at 37° C. in 5% CO2. NETs were induced by the addition of 50 nM PMA with or without a serial dilution from 25 nM to 0.325 nM of either Roche DNASE1, LBme (Construct 1687), or 1833. After 4 hours, 5 μM cell impermeable DNA binding Sytox Green (ThermoFisher Scientific) was added, and NETs were visualized on a BZ-X Keyence fluorescence microscope with 488 nm laser light and emission collection at 449-552 nm. The level of Sytox Green fluorescence was quantitated on a Synergy Mx microplate reader (BioTek).

Digestion of Genomic and Mitochondrial DNA in Human Whole Blood with LBme

Blood was isolated from lupus patients (LP) and healthy controls (HC) into BD microtainer EDTA tubes. 150 ul was removed and placed into duplicate PCR 8-tube strips (USA Scientific 1402-4700) and supplemented with CaCl₂) and MgCl2 at 20 mM to overcome the EDTA inhibition of DNASE. One 8-tube strip was spiked with 1.28 μM Lbme (Construct 1687) and serially diluted in whole blood on ice out to a final Lbme (Construct 1687) concentration of 10 nM, and the other was spiked with an equivalent volume of PBS as a control and serially diluted similarly. Both 8-tube strips were incubated at 37 C for 10 minutes and then placed on ice and centrifuged at 700×g for 10 minutes at 4° C. Plasma was transferred into new 8-well strips on ice. For qPCR, 1 μl of plasma was used in an 11 μl reaction using SsoAdvanced Universal Inhibitor-Tolerant SYBR Green Supermix (BioRad cat. 1725017) following the manufacturer's instructions. Amplification of genomic DNA was performed using oligos designed against a human Line1 5′UTR region (5′ CGAGATCAAACTGCAAGGCG 3′ (SEQ ID NO: 70) and 5′ CCGGCCGCTTTGTTTACCTA 3′ (SEQ ID NO: 71)) and using oligos designed for the amplification of mitochondrial DNA (5′ CTGTTCCCCAACCTTTTCCT 3′ (SEQ ID NO: 72) and 5′ CCATGATTGTGAGGGGTAGG 3′ (SEQ ID NO: 73)).

1833 Digestion of Genomic and Mitochondrial cfDNA from PMA Treated Whole Blood

The whole blood from two healthy controls (HC) and an SLE patient in EDTA was stimulated with 50 nM PMA for 2 hours at 37° C. to induce NETosis. 20 mM CaCl₂) and 20 mM MgCl2 was added and spiked with either 1833 to 100 nM or with an equivalent volume of PBS and incubated another 10 minutes at 37° C. The whole blood was then centrifuged at 1000×g for 10 minutes at 4° C. and 1 μl of the plasma was used in a qPCR reaction.

### EXAMPLES

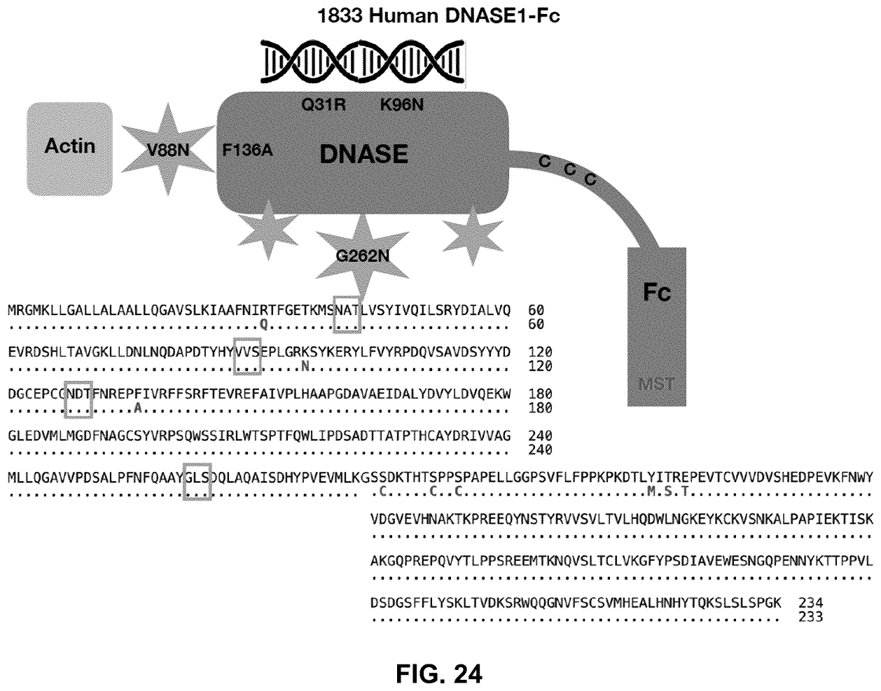

FIG. 24 illustrates a non-limiting DNAse1-Fc construct of the disclosure (Construct 1833), with certain contemplated point mutations highlighted.

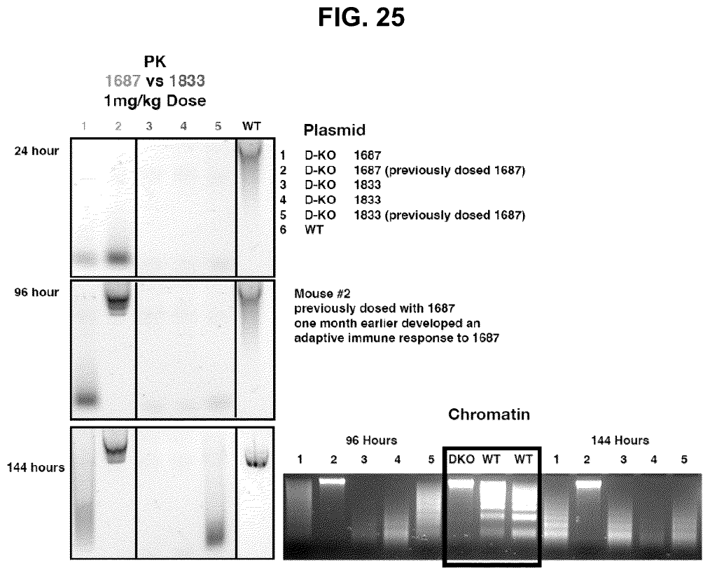

FIG. 25 illustrates the finding that Construct 1833 effectively promoted NET degradation in a mouse model (DNAse1 and DNAse1L3 double knock-out mice or DKO). Construct 1833 was administered at the same dose as Construct 1687 and resulted in more complete NET degradation than Construct 1687 (see mouse 1 vs. mouse 3 or 4). Interestingly, mouse 2 had been previously dosed with Construct 1687 and found to have developed an immune response to Construct 1687; subsequence administration of mouse 2 with Construct 1833 showed much improved NET degradation.

The present studies use an established animal model for diffuse alveolar hemorrhage (DAH), which is based on induction of Dah in C57/BL6 mice using pristane (also known as 2,6,10,14-tetramethylpentadecane). This model has been described in Jarrot, et al., 2019, J. Autoimmunity, 100:120-130; Smith, et al., 2018, JCI Insight, 3(15):e120798; and Tumurkhuu, et al., 2022, Front. Immunol. 13:790043.

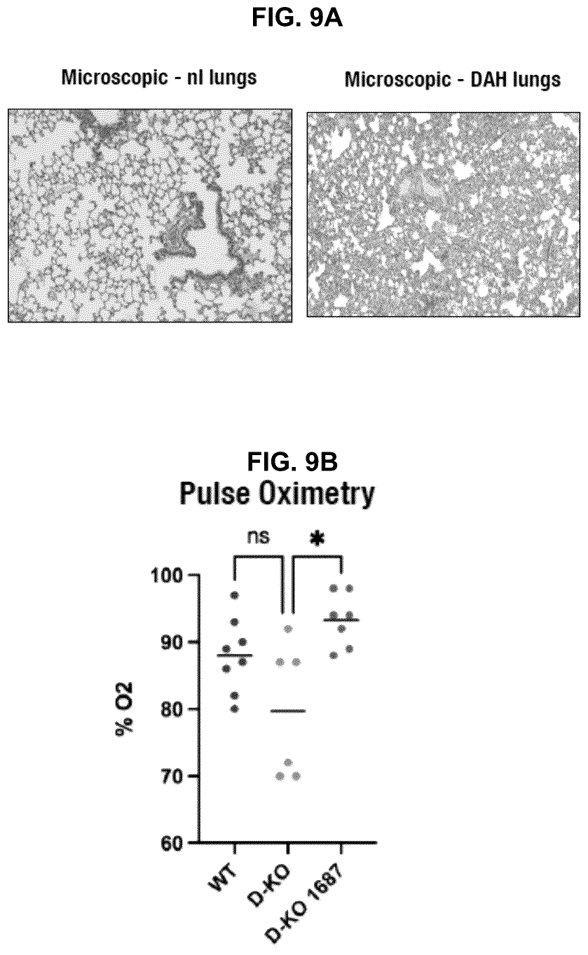

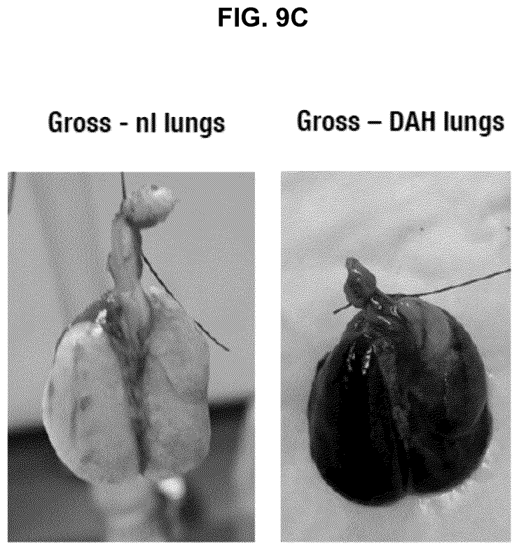

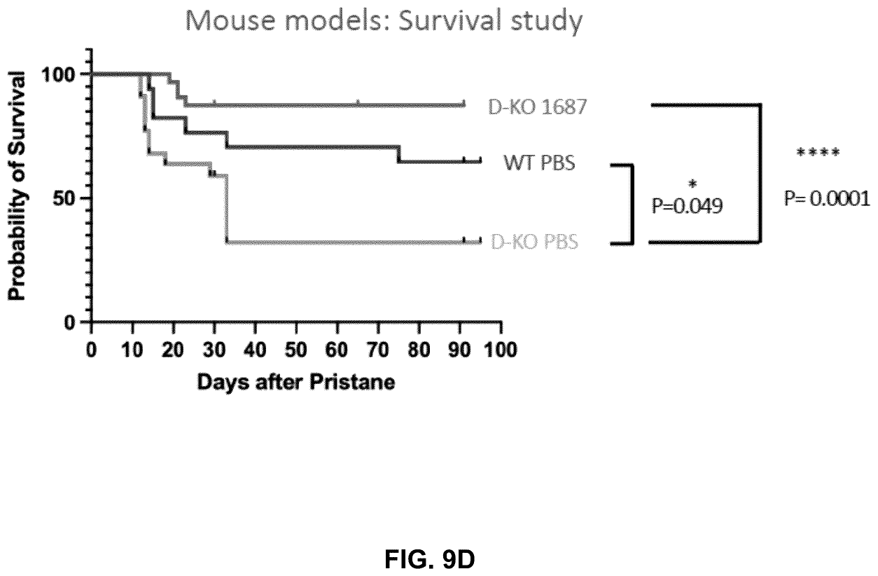

FIG. 9A illustrates microscopic images of lung tissues for normal lungs and DAH lungs. FIG. 9B illustrates % oxygen measurements for WT mice, DNAse1 and DNAse1L3 double knock out (DKO) afflicted with DAH, and DKO mice afflicted with DAH and treated with a construct of the disclosure. FIG. 9C illustrates lung gross histology of normal lungs and DAH lungs. FIG. 9D illustrates a survival study for DKO (top and bottom lines) and WT (middle line) mice afflicted with DAH (induced by pristane administration at day 0) and DKO treated with a construct of the disclosure on day 0 (top line).

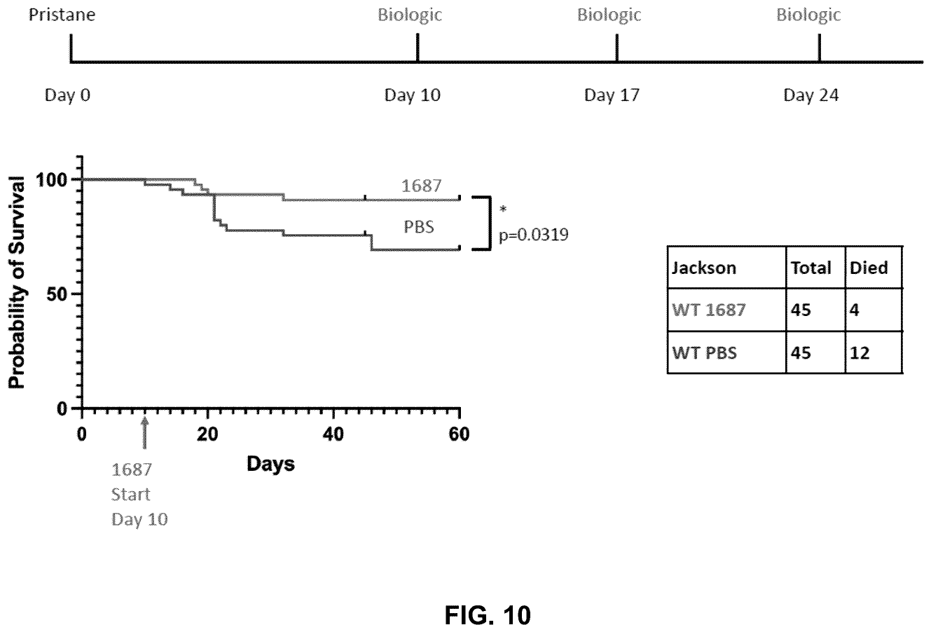

FIG. 10 illustrates a survival study for WT C57BL/6J mice (C57BL6 mice obtained from Jackson Labs) afflicted with DAH (induced by pristane administration at day 0) and treated with a construct of the disclosure starting at day 10 (and then at day 17 and day 24). The construct-treated mice showed statistically significant higher survival rates than the untreated mice. The results demonstrate that WT mice, and by extension genotypically normal humans without monogenic disorders in DNAse genes, can benefit from a DNA degrading biological agent, such as the constructs of the disclosure, during pathologies associated with aberrant NETosis pathologies such as DAH.

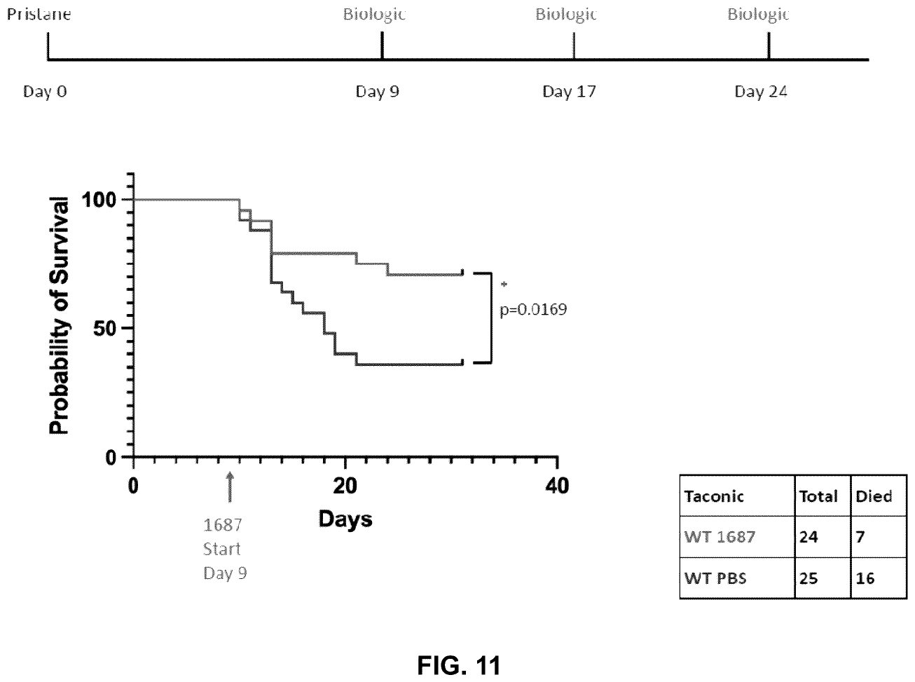

FIG. 11 illustrates a survival study for WT C57BL/6J mice (C57BL6 mice obtained from Taconic Labs) afflicted with DAH (induced by pristane administration at day 0) and treated with a construct of the disclosure starting at day 9 (after the first animal dies), and then day 17 and day 24. The construct-treated mice showed statistically significant higher survival rates than the untreated mice.

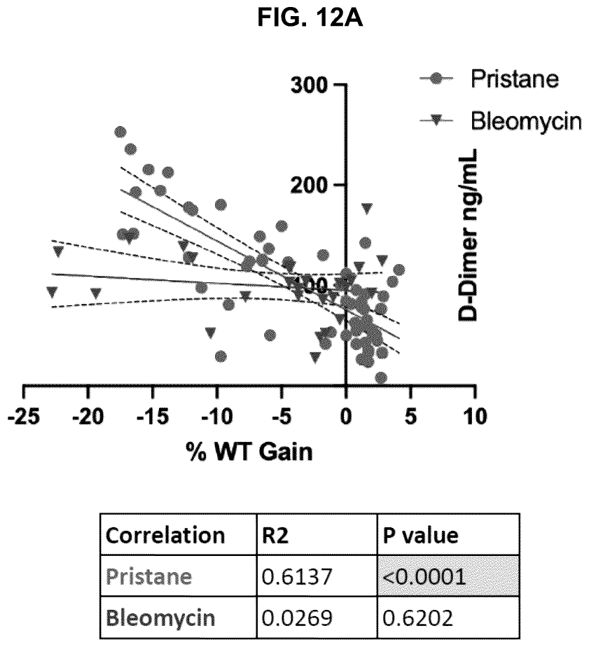

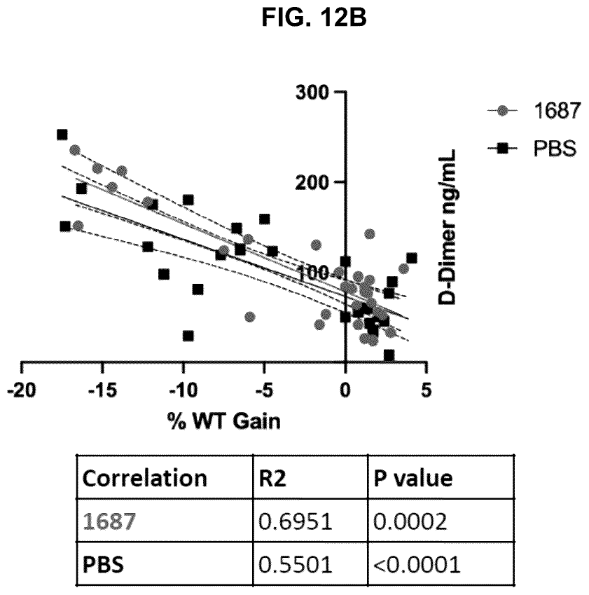

FIGS. 12A-12B illustrate measurements of D-dimer (which is a fibrin degradation product) at day 12 in DAH-induced mice (red symbols—Pristane). Coagulopathies such as disseminated intravascular coagulopathy are know to induce morbidity and mortality in DAH patients. Elevated D-dimers are predictive biomarkers for disseminated intravascular coagulation (DIC) and COVID-19 coagulopathies. The data indicate that DIC is present in the pristane-induced murine model of DAH and correlates directly with weight loss and death, simulating the coagulopathies experienced in humans suffering from DAH.

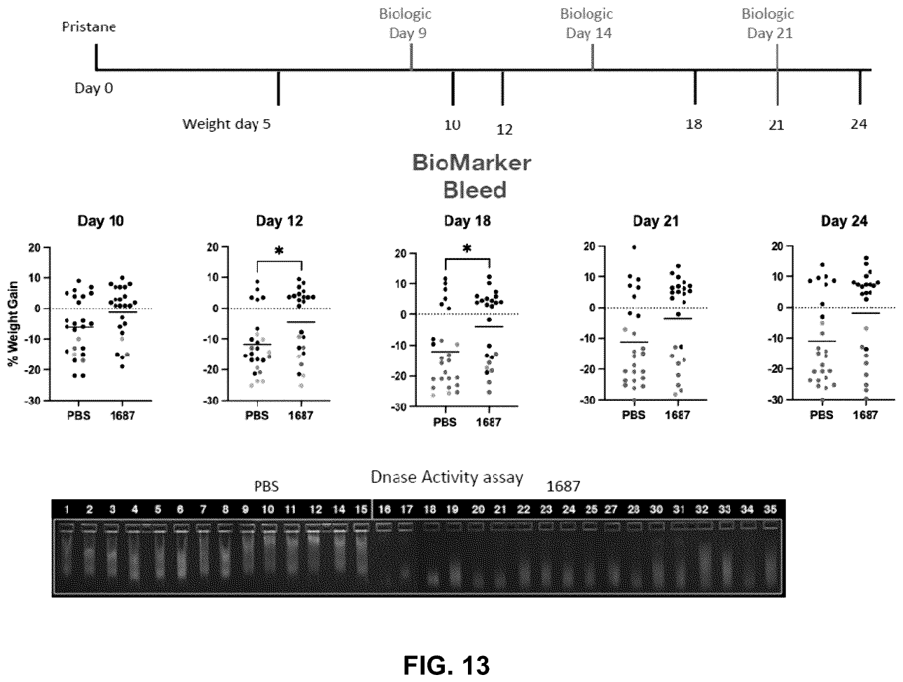

FIG. 13 illustrates a survival study for WT C57BL/6NTac mice afflicted with DAH (induced by pristane administration at day 0) and treated with a construct of the disclosure starting at day 9 (and then at day 14 and day 21). Weights of the mice were measured on days 5, 10, 12, 18, 21, and 24. The data indicate both treated and untreated DAH mice suffer significant weight loss, but significantly more mice treated with the subject of the invention rescues are rescued from death (FIG. 11), demonstrating that the constructs of the invention provide significant benefit even to critically ill animals. Mice that died are represented by red symbols (dark grey). Mice that survived are represented by cyan symbols (light grey)

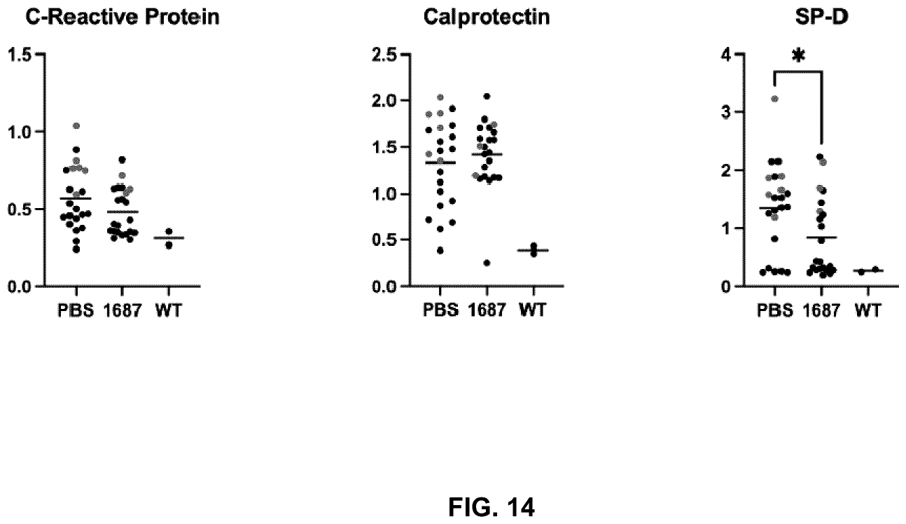

FIG. 14 illustrates measurements of C-reactive protein, calprotectin, and SP-D in WT mice with DAH induced by pristane that are treated with vehicle (PBS) or the construct of the disclosure (Construct 1687). Calprotectin, a neutrophil cytosolic protein, was elevated in both treated and untreated mice demonstrating equal NETosis in both groups. However, Surfactant Protein D, a marker of pulmonary permeability, was significantly less in the mice dosed with the construct of the disclosure (Construct1687). Concentrations of C-reactive protein rise in response to inflammation. It is an acute-phase protein of hepatic origin, which increases following interleukin-6 secretion by macrophages and T cells. C-reactive protein's physiological role is to bind to lysophosphatidylcholine expressed on the surface of dead or dying cells (and some types of bacteria) to activate the complement system via C1q. Calprotection comprises as much as 60% of the soluble protein content of the cytosol of a neutrophil, and is secreted by an unknown mechanism during inflammation, with antimicrobial, proinflammatory and prothrombotic properties. Surfactant protein D is a lung surfactant protein part of the collagenous family of proteins called collectin. Calprotectin was elevated in both cohorts indicating an equal amount of NETotic events. However, Surfactant Protein D, a marker of pulmonary permeability, was significantly less in the mice dosed with the construct of the disclosure. Mice that died are in red (dark grey).

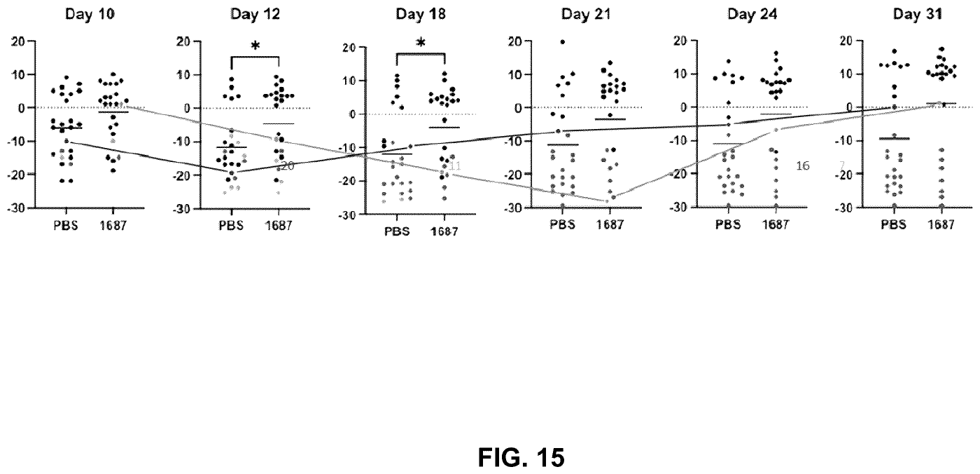

FIG. 15 illustrates weight loss for DAH mice treated with a construct of the disclosure in which individual animals which drop significant weight are followed throughout the 31 day experiment. A surviving vehicle treated mouse is followed with Line A, and a surviving treated mice is followed with Line B. In general, mice dosed with the construct of the invention showed significantly less weight loss at days 12 and 18 as compared to untreated DAH mice. Four mice of each cohort dropped weight but fully recovered by day 31. Four mice of each cohort dropped weight but fully recovered by day 31. The data demonstrate that the constructs of the invention rescue mortality even in critically ill animals that drop significant body weight, as demonstrated by the 30% weight loss exhibited by the treated animal that survived pristane induce DAH mortality (Line B). In comparison the most severe weight loss exhibited by a surviving vehicle treated animal was 20% (Line A).

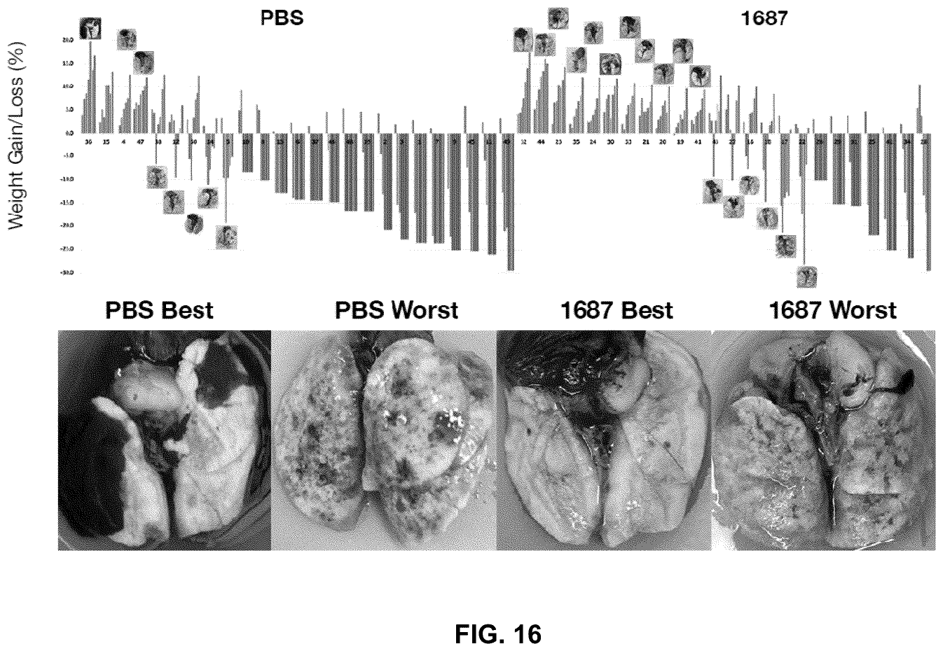

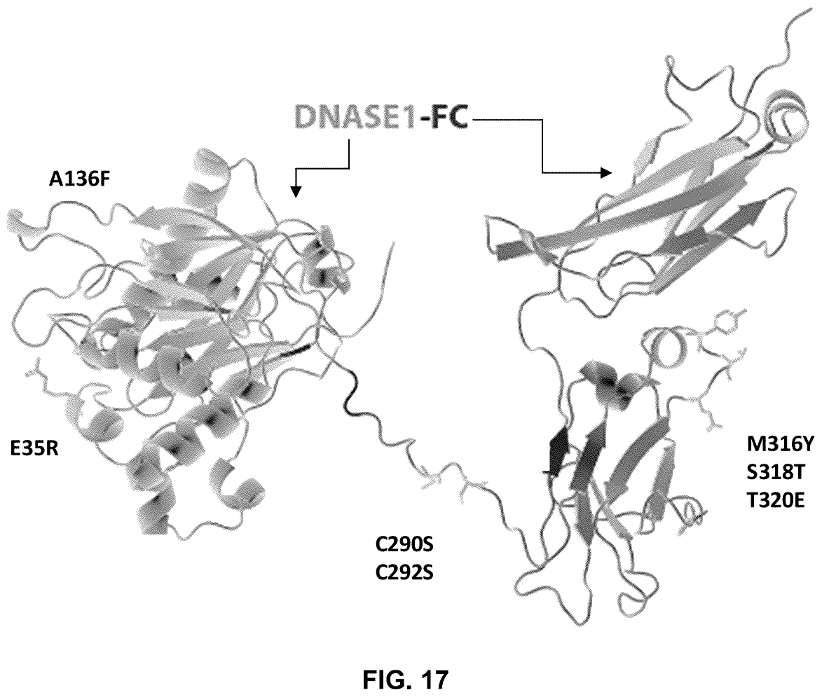

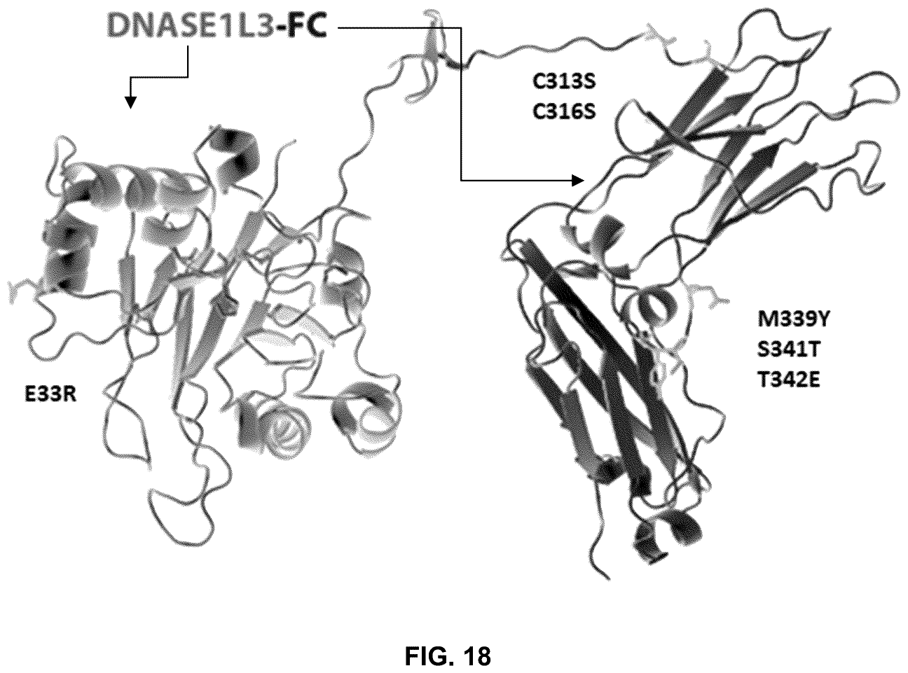

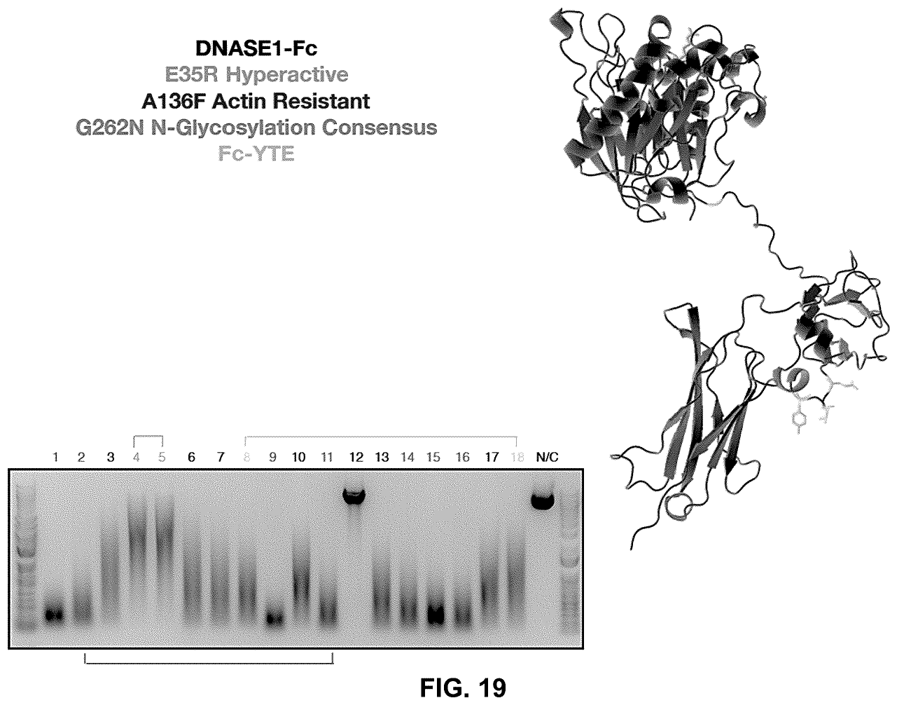

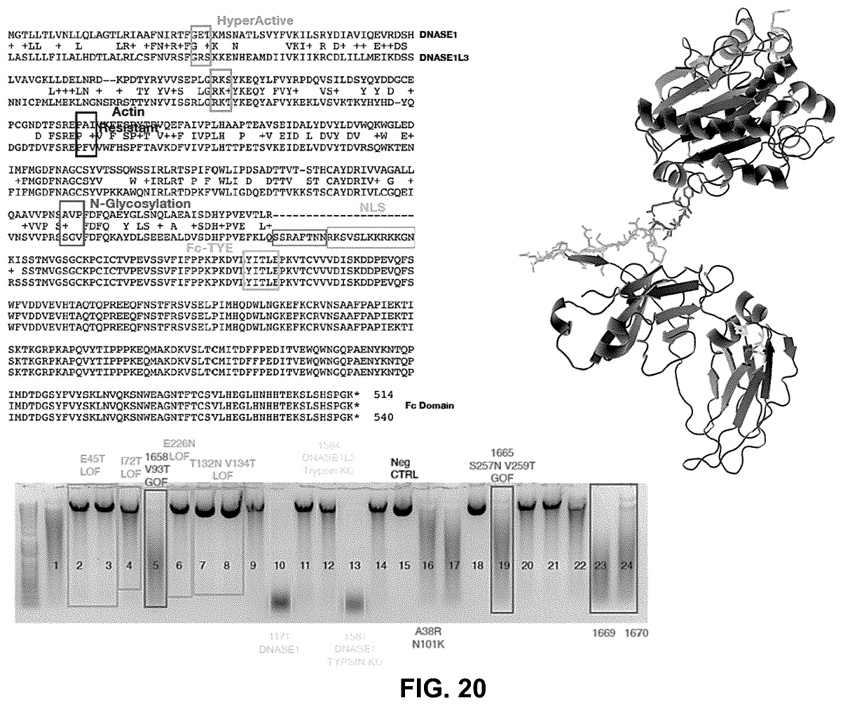

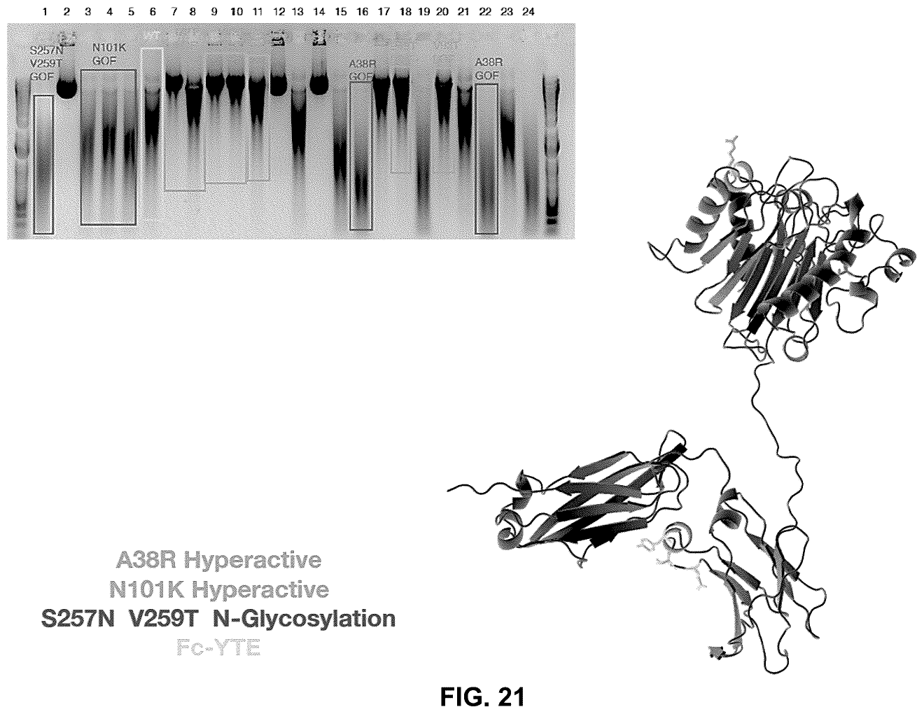

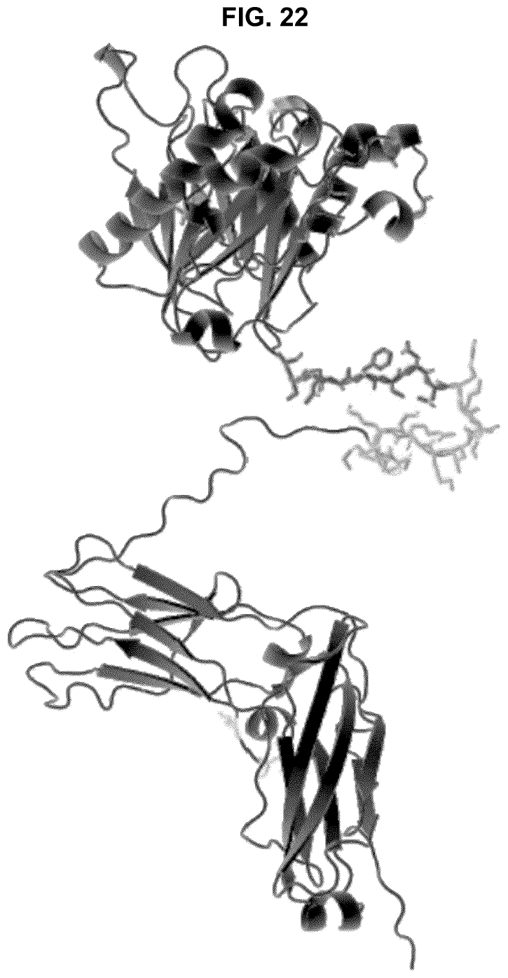

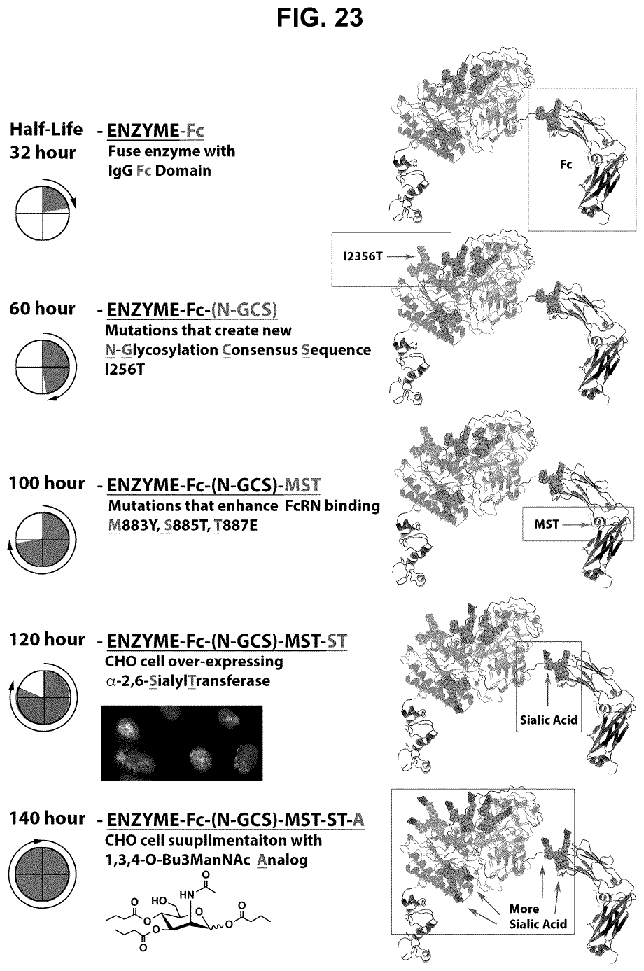

FIG. 16 illustrates waterfall plots illustrating the weight loss in mice after pristane-induced DAH, with mortality labelled in red/dark grey bars). Shown also is selected lung gross histology representing least effected (Best) and most effected (Worst) treated (Construct 1687) and untreated (PBS) mice. The data demonstrates that significantly greater number of vehicle treated animals (labelled ‘PBS’) suffered severe weight loss than threated animals (labelled ‘1687’).

The data provided herein show complete suppression of auto-immune antibodies for 9 months with weekly doses of a construct of the disclosure. The constructs of the disclosure are non-toxic, non-immunogenic, long acting, bioavailable, and effective against genetic models of autoimmunity. As demonstrated herein, the constructs of the disclosure prevent death from DAH in affected animals. An illustrative construct of the disclosure improved survival in the accepted DAH animal model when dosed in a therapeutic mode (10 days after provocation with pristane). It should be noted that the animal model used herein exhibits the coagulopathy also present in human DAH. Further, the blood biomarkers monitored herein (D-surfactant protein, Calprotectin) are predictive of disease mechanism and survival.

### FURTHER EXAMPLES

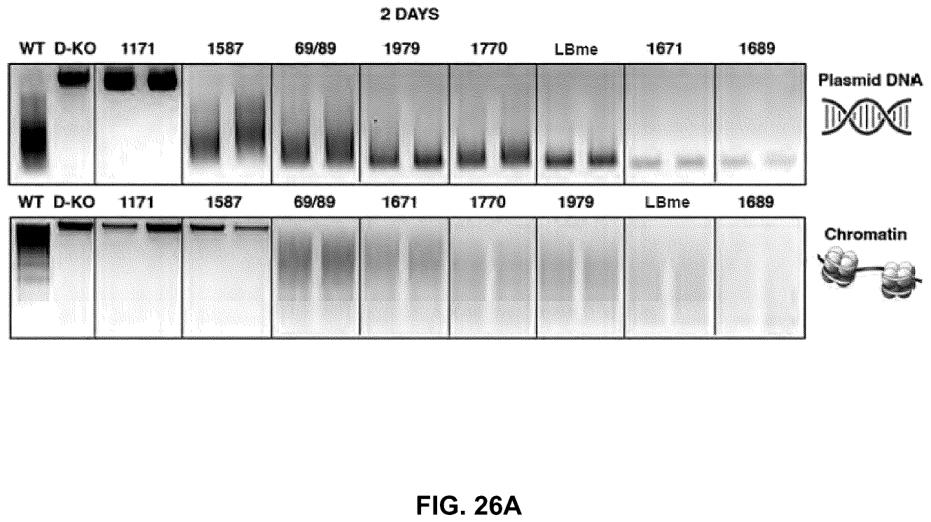

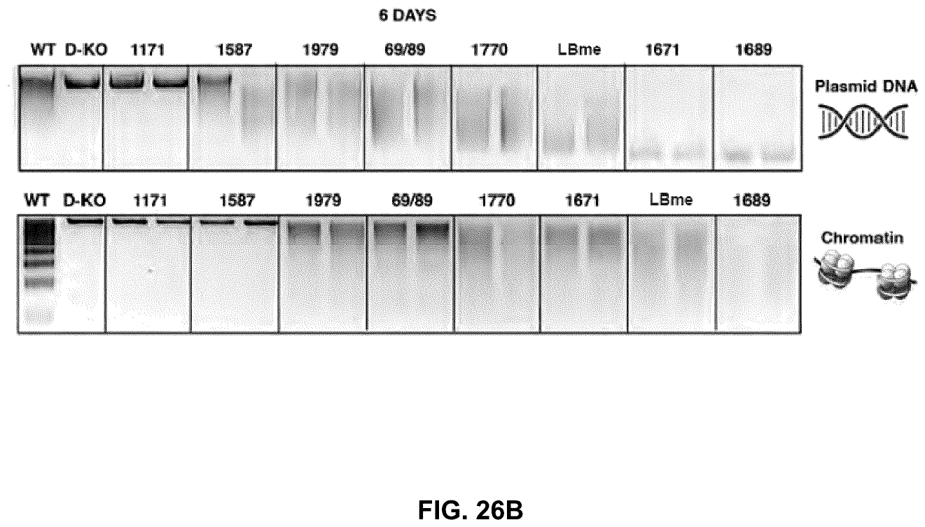

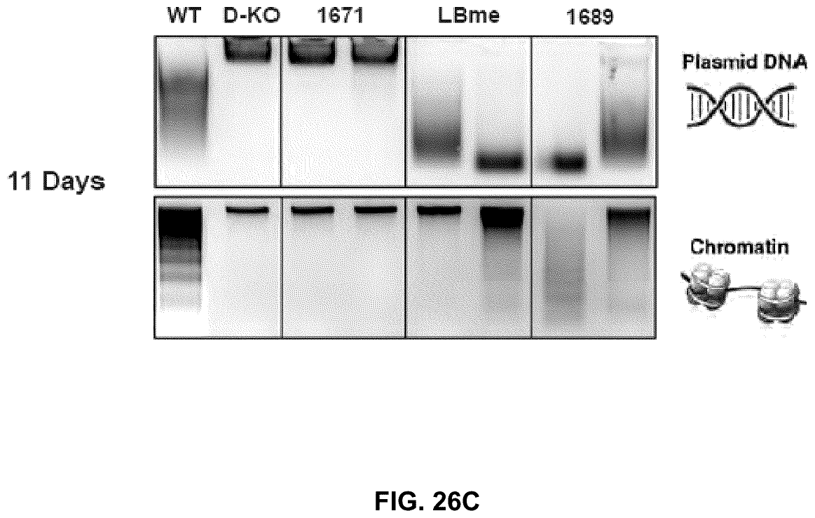

FIG. 26A-FIG. 26C illustrate the pharmacodynamic activity of various purified DNASE1 isoforms evaluated in vivo by dosing DKO mice with a single s.c. injection of each biologic at 1 mg/kg and withdrawing blood from the mice at two (FIG. 26A), six (FIG. 26B), and 11 days (FIG. 26C) following dosing. Plasma was isolated from the blood samples and exogenous free DNA and chromatin was added. The samples were then incubated at 37° C. for five minutes, and run on agarose gels to image degradation of the exogenous DNA (or lack thereof) of each isoform at various time points. Most biologics exhibited full PD activity 2 days after dosing, and three isoforms—1671, 1689, and 1687—exhibited full PD activity 6 days after dosing. Plasma from these three mice was drawn 11 days after dosing, revealing the murine isoforms with the longest PD activity to be 1687 and 1689.

FIG. 27 illustrates the prevention of autoimmunity in DKO mice by 1687. WT mice treated with weekly injections of PBS, and DKO mice treated with weekly s.c. doses of PBS (vehicle) or 1687 at 1 mg/Kg beginning on the second week of life. Plasma samples were taken at 8, 14, 25, and 40 weeks and titers of anti-nuclear, anti-histone, and anti-dsDNA autoantibodies were evaluated with by ELISA. In comparison to WT controls, by 8 weeks of age the PBS treated DKO mice (DKO) spontaneously elevated anti-dsDNA, anti-histone, and anti-nuclear antibodies, whereas 1687 treated DKO mice (1687) did not elevate auto-antibodies suggestive of lupus. At 40 weeks all mice were challenged with pristane (500 ul IP), and animals were followed over the next 12 weeks. At 52 weeks the ANAs in the surviving DKO mice remained elevated, however anti-dsDNA abs were no longer elevated, demonstrating increased mortality in vehicle treated DKO mice with elevated anti-dsDNA antibody titers. *p<0.05, **p<0.01, ***p<0.001, ****p<0.0001, ANOVA Kruskal-Wallis test.

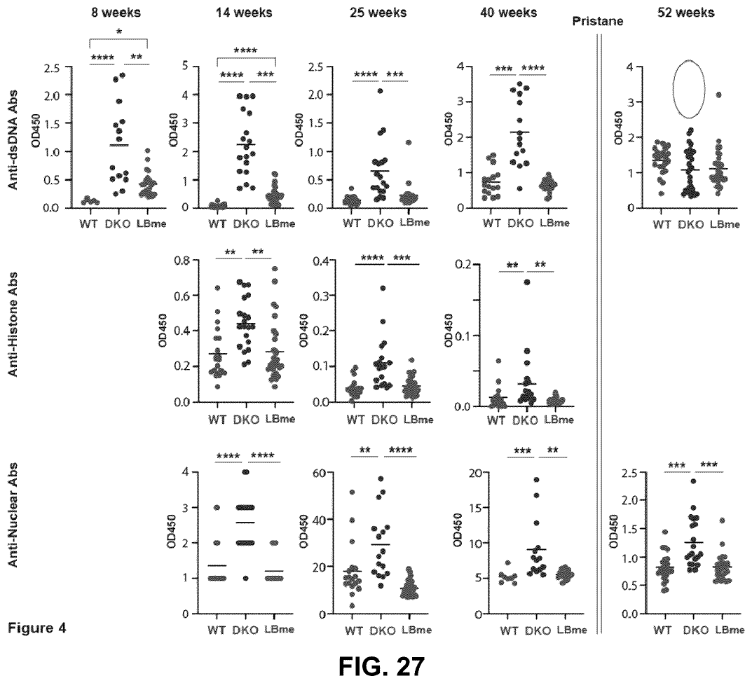

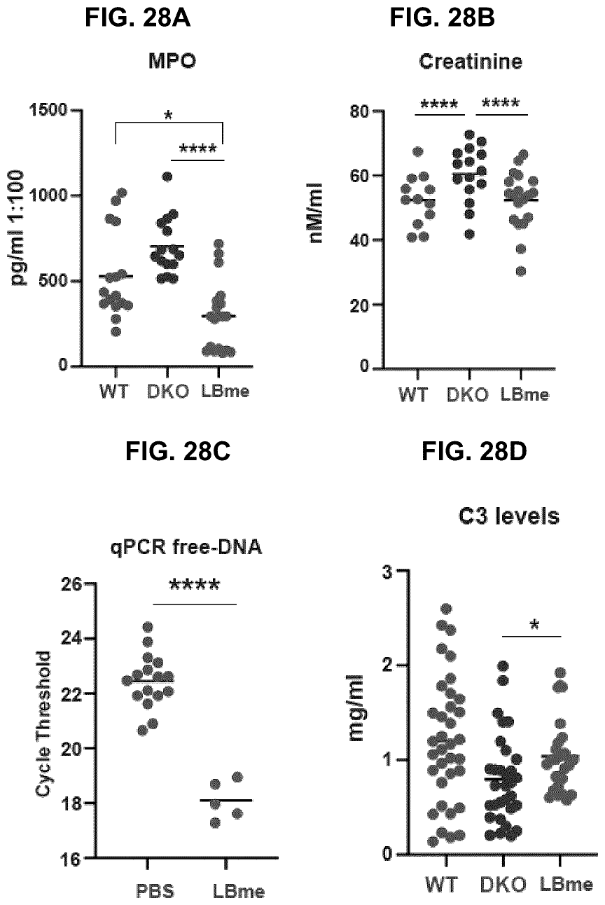

FIG. 28A-FIG. 28D illustrate the plasma biomarkers of DKO mice following pristane challenge. FIG. 28A presents serum anti-MPO antibodies in pristane treated lupus mice 4-weeks after pristane treatment (at 44 weeks). FIG. 28B presents creatinine levels in pristane treated lupus mice 4-weeks after pristane treatment (at 44 weeks). FIG. 28C presents quantitative PCR of free DNA in pristane treated lupus mice 4-weeks after pristane treatment (at 44 weeks). *p<0.05, **p<0.01, ***p<0.001, ****p<0.0001, ANOVA Kruskal-Wallis test (FIG. 28A-FIG. 28C). FIG. 28D presents C3 complement levels in the lupus mice at 52 weeks. *p<0.05 Students two tailed T-test, vehicle (DKO) and 1687 treated (1687) DKO mice.

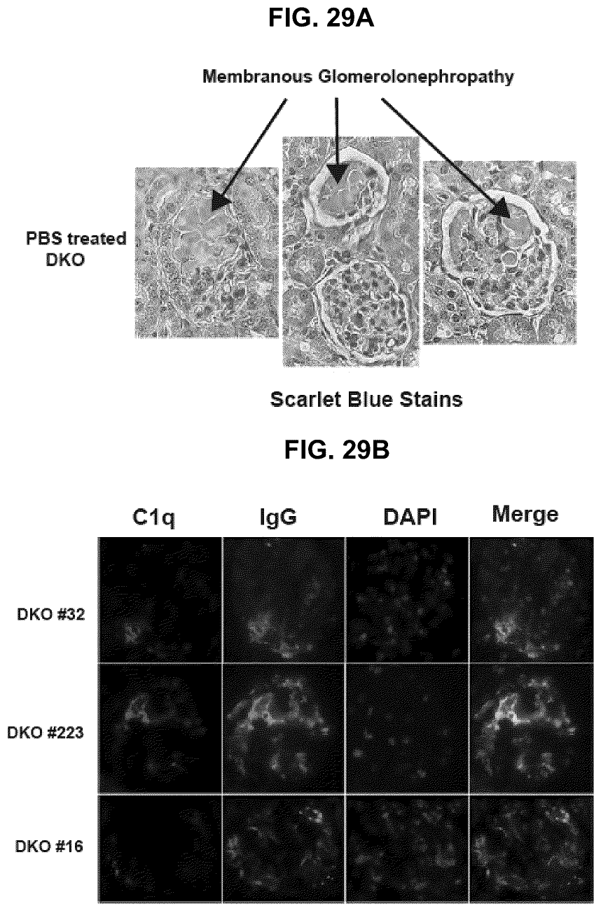

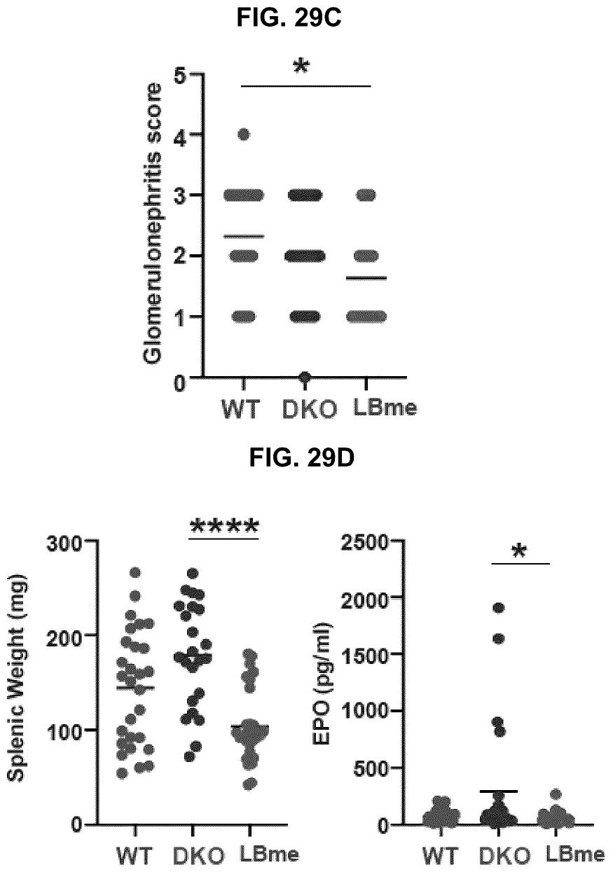

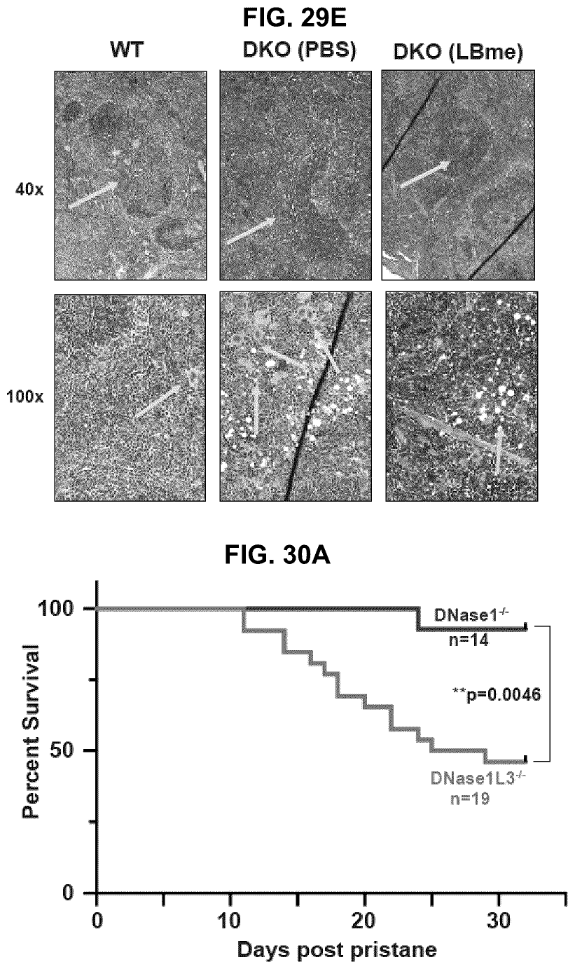

FIG. 29A-FIG. 29E illustrate membranous glomerulonephropathy and immune complex deposition in glomeruli of vehicle treated DKO mice. FIG. 29A presents light microscopic exam of the kidneys of untreated DKO mice that revealed the presence of membranous glomerulopathy in some untreated DKO mice (Martius Scarlet Blue stains). These findings were not present in the 1687 treated DKO cohort. FIG. 29B presents examination of affected kidneys in the untreated DKO mice that revealed evidence of revealed immunocomplex deposition via immunofluorescence staining with IgG and C1q. FIG. 29C demonstrates that glomerulonephritis assessed in a blinded fashion by a board-certified nephropathologist revealed a lower glomerulonephritis score in 1687 treated DKO mice than in WT controls, but no significant differences in the treated and untreated DKO mice. FIG. 29D demonstrates that spelnomegaly was significantly present in vehicle treated DKO mice in comparison to their 1687 treated siblings, as was erythropoietin (EPO) at 52 weeks. FIG. 29E demonstrates that histologic examination of the spleens revealed white pulp expansion due to coalescence of lymphoid follicles in vehicle treated DKO mice (yellow arrows). Vehicle treated DKO mice also exhibited robust extramedullary hematopoiesis in comparison to 1687 treated siblings and WT controls (cyan arrows). *p<0.05, **p<0.01, ***p<0.001, ****p<0.0001, ANOVA Kruskal-Wallis test.

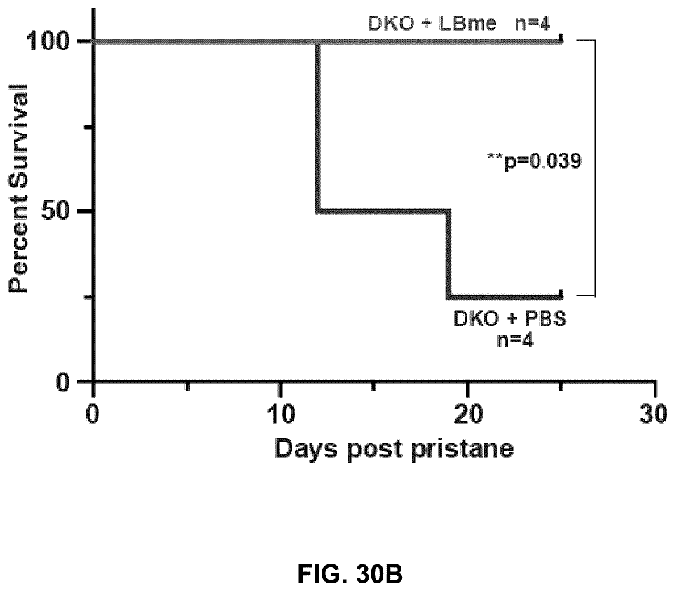

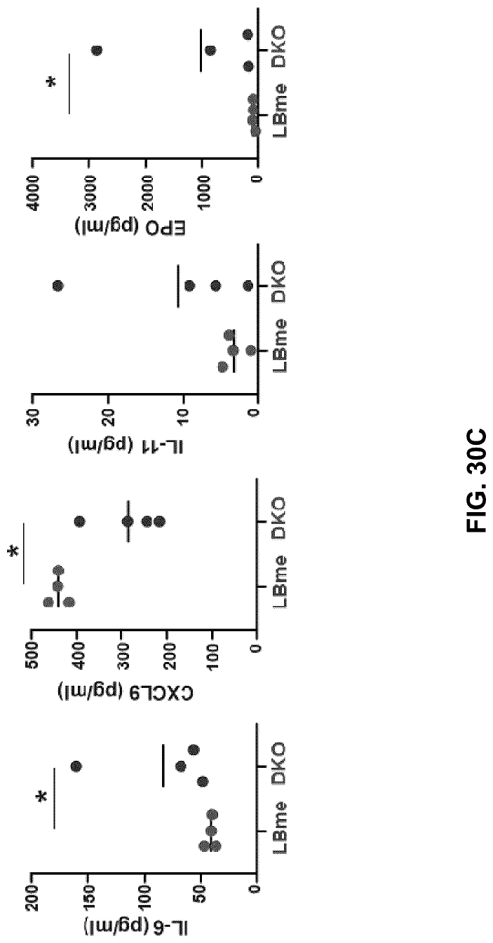

FIG. 30A-FIG. 30C present survival and plasma biomarkers of mice as follows. FIG. 30A demonstrates that a survival comparison of DNAse1^{-/-} and DNAse1L3^{-/-} mice on C57BL/6J backgrounds following pristane challenge revealed increased mortality in DNAse1L3^{-/-} mice compared to DNAse1−/− counterparts (50% vs less than 10%, respectively, p=0.0046. Mantel-Cox), illustrating the effect of functional loss of DNAse1L3 activity on the acute autoinflammatory phenotype. FIG. 30B demonstrates that the mortality induced by pristane challenge in DKO mice could be prevented with weekly 1 mg/Kg s.c doses of 1687 (100% survival in dosed vs. 25% survival in vehicle treated DKO mice, p=0.039, Mantel-Cox). FIG. 30C demonstrates that the plasma biomarkers in the dosed and undosed DKO mice revealed higher levels of IL6 and erythropoietin (EPO) in the undosed animals, and lower levels of CXCL-9. IL-11 trended higher without significance in this limited (n=4) study. *p<0.05, two tailed Student's unpaired T-test.

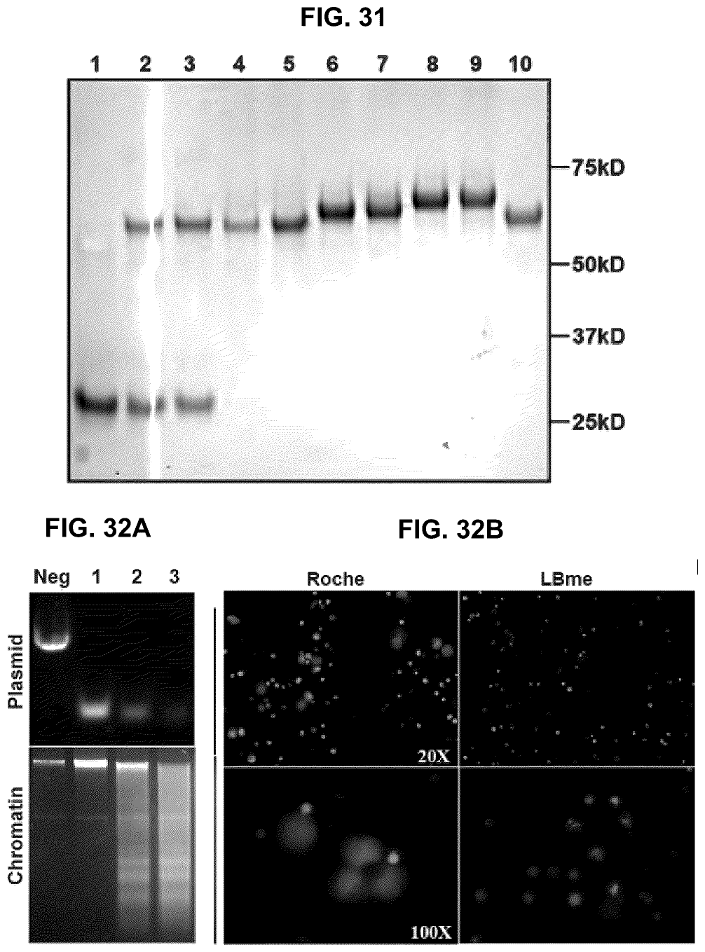

FIG. 31 presents a Coomassie stained gel of some of the CHO cell purified proteins used in the present studies. Lanes 1, 2, and 3 show three unsuccessful attempts to purify full-length mouse DNASE1-IgG1-Fc fusion protein (predicted MW of 58 kD) from construct 1171. A cryptic trypsin cleavage site, identified and mutated by a single R290G amino acid substitution, resulted in a well-behaved full-length protein (Lane 4 construct 1585). Lane 5, construct 1671 comprises a hyperactive E35R amino acid substitution. Lanes 6 and 7, show two preparations of construct 1687 with a mobility shift indicative of the successful N-linked glycosylation of the newly engineered glycosylation consensus sites. Similarly, lanes 8 and 9 are two preparations of construct 1689 also glycosylation mobility shift. Lane 10 is human DNASE1-IgG1-Fc, construct 1833.

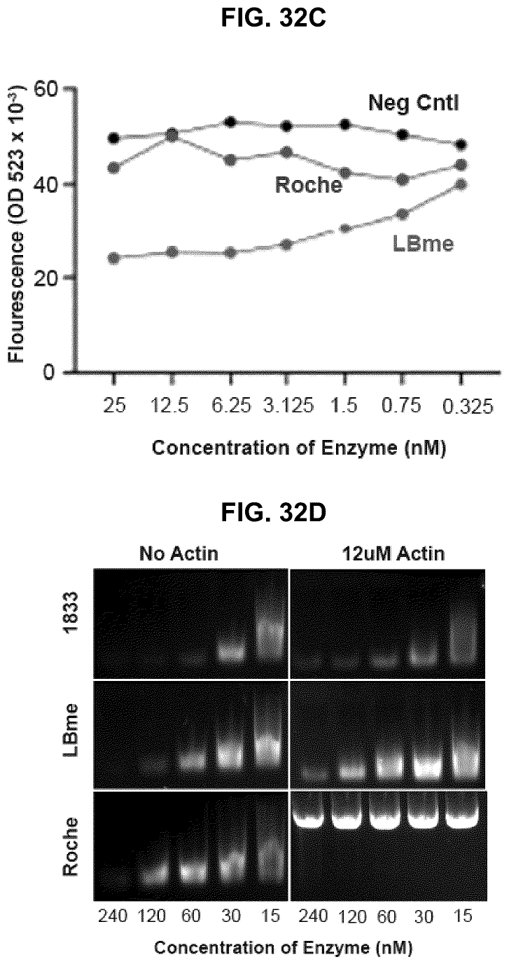

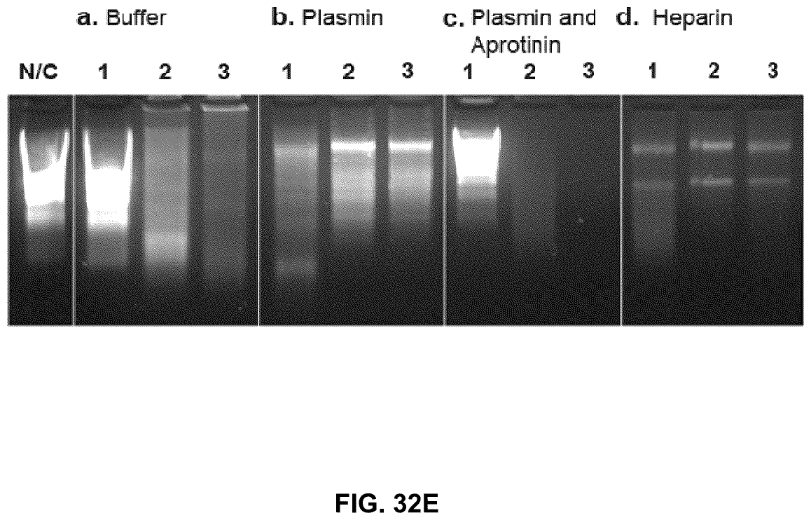

FIG. 32A-FIG. 32E present data and results related to biochemical characterization of dual activity biologics. FIG. 32A demonstrates that commercial DNASE1 (lane 1, Roche) digests plasmid (top) but not chromatin DNA (bottom), in contrast to 1687 (lane 2) or 1833 (human isoform of 1687, lane 3). Neg—negative control (plasmid DNA only). FIG. 32B demonstrates that 1687 degrades NETs in a concentration dependent manner, in contrast to DNAse1. Quantitation of NET degradation was measured using SYTOX green fluorescent labelling. FIG. 32C demonstrates that 1687 degrades NETs in a concentration dependent manner, in contrast to DNAse1. Quantitation of NET degradation was measured using SYTOX green fluorescent labelling. FIG. 32D demonstrates that 1687 and human analog (1833) are not inhibited by Actin, in contrast to commercial DNASE1 (Roche). FIG. 32E demonstrates the effects of Plasmin, Aprotinin, and Heparin on commercial DNASE1 (lane 1, Roche), 1687 (lane 2), and 1833 (lane 3). a. Histone bound DNA in buffer alone (N/C), and in the presence of DNASE1 (1), 1687 (2), and 1833 (3). Only 1687 and 1883 hydrolyze histone bound DNA. b. Plasmin activates DNASE1 inter-nucleosome chromatin cleavage. c. Plasmin activation of chromatin cleavage by DNAse1 is inhibited by Aprotinin (lane 1), but has no effect on 1687 or 1833 (lanes 2 and 3). b & d. Plasmin and heparin, known inhibitors of DNASE1L3 [49], have no effect on 1687 or 1883 cleavage of Chromatin. The bands present in d are the 18S and 28S Ribosomal subunits which are digested by endogenous RNAse in serum.

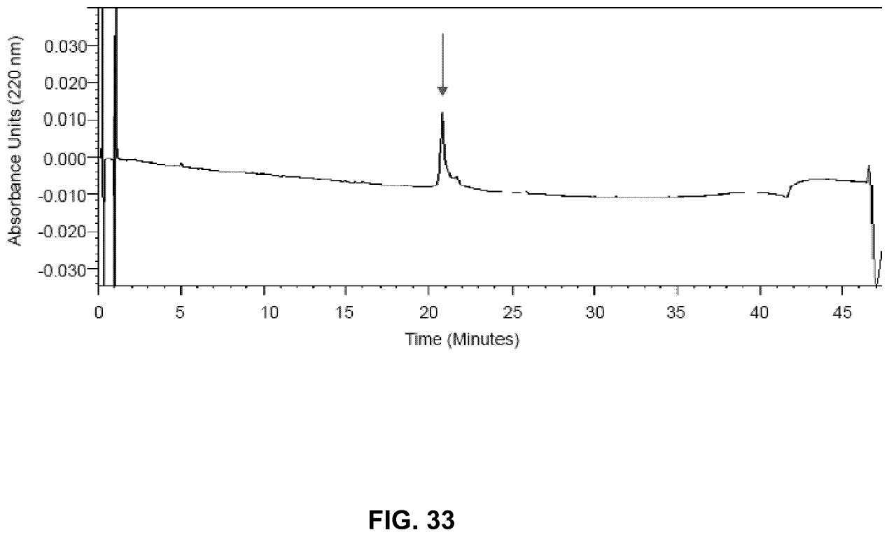

FIG. 33 presents chromatographic profiling of Construct 1833. Reverse phase HPLC with a protein BEH C-4 column eluted Construct 1833 as a single peak with a small trailing peak in approximately 46% acetonitrile.

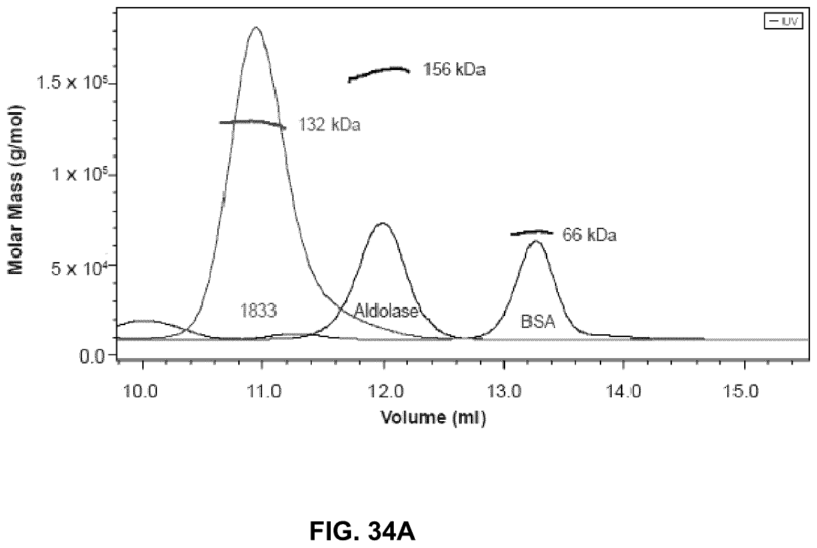

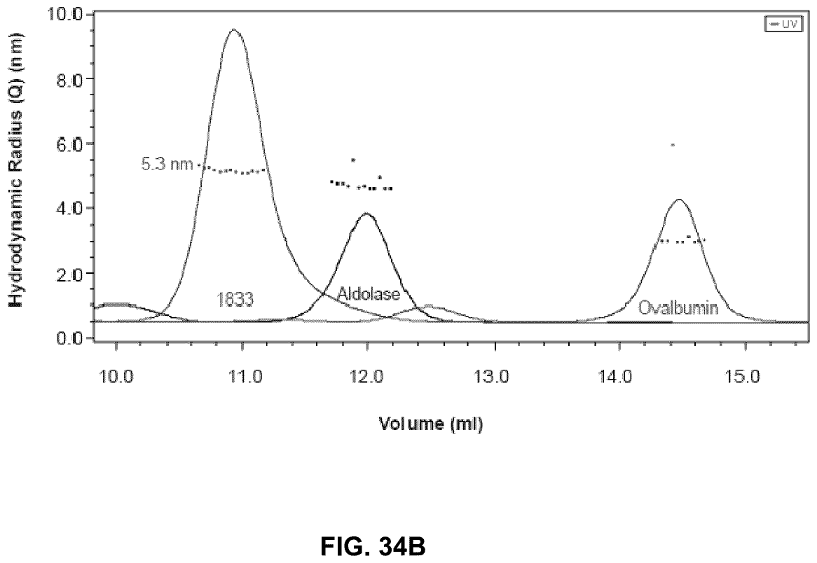

FIG. 34A-FIG. 34B presents characterization of Construct 1833 with size exclusion chromatography-light scattering (SEC-LS). FIG. 34A presents a molar mass distribution plot of Construct 1833 (in red) overlaid with two standards—Bovine Serum Albumin (BSA, MW=66 kDa) and Aldolase (MW=156 kDa). Construct 1833 was run at two concentrations, 0.59 mg/ml and a 20 fold dilution (0.08 mg/ml), revealing a polypeptide mass of 116 kDa and 121 kDa and sugar mass of 14 kDa and 13 kDa, respectively. The analysis yielded a total mass of the biologic at 132 and 134 kDa at the higher and lower concentration, indicated that Construct 1833 exists as a stable dimer in solution. FIG. 34B presents hydrodynamic radius distribution plots of Construct 1833 (red) overlaid with two standards—Aldolase (blue) and Ovalbumin (green). Construct 1833 eluted ahead of Aldolase, confirming a non-spherical/extended shape due to the presence of sugars, yielding a hydrodynamic radius (Rh) of 5.3 nm.

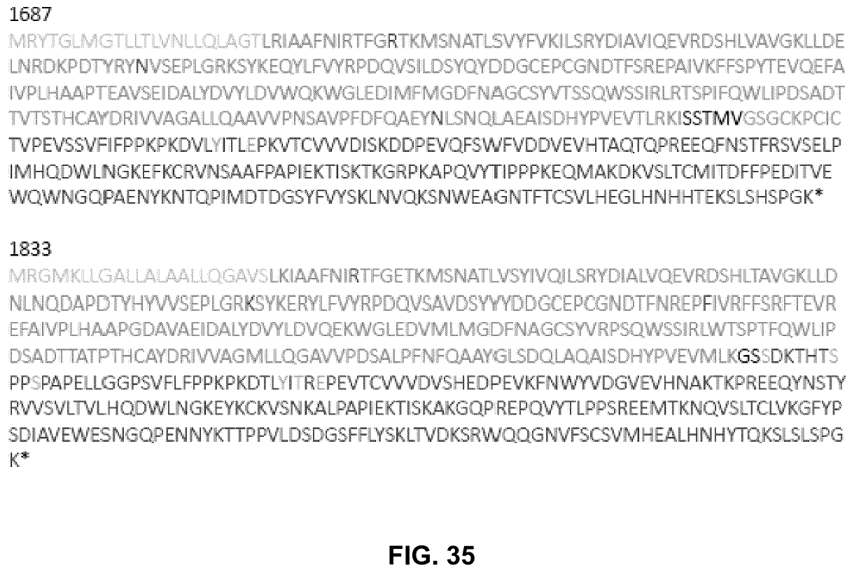

FIG. 35 presents amino acid sequences of a dual acting mouse Dnase1-Fc (Construct 1687) and the human DNASE1-Fc equivalent (Construct 1833). Highlighted features include a signal sequence, a DNAse1 platform sequence, a linker sequence, an IgG1 Fc sequence, mutations introduced into the DNAse backbone to confer DNASE1L3 activity and reduce acting binding, and mutations introduced into the IgG1 Fc domains to enhance FcRN endosomal recycling. In particular with regard to FIG. 35, gold font: Signal sequence; green font: DNASE1 platform sequence; black font: linker sequence; purple font: IgG1 Fc domain; red font: mutations introduced into the DNASE backbone to confer DNASE1L3 activity and reduce acting binding; cyan font: mutations introduced into the IgG1 Fc domains to enhance FcRN endosomal recycling.

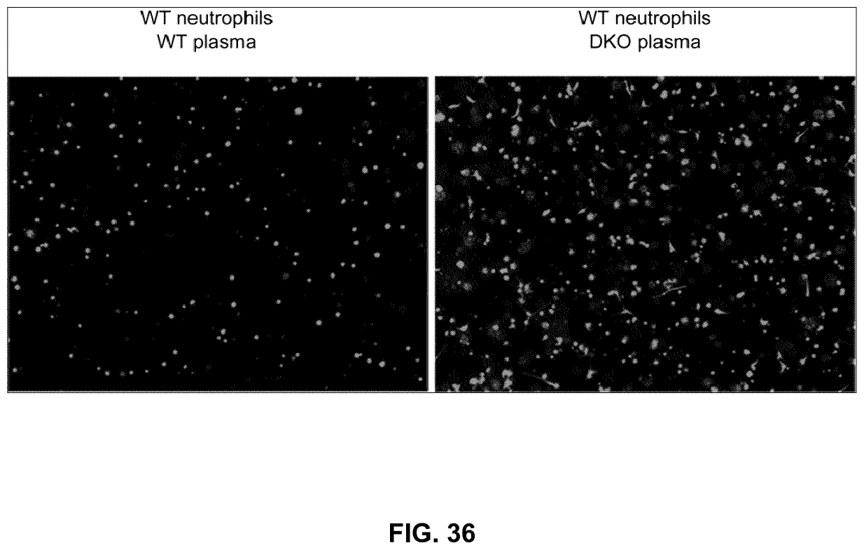

FIG. 36 presents images of data related to DKO mouse plasma induction of NETs in WT mouse neutrophils. Neutrophils from WT mice were isolated and plated into poly-lysine-coated wells of a 96-well plate and allowed to adhere at 37° C. in 100 ul RPMI. After 1 hr, the wells were spiked with 10 ul plasma from either a WT (left) or DKO (right) mouse and incubated for 2 hours. After adding Sytox Green (5 uM), images were captured on a fluorescent microscope. The WT neutrophils demonstrated increased fluorescence after being exposed to the plasma from a DKO mouse (right) compared to when the same neutrophils are exposed to the plasma of a WT mouse (left). Image captured at 40× magnification. It was found that plasma from DKO mice induced NETosis in WT murine neutrophils over and above the effects of plasma from WT mice.

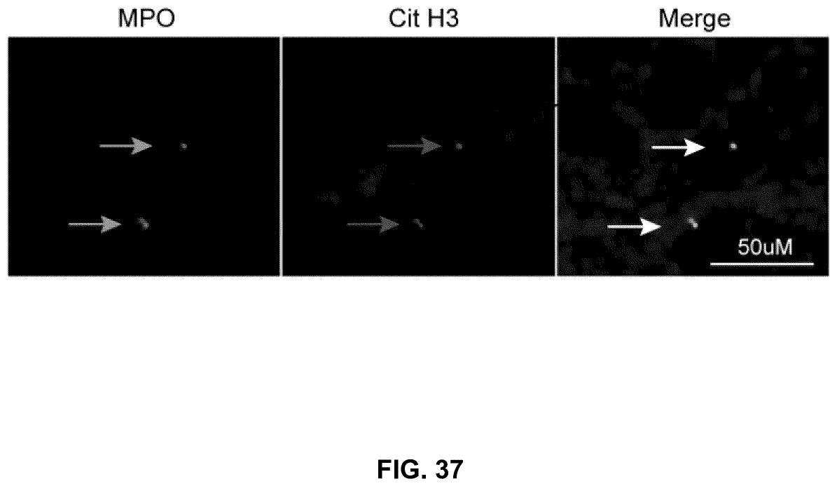

FIG. 37 presents immunofluorescence images related to immunofluorescence from a lung section of an untreated mouse removed from the study due to severe DAH showing deposition of MPO (green arrows, left panel) and Citrullinated Histone H3 (Cit-H3) (red arrows, middle panel) in the alveolar walls and DAPI stained nuclei in blue. Scale bar=50 uM. Mice which expired from DAH all revealed similar staining.

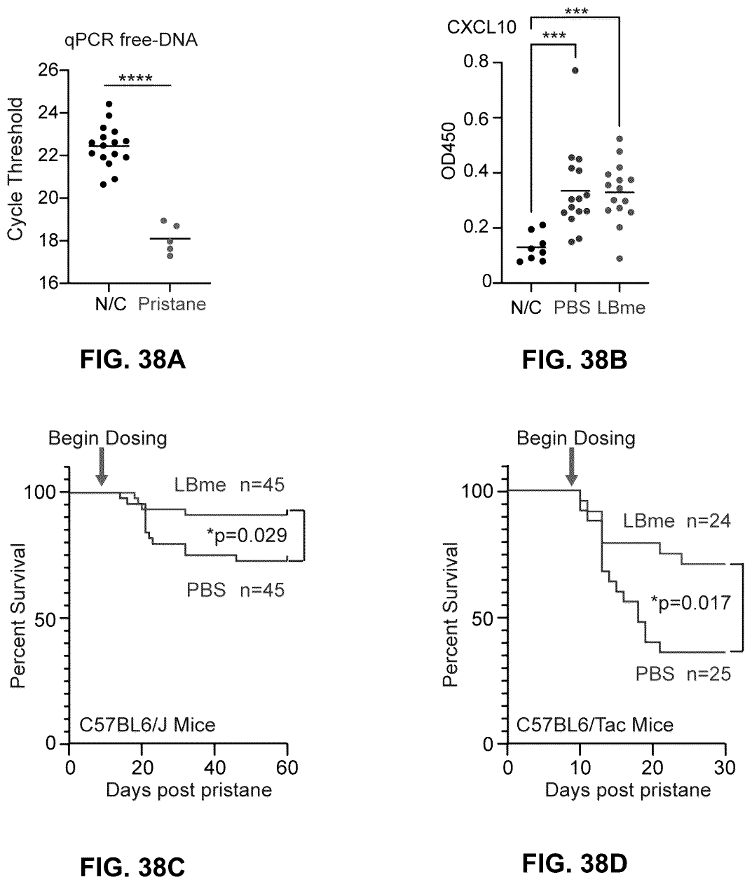

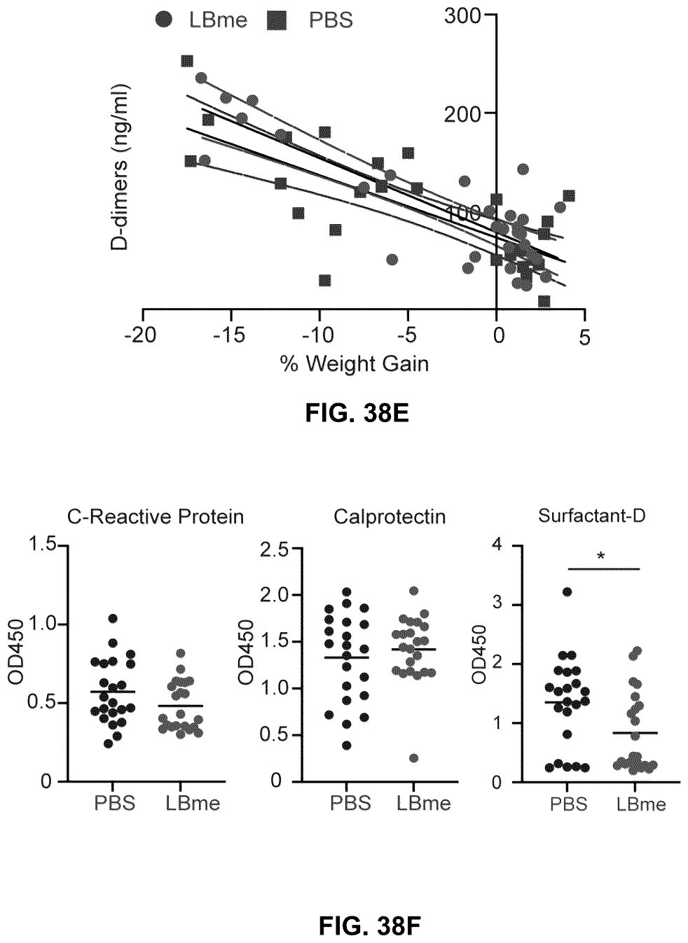

FIG. 38A-FIG. 38F present data and results related to the efficacy of Lbme (Construct 1687) in Diffuse Alveolar Hemorrhage in two strains of C57BL/6 mice. FIG. 38A demonstrates that WT mice had higher amounts of plasma cfDNA 14 days after pristane injection than untreated negative control N/C mice when analyzed by pPCR. ****p<0.0001, Student's two-tailed unpaired T-test. FIG. 38B presents data demonstrating that ten days after pristane injection, before the dosing strategy began, both cohorts of mice showed elevated levels of CXCL10 compared with (N/C) mice that did not receive pristane. FIG. 38C presents data and results related to C57BL/6J mice (The Jackson Laboratory) that were dosed i.p. with 500 ml of pristane on day zero, and weekly with either PBS or Lbme (Construct 1687) (1 mg/kg) following the first death of an animal post-pristane challenge (on day 10). The survival rate of dosed and vehicle treated animals was 95% vs 70%, respectively (p=0.029, Mantel-Cox). FIG. 38D presents data and results related to an identical study performed in C57BL/6Tac mice (Taconic Biosciences) with dosing beginning on day 9 post pristane, yielding a survival rate of 70% and 35%, for dosed and vehicle-treated animals respectively (p=0.017, Mantel-Cox). FIG. 38E demonstrates that D-dimers measured in C57BL/6J mice at 14 days inversely correlated with weight gain in the vehicle (slope=−6.35, R²=0.55, F=33.01, p<0.0001) and LBme-treated (slope=−7.54, R²=0.69, F=63.84, p<0.0001) cohorts. FIG. 38F demonstrates that C-reactive protein and calprotectin were equivalent in the treated and untreated cohorts, but surfactant-D levels were significantly higher in vehicle treated C57BL/6 mice at 14 days after pristane challenge, demonstrating that although acute-phase reaction and NETosis was equivalent in treated and untreated cohorts, Lbme (Construct 1687) reduced alveolar damage in the treated mice. *p<0.05, Student's two-tailed unpaired T-test. The results demonstrated that administering Lbme (Construct 1687) to WT mice following the onset of respiratory distress and an initial fatality in the pristane model rescued animals from fatal pulmonary hemorrhage and death due to lung injury and a DIC-related coagulopathy.

### Efficacy of the Human Isoform of LBme (1833) in SLE Patient Plasma and Blood

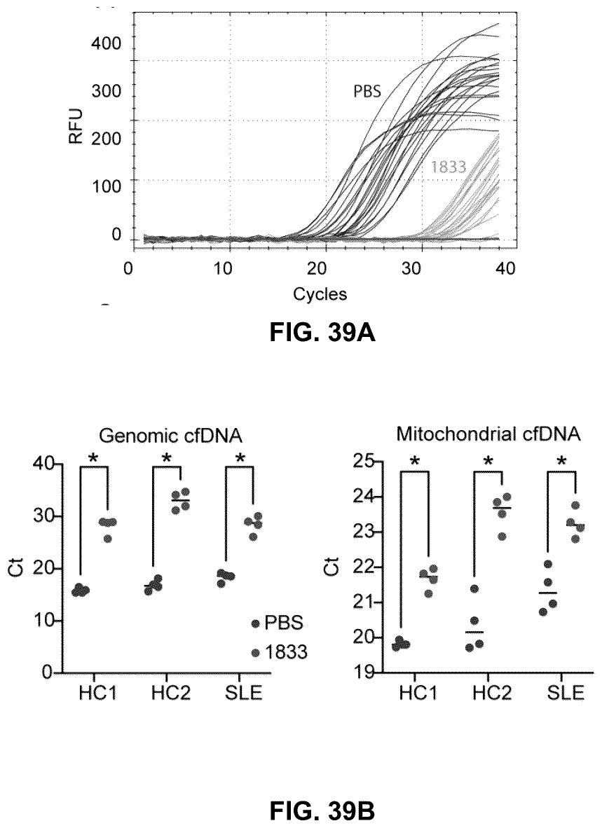

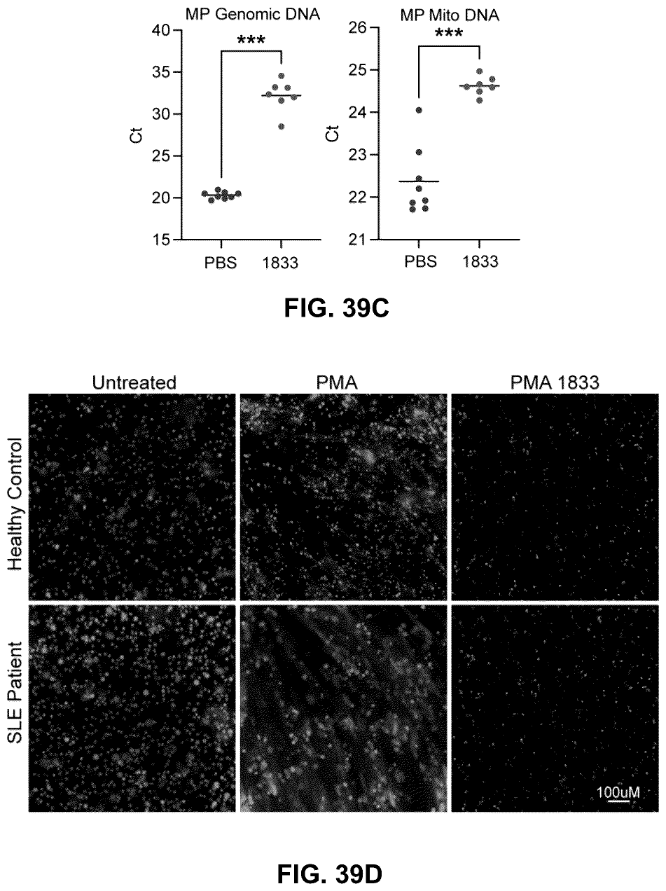

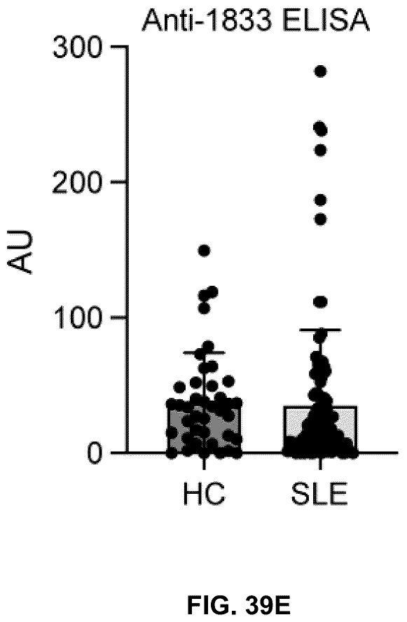

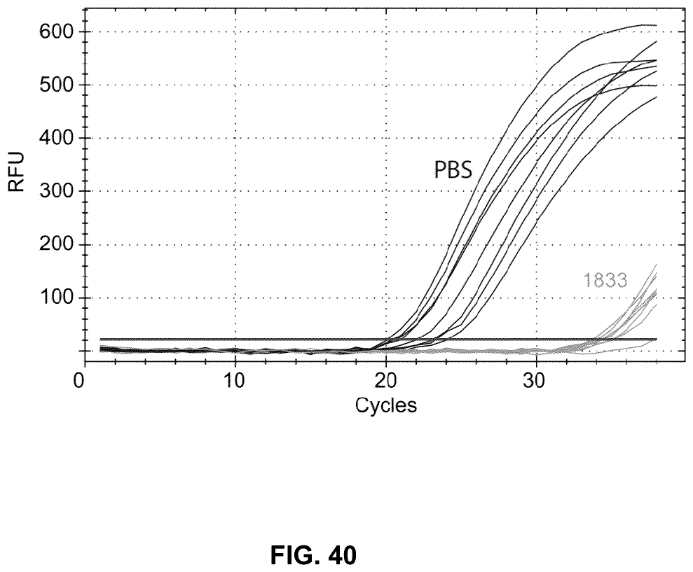

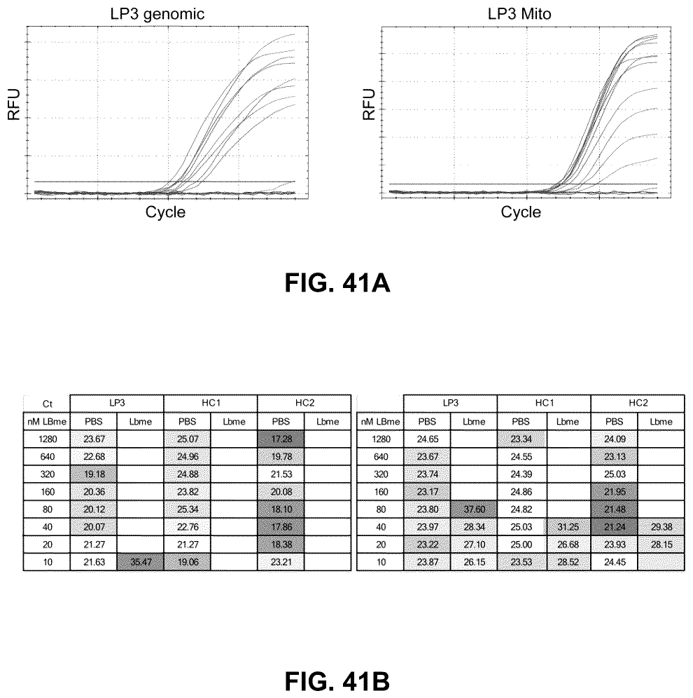

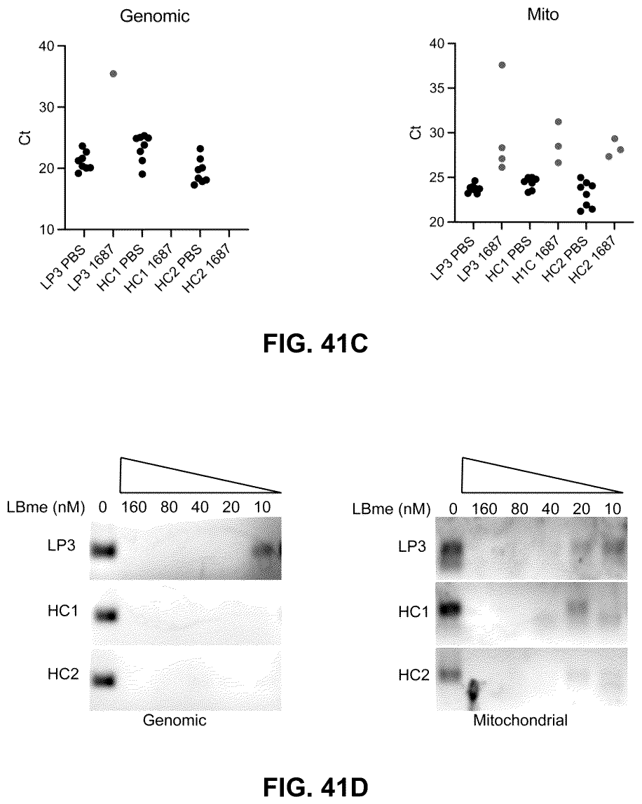

To demonstrate and characterize the effects of the human isoform of LBme (1833) in human lupus patients, the degradation of various types of cfDNA was quantified in the plasma of 4 human SLE patients and of 3 healthy controls (HC) following the addition of either PBS or the human isoform of LBme, called 1833, at the final concentration of 200 nM. It was found that 1833 rapidly degraded cell-free (cf) genomic DNA as demonstrated by the right shift of cycle threshold (Ct) values by qPCR (FIG. 39A). Similarly, when 1833 (200 nM) or PBS was added directly to whole blood of SLE patients, qPCR analysis of genomic cfDNA indicated no significant inhibition of 1833 in whole blood samples of SLE patients (FIG. 40). The degradation of cf-mitochondrial DNA (mtDNA) by 1833 was observed to be less robust than its effects on cf-genomic DNA, presumably due to the sequestration of mtDNA within the mitochondria (data not shown). To further study the effect of 1833 on cfDNA in SLE patients, whole blood from 2 HC and an SLE patient was treated with phorbol myristate acetate (PMA) for 2 hours at 37° C. Subsequently, either 1833 at a final concentration of 200 nM or PBS was added to the blood for the last 10 minutes of the incubation. qPCR quantitation of cf-genomic and cf-mtDNA revealed significantly higher Ct counts in the 1833-treated than the PBS-treated samples, demonstrating the efficient degradation of both cf-genomic and cf-mtDNA by 1833 (FIG. 39B). In separate experiments, Lbme (Construct 1687) was noted to degrade both genomic and mtDNA from human whole blood without the need to first stimulate with PMA (FIG. 41A-FIG. 41D). The effect of 1833 on microparticle-associated DNA (MP-DNA) in SLE was assessed by incubating MP-DNA (isolated from the whole blood of an SLE patient) for 10 minutes at 37° C. with 1833 at a final concentration of 200 nM or PBS and comparing the relative concentrations of MP-DNA by qPCR. It was found that 1833 efficiently degraded MP-DNA of both genomic and mitochondrial origin, as demonstrated by the increased Ct counts in the 1833-treated samples (FIG. 39C). Lastly, leukocytes from HC and SLE patients were isolated and NET formation was stimulated with PMA, demonstrating more robust NETosis in the SLE leukocytes (FIG. 39D). Incubating these samples with 1833 at a final concentration of 50 nM for 10 minutes demonstrated the efficient degradation of the PMA-induced NETs derived from leukocytes in both HS and SLE patients (FIG. 39D). Finally, to confirm that 1833 would not be targeted by autoantibodies in SLE patients, 1833 was incubated in the plasma of 40 HC and 99 SLE patients, 33 of whom possessed anti-DNASE1L3 autoantibodies, and no significant difference was found between the reactivity of 1833 to both groups (FIG. 39E). In other words, the plasma from 8 of the 99 SLE patients reacted with 1833, as compared to that from 3 of the 33 HCs, or 8% SLE reactivity vs. 10% HC reactivity. Of the 33 SLE patients with anti-DNASE1L3 Abs, 3 reacted to 1833 at levels comparable to reactivity observed in the HCs, clearly demonstrating that the antibodies targeting 1833 were not DNAse1L3 specific. Moreover, as 63% of SLE patients are positive for anti-DNASE1 Abs compared to 8% of HCs, the results indicated that DNASE1 enzyme backbone of 1833 was not reactive to anti-DNASE1 Abs in SLE patient plasma.

### ENUMERATED EMBODIMENTS

The following exemplary embodiments are provided, the numbering of which is not to be construed as designating levels of importance:
Embodiment 1: A method of treating, ameliorating, and/or preventing Diffuse Alveolar Hemorrhage (DAH) in a subject, the method comprising administering to the subject a therapeutically effective amount of a construct comprising the amino acid sequence:

Y-X1-LINKER-Fc-X2 (I), wherein:
Y is a human DNAse1 polypeptide or a human DNAse1L3 polypeptide;
X1 is a covalent bond, or X1 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1-IgG4.
Embodiment 2: The method of Embodiment 1, wherein the subject suffers from, is afflicted by, and/or has undergone at least one of the following conditions/treatments/procedures: ANCA vasculitis, Goodpasture's disease, (systemic) lupus erythematosus (SLE), antiphospholipid syndrome, Lane-Hamilton syndrome, post-sepsis, bone marrow and/or organ transplant, exposure to cytotoxic and non-cytotoxic drugs, acute kidney injury, viral and/or bacterial infection, poststreptococcal glomerulonephritis or bacterial endocarditis, and/or SARS-CoV-2 infection (COVID-19).
Embodiment 3: The method of any one of Embodiments 1-2, wherein the administering reduces, minimizes, and/or prevents DAH morbidity in the subject.
Embodiment 4: The method of any one of Embodiments 1-3, wherein the administering reduces, minimizes, and/or prevents development of acute severe respiratory distress syndrome (ARDS) in the subject.
Embodiment 5: The method of any one of Embodiments 1-4, wherein the administering reduces, minimizes, and/or prevents mortality due to DAH.
Embodiment 6: The method of any one of Embodiments 1-5, wherein the subject requires intensive care unit (ICU) treatment and wherein the administering reduces ICU time for the subject.
Embodiment 7: The method of any one of Embodiments 1-6, wherein the Fc comprises the amino acid sequence of SEQ ID NO:4 or SEQ ID NO:45.
Embodiment 8: The method of Embodiment 7, wherein at least one of C6 and C9 with respect to SEQ ID NO:4, or at least one of C1, C7, and C10 with respect to SEQ ID NO:45, is independently mutated to Gly or Ser.
Embodiment 9: The method of any one of Embodiments 7-8, wherein each one of C6 and C9 with respect to SEQ ID NO:4, or each one of C1, C6, and C9 with respect to SEQ ID NO:45, is independently mutated to Gly or Ser.
Embodiment 10: The method of any one of Embodiments 7-9, wherein the construct comprises at least one of the following mutations with respect to SEQ ID NO:4: M32Y, S34T, T36E, or wherein the construct comprises at least one of the following mutations with respect to SEQ ID NO:45: M33Y, S35T, T37E.
Embodiment 11: The method of any one of Embodiments 7-10, wherein the construct comprises each one of the following mutations with respect to SEQ ID NO:4: M32Y, S34T, T36E or wherein the construct comprises each one of the following mutations with respect to SEQ ID NO:45: M33Y, S35T, T37E.
Embodiment 12: The method of any one of Embodiments 1-11, wherein the LINKER is a chemical bond or absent.
Embodiment 13: The method of any one of Embodiments 1-11, wherein the LINKER is a polypeptide comprising 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, and/or 1 amino acid(s).
Embodiment 14: The method of any one of Embodiments 1-11 and 13, wherein the LINKER comprises A, C, D, E, F, G, H, I, K, L, M, N, P, Q, R, S, T, V, Y, W, GS, SG, GG, SS, GGG, SGG, GSG, GGS, GSS, SGS, SSG, SSS, and/or GSC.
Embodiment 15: The method of any one of Embodiments 1-11 and 13-14, wherein the LINKER comprises GGGGSGGGGS (SEQ ID NO:5), SSTMVRS (SEQ ID NO:40), and/or SSTMVGS (SEQ ID NO:41).
Embodiment 16: The method of any one of Embodiments 1-11 and 13-15, wherein the LINKER comprises ELKTPLGDTTHTXPRZPAPELLGGP (SEQ ID NO:6), wherein each occurrence of X is C, G, or S, and wherein each occurrence of Z is C, G, or S.
Embodiment 17: The method of any one of Embodiments 1-16, wherein X1 is a covalent bond.
Embodiment 18: The method of any one of Embodiments 1-16, wherein X1 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof.
Embodiment 19: The method of any one of Embodiments 1-18, wherein X2 is a covalent bond.
Embodiment 20: The method of any one of Embodiments 1-18, wherein X2 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof.
Embodiment 21: The method of any one of Embodiments 1-20, wherein the DNAse1 lacks at least a portion of residues 1-22 corresponding to SEQ ID NO:1.
Embodiment 22: The method of any one of Embodiments 1-21, wherein the DNAse1 lacks residues 1-22 corresponding to SEQ ID NO:1.
Embodiment 23: The method of any one of Embodiments 1-22, wherein the DNAse1 comprises at least one of the following mutations with respect to SEQ ID NO:1: Q31R, E35R, Y46H, Y46S, V88N, N96K, D109N, V111T, A136F, R148S, E149N, M186I, L208P, D220N, R244Q, D250N, A252T, G262N, D265N, and L267T.
Embodiment 24: The method of any one of Embodiments 1-23, wherein the Fc comprises at least one of the following mutations with respect to SEQ ID NO:4: C6G, C6S, C9G, C9S, M32Y, S34T, and T36E, or wherein the Fc comprises at least one of the following mutations with respect to SEQ ID NO:45: C1G, C1S, C7G, C7S, C10G, C10S, M33Y, S35T, and T37E.
Embodiment 25: The method of any one of Embodiments 1-24, wherein the construct comprises the amino acid sequence selected from the group consisting of SEQ ID NOs:7-17, 44, and 46-53.
Embodiment 26: The method of any one of Embodiments 1-20, wherein the DNAse1L3 lacks at least one of the following: residues 291-305 of SEQ ID NO:2; residues 296-304 of SEQ ID NO:2; residues 292-304 of SEQ ID NO:2; residues A-B of SEQ ID NO:2, wherein A ranges from 291 to 296 and B ranges from 304 to 305.
Embodiment 27: The method of any one of Embodiments 1-20 and 26, wherein the DNAse1L3 comprises at least one of the following mutations with respect to SEQ ID NO:2: E33R, M42T, V44H, V88T, N96K, A127N, V129T, K147S, D148N, L207P, D219N, and V254T.
Embodiment 28: The method of any one of Embodiments 1-11 and 26-27, wherein the Fc comprises at least one of the following mutations with respect to SEQ ID NO:4: C6G, C6S, C9G, C9S, M32Y, S34T, and T36E, or wherein the Fc comprises at least one of the following mutations with respect to SEQ ID NO:45: C1G, C1S, C7G, C7S, C10G, C10S, M33Y, S35T, and T37E.
Embodiment 29: The method of any one of Embodiments 1-20 and 26-28, wherein the construct comprises the amino acid sequence selected from the group consisting of SEQ ID NOs:18-28.
Embodiment 30: The method of any one of Embodiments 1-29, wherein the construct is expressed in a mammalian cell.
Embodiment 31: The method of Embodiment 30, wherein the mammalian cell is stably transfected with human ST6 beta-galatosamide alpha-2,6-sialyltransferase (also known as ST6GAL1).
Embodiment 32: The method of any one of Embodiments 30-31, wherein the mammalian cell is grown in a cell culture supplemented with sialic acid and/or N-acetylmannosamine (also known as 1,3,4-O-Bu₃ManNAc).
Embodiment 33: The method of any one of Embodiments 1-32, wherein the construct is soluble.
Embodiment 34: The method of any one of Embodiments 1-33, wherein the construct is a secreted product of a DNAse1 and/or DNAse1L3 precursor construct expressed in a mammalian cell, wherein the DNAse1 and/or DNAse1L3 precursor construct comprises a signal peptide sequence and a DNAse1 and/or DNAse1L3 polypeptide, wherein the DNAse1 and/or DNAse1L3 precursor construct undergoes proteolytic processing to yield the DNAse1 and/or DNAse1L3 construct.
Embodiment 35: The method of any one of Embodiments 1-34, wherein in the DNAse1 and/or DNAse1L3 precursor construct the signal peptide sequence is conjugated to the N-terminus of the DNAse1 and/or DNAse1L3 polypeptide.
Embodiment 36: The method of any one of Embodiments 1-35, wherein the construct is administered acutely or chronically to the subject.
Embodiment 37: The method of any one of Embodiments 1-36, wherein the construct is administered locally, regionally, parenterally, or systemically to the subject.
Embodiment 38: The method of any one of Embodiments 1-37, wherein the construct, or its precursor construct, is delivered on an encoded vector to the subject, wherein the vector encodes the construct or precursor construct, which is transcribed and translated from the vector upon administration of the vector to the subject.
Embodiment 39: The method of any one of Embodiments 1-38, wherein the construct is administered to the subject by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical.
Embodiment 40: The method of any one of Embodiments 1-39, wherein the construct is administered to the subject as a pharmaceutical composition further comprising at least one pharmaceutically acceptable carrier.
Embodiment 41: The method of any one of Embodiments 1-40, wherein the construct comprises at least one of the following:
(a) a homodimeric construct comprising two independently selected constructs (I), wherein each Y is an independently selected human DNAse1 polypeptide;
(b) a homodimeric construct comprising two independently selected constructs (I), wherein each Y is an independently selected human DNAse1L3 polypeptide; and/or
(c) a heterodimeric construct comprising two independently selected constructs (I), wherein the Y in one of the two (I) is a human DNAse1 polypeptide and the Y in the other (I) is a human DNAse1L3 polypeptide.
Embodiment 42: The method of any one of Embodiments 1-41, wherein the subject is a mammal.
Embodiment 43: The method of Embodiment 42, wherein the mammal is human.
Embodiment 44. A construct comprising the amino acid sequence: DNAse1-LINKER-Fc (I)
wherein: DNAse 1 is a human DNAse1 polypeptide; LINKER is a polypeptide comprising 1-100 amino acids; Fc is the Fc domain of human IgG1; wherein the construct comprises the amino acid sequence of any one of SEQ ID NOs:44 and 46-53.
Embodiment 45: A construct comprising the amino acid sequence: DNAse1-LINKER-Fc (I), wherein: DNAse 1 is a human DNAse1 polypeptide or a functional variant thereof, wherein the DNase1 polypeptide comprises a modification for decreased actin binding activity, and wherein the DNAse1 polypeptide further comprises an azurocidin signal sequence; LINKER is a polypeptide comprising 1-100 amino acids; and Fc is a human immunoglobulin Fc domain, wherein the Fc domain comprises one or more amino acid residue modifications for enhanced endosomal recycling relative to a human immunoglobulin Fc domain.
Embodiment 46. The construct of Embodiment 44 or 45, wherein the construct is expressed in a mammalian cell.
Embodiment 47. The construct of Embodiment 46, wherein the mammalian cell is stably transfected with human ST6 beta-galatosamide alpha-2,6-sialyltransferase (also known as ST6GAL1).
Embodiment 48. The construct of any one of Embodiments 46-47, wherein the mammalian cell is grown in a cell culture supplemented with sialic acid and/or N-acetylmannosamine (also known as 1,3,4-O-Bu3ManNAc).
Embodiment 49. The construct of any one of Embodiments 44-48, wherein the construct is soluble.
Embodiment 50. The construct of any one of Embodiments 44-49, wherein the construct is a secreted product of a DNAse1 precursor construct expressed in a mammalian cell, wherein the DNAse1 precursor construct comprises a signal peptide sequence and a DNAse1 polypeptide, wherein the DNAse1 precursor construct undergoes proteolytic processing to yield the DNAse1 construct.
Embodiment 51. The construct of Embodiment 50, wherein in the DNAse1 precursor construct the signal peptide sequence is conjugated to the N-terminus of the DNAse1 polypeptide.
Embodiment 52. A method of treating, ameliorating, and/or preventing forms of lupus associated with DNAse1 and/or DNAse1L3 deficiency in a subject, the method comprising administering to the subject a therapeutically effective amount of a construct of any one of Embodiments 44-51.
Embodiment 53. The method of Embodiment 52, wherein the lupus comprises systemic lupus erythematosus (SLE).
Embodiment 54. A method of treating, ameliorating, and/or preventing diseases and/or disorders associated with inefficient NET hydrolysis (NETolysis) in a subject, the method comprising administering to the subject a therapeutically effective amount of a construct of any one of Embodiments 44-51.
Embodiment 55. A method of treating, ameliorating, and/or preventing an autoimmune disorder associated with DNAse1 and/or DNAse1L3 deficiency in a subject, the method comprising administering to the subject a therapeutically effective amount of a construct of any one of Embodiments 44-51.
Embodiment 56. The method of Embodiment 55, wherein the autoimmune disorder comprises lupus, thyroid autoimmune disease, and/or Hypocomplementeric Urticarial Vasculitis Syndrome (HUVS).
Embodiment 57. A method of treating, ameliorating, and/or preventing pathologic thrombosis in a subject, the method comprising administering to the subject a therapeutically effective amount of a construct of any one of Embodiments 44-51.
Embodiment 58. The method of Embodiment 57, wherein the pathologic thrombosis comprises microvascular thrombosis, venous thrombosis, and/or arterial thrombosis.
Embodiment 59. The method of Embodiment 57, wherein the pathologic thrombosis leads to stroke and/or makes the subject susceptible to stroke.
Embodiment 60. The method of Embodiment 57, wherein the pathologic thrombosis comprises neutrophilic thrombosis.
Embodiment 61. The method of Embodiment 60, wherein the neutrophilic thrombosis comprises at least one of Anti-Neutrophilic Cytoplasmic Autoantibodies (ANCA) vasculitis, Thrombotic thrombocytopenic purpura (TTP), and Bechet's (or Behcet's) disease or syndrome.
Embodiment 62. A method of treating, ameliorating, and/or preventing a myocardial infarction in a subject, the method comprising administering to the subject a therapeutically effective amount of a construct of any one of Embodiments 44-51.
Embodiment 63. A method of treating, ameliorating, and/or preventing cancer metastasis in a subject, the method comprising administering to the subject a therapeutically effective amount of a construct of any one of Embodiments 44-51.
Embodiment 64. A method of treating, ameliorating, and/or preventing inefficient NET hydrolysis (“NETolysis”) in a subject afflicted with a bacterial and/or viral infection, the method comprising administering to the subject a therapeutically effective amount of a construct of any one of Embodiments 44-51.
Embodiment 65. A method of treating, ameliorating, and/or preventing systemic inflammation, organ damage, and/or sepsis in a subject afflicted with a bacterial and/or viral infection, the method comprising administering to the subject a therapeutically effective amount of a construct of any one of Embodiments 44-51.
Embodiment 66. A method of treating, ameliorating, and/or preventing pathologic thrombosis in a subject afflicted with a bacterial and/or viral infection, the method comprising administering to the subject a therapeutically effective amount of a construct of any one of Embodiments 44-51.
Embodiment 67. The method of any one of Embodiments 64-66, wherein the virus is a coronavirus.
Embodiment 68. The method of Embodiment 67, wherein the coronavirus is SARS-Cov and/or SARS-Cov-2.
Embodiment 69. The method of any one of Embodiments 66-68, wherein the thrombosis leads to stroke and/or makes the subject susceptible to stroke.
Embodiment 70. A method of treating, ameliorating, and/or preventing lung injury in a subject afflicted with acute kidney injury (AKI), the method comprising administering to the subject a therapeutically effective amount of a construct of any one of Embodiments 44-51.
Embodiment 71. The method of Embodiment 70, wherein the lung injury is acute lung injury (AKI).
Embodiment 72. The method of Embodiment 71, wherein the AKI comprises at least one of non-cardiogenic pulmonary edema, impaired lung fluid clearance, and respiratory failure requiring mechanical ventilation.
Embodiment 73. A method of reducing, reversing increase of, and/or suppressing levels of IL-6 in a subject afflicted with acute kidney injury (AKI), the method comprising administering to the subject a therapeutically effective amount of a construct of any one of Embodiments 44-51.
Embodiment 74. A method of treating, ameliorating, and/or preventing Diffuse Alveolar Hemorrhage (DAH) in a subject, the method comprising administering to the subject a therapeutically effective amount of a construct of any one of Embodiments 44-51.
Embodiment 75. The method of Embodiment 74, wherein the subject suffers from, is afflicted by, and/or has undergone at least one of the following conditions/treatments/procedures: ANCA vasculitis, Goodpasture's disease, (systemic) lupus erythematosus (SLE), antiphospholipid syndrome, Lane-Hamilton syndrome, post-sepsis, bone marrow and/or organ transplant, exposure to cytotoxic and non-cytotoxic drugs, acute kidney injury, viral and/or bacterial infection, poststreptococcal glomerulonephritis or bacterial endocarditis, and/or SARS-CoV-2 infection (COVID-19).
Embodiment 76. The method of any one of Embodiments 74-75, wherein the administering reduces, minimizes, and/or prevents DAH morbidity in the subject.
Embodiment 77. The method of any one of Embodiments 74-76, wherein the administering reduces, minimizes, and/or prevents development of acute severe respiratory distress syndrome (ARDS) in the subject.
Embodiment 78. The method of any one of Embodiments 74-77, wherein the administering reduces, minimizes, and/or prevents mortality due to DAH.
Embodiment 79. The method of any one of Embodiments 74-78, wherein the subject requires intensive care unit (ICU) treatment and wherein the administering reduces ICU time for the subject.
Embodiment 80. A method of treating, ameliorating, and/or preventing Aicardi-Goutieres Syndrome (AGS) in a subject, the method comprising administering to the subject a therapeutically effective amount of a construct of any one of Embodiments 44-51.
Embodiment 81. The method of Embodiment 80, wherein the administering minimizes, reduces, and/or prevents mortality due to AGS.
Embodiment 82. The method of any one of Embodiments 80-81, wherein the AGS-afflicted subject has “classic” AGS with prenatal or infantile onset.
Embodiment 83. The method of any one of Embodiments 80-81, wherein the AGS-afflicted subject has subacute onset of profound neurological regression, a variable combination of spasticity and dystonia associated with normal neuroimaging, and/or specific white matter changes and/or intracranial calcification.
Embodiment 84. The method of any one of claims 80-81, wherein the AGS-afflicted subject has bilateral striatal necrosis.
Embodiment 85. The method of any one of Embodiments 80-81, wherein the AGS-afflicted subject has progressive (‘non-syndromic’) spastic paraparesis confined to at least one lower limb.
Embodiment 86. The method of any one of Embodiments 80-81, wherein the AGS-afflicted subject has intracerebral, large vessel disease.
Embodiment 87. The method of any one of Embodiments 80-81, wherein the AGS-afflicted subject has chilblain like lesions of the skin.
Embodiment 88. The method of any one of Embodiments 52-87, wherein the construct is administered acutely or chronically to the subject.
Embodiment 89. The method of any one of Embodiments 52-88, wherein the construct is administered locally, regionally, parenterally, and/or systemically to the subject.
Embodiment 90. The method of any one of Embodiments 52-89, wherein the construct, and/or its precursor construct, is delivered on an encoded vector to the subject, wherein the vector encodes the construct or precursor construct, which is transcribed and translated from the vector upon administration of the vector to the subject.
Embodiment 91. The method of any one of Embodiments 52-90, wherein the construct is administered to the subject by at least one route selected from the group consisting of subcutaneous, oral, aerosol, inhalational, rectal, vaginal, transdermal, subcutaneous, intranasal, buccal, sublingual, parenteral, intrathecal, intragastrical, ophthalmic, pulmonary, and topical.
Embodiment 92. The method of any one of Embodiments 52-91, wherein the construct is administered to the subject as a pharmaceutical composition further comprising at least one pharmaceutically acceptable carrier.
Embodiment 93. The method of any one of Embodiments 52-92, wherein the subject is a mammal.
Embodiment 94. The method of Embodiment 93, wherein the mammal is human.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1-51. (canceled)

52. A method of treating, ameliorating, or preventing Diffuse Alveolar Hemorrhage (DAH) in a subject, the method comprising administering to the subject a therapeutically effective amount of a construct comprising the amino acid sequence: 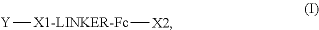
wherein:
Y is a human DNAse1 polypeptide or a human DNAse1L3 polypeptide;
X1 is a covalent bond, or X1 is the peptide of amino acid sequence RAFTNNRKSVSLKKRKKGNRS (SEQ ID NO:3), RAFTNNRKSVSLKKRKKGNRSGGVKKPHRYRPGTVAL (SEQ ID NO:54), KQTARKAPRKQLATKAARKSAPATGGVKKPHRYRPGTVAL (SEQ ID NO:55), or a fragment thereof;
LINKER is a chemical bond or a polypeptide comprising 1-100 amino acids;
X2 is null, or X2 is the peptide of amino acid sequence RAFTNNRKSVSLKKRKKGNRS (SEQ ID NO:3) or a fragment thereof;
Fc is the Fc domain of human IgG1-IgG4.

53. The method of claim 52, wherein the subject suffers from, is afflicted by, or has undergone at least one of the following conditions/treatments/procedures:
ANCA vasculitis, Goodpasture's disease, (systemic) lupus erythematosus (SLE), antiphospholipid syndrome, Lane-Hamilton syndrome, post-sepsis, bone marrow or organ transplant, exposure to cytotoxic or non-cytotoxic drugs, acute kidney injury, viral or bacterial infection, poststreptococcal glomerulonephritis or bacterial endocarditis, or SARS-CoV-2 infection (COVID-19).

54. The method of claim 52, wherein the administering reduces, minimizes, or prevents DAH morbidity in the subject.

55. The method of claim 52, wherein the administering reduces, minimizes, or prevents development of acute severe respiratory distress syndrome (ARDS) in the subject.

56. The method of claim 52, wherein the administering reduces, minimizes, or prevents mortality due to DAH.

57. The method of claim 52, wherein the subject requires intensive care unit (ICU) treatment and wherein the administering reduces ICU time for the subject.

58. (canceled)

59. The method of claim 52, wherein at least one of the following applies:
(a) at least one of C6 and C9 with respect to SEQ ID NO:4, or at least one of C1, C7, and C10 with respect to SEQ ID NO:45, is independently mutated to Gly or Ser;
(b) each one of C6 and C9 of the Fe with respect to SEQ ID NO: 4, or each one of C1, C7, and C10 with respect to SEQ ID NO:45, is independently mutated to Gly or Ser;
(c) the construct comprises at least one of the following mutations with respect to SEQ ID NO:4: M32Y, S34T, T36E or wherein the construct comprises at least one of the following mutations with respect to SEQ ID NO: 45: M33Y, S35T, T37E;
(d) the construct comprises each one of the following mutations with respect to SEQ ID NO:4: M32Y, S34T, T36E or wherein the construct comprises each one of the following mutations with respect to SEQ ID NO:45: M33Y, S35T, T37E.

60-62. (canceled)

63. The method of claim 52, wherein at least one of the following applies:
(a) the LINKER is a chemical bond or absent;
(b) the LINKER is a polypeptide comprising 1-100, 1-90, 1-80, 1-70, 1-60, 1-50, 1-40, 1-30, 1-20, 1-10, 1-9, 1-8, 1-7, 1-6, 1-5, 1-4, 1-3, 1-2, and/or 1 amino acid(s);
(c) the LINKER comprises A, C, D, E, F, G, H, I, K, L, M, N, P, Q, R, S, T, V, Y, W, GS, SG, GG, SS, GGG, SGG, GSG, GGS, GSS, SGS, SSG, SSS, and/or GSC;
(d) the LINKER comprises GGGGSGGGGS (SEQ ID NO:5), SSTMVRS (SEQ ID NO:40), or SSTMVGS (SEQ ID NO:41);
(e) the LINKER comprises ELKTPLGDTTHTXPRZPAPELLGGP (SEQ ID NO:6), wherein each occurrence of X is C, G, or S, and wherein each occurrence of Z is C, G, or S.

64-69. (canceled)

70. The method of claim 52, wherein one of the following applies:
(a) X2 is a covalent bond;
(b) X2 is the peptide of amino acid sequence
RAFTNNRKSVSLKKRKKGNRS (SEQ ID NO:3),
RAFTNNRKSVSLKKRKKGNRSGGVKKPHRYRPGTVAL (SEQ ID NO:54),
KQTARKAPRKQLATKAARKSAPATGGVKKPHRYRPGTVAL (SEQ ID NO:55),
or a fragment thereof.

71. (canceled)

72. The method of claim 52, wherein the DNAse1 lacks at least a portion of residues 1-22 corresponding to SEQ ID NO:1.

73. The method of claim 52, wherein the DNAse1 lacks residues 1-22 corresponding to SEQ ID NO:1.

74. The method of claim 52, wherein the DNAse1 comprises at least one of the following mutations with respect to SEQ ID NO:1: Q31R, E3SR, Y46H, Y46S, V88N, N96K, D109N, V111T, A136F, R148S, E149N, M186I, L208P, D220N, R244Q, D250N, A252T, G262N, D265N, and L267T.

75. The method of claim 52, wherein the Fc comprises at least one of the following mutations with respect to SEQ ID NO:4: C6G, C6S, C9G, C9S, M32Y, S34T, and T36E, or wherein the Fc comprises at least one of the following mutations with respect to SEQ ID NO:45: C1G, C1S, C7G, C7S, C10G, C10S, M33Y, S35T, and T37E.

76. The method of claim 52, wherein the construct comprises the amino acid sequence selected from the group consisting of SEQ ID NOs:7-17, 44, and 46-53.

77. The method of claim 52, wherein at least one of the following applies:
(a) the DNAse1L3 lacks at least one of the following: residues 291-305 of SEQ ID NO:2; residues 296-304 of SEQ ID NO:2; residues 292-304 of SEQ ID NO:2; residues A-B of SEQ ID NO:2, wherein A ranges from 291 to 296 and B ranges from 304 to 305;
(b) the DNAse1L3 comprises at least one of the following mutations with respect to SEQ ID NO:2: E33R, M42T, V44H, V88T, N96K, A127N, V129T, K147S, D148N, L207P, D219N, and V254T.

78. (canceled)

79. The method of claim 52, wherein the Fc comprises at least one of the following mutations with respect to SEQ ID NO:4: C6G, C6S, C9G, C9S, M32Y, S34T, and T36E, or wherein the Fc comprises at least one of the following mutations with respect to SEQ ID NO:45: C1G, C1S, C7G, C7S, C10G, C10S, M33Y, S35T, and T37E.

80. The method of claim 52, wherein the construct comprises the amino acid sequence selected from the group consisting of SEQ ID NOs:18-28.

81. The method of claim 52, wherein the construct is expressed in a mammalian cell.

82-83. (canceled)

84. The method of claim 52, wherein the construct is soluble.

85. The method of claim 52, wherein the construct is a secreted product of a DNAse1 or DNAse1L3 precursor construct expressed in a mammalian cell, wherein the DNAse1 or DNAse1L3 precursor construct comprises a signal peptide sequence and a DNAse1 or DNAse1L3 polypeptide, wherein the DNAse1 or DNAse1L3 precursor construct undergoes proteolytic processing to yield the DNAse1 or DNAse1L3 construct.

86. The method of claim 52, wherein in the DNAse1 or DNAse1L3 precursor construct the signal peptide sequence is conjugated to the N-terminus of the DNAse1 or DNAse1L3 polypeptide.

87. The method of claim 52, wherein the construct is administered intravenously to the subject, optionally wherein the construct is administered to the subject as a pharmaceutical composition further comprising at least one pharmaceutically acceptable carrier.

88-91. (canceled)

92. The method of claim 52, wherein the construct comprises at least one of the following:
(a) a homodimeric construct comprising two independently selected constructs (I), wherein each Y is an independently selected human DNAse1 polypeptide;
(b) a homodimeric construct comprising two independently selected constructs (I), wherein each Y is an independently selected human DNAse1L3 polypeptide; or
(c) a heterodimeric construct comprising two independently selected constructs (I), wherein the Y in one of the two (I) is a human DNAse1 polypeptide and the Y in the other (I) is a human DNAse1L3 polypeptide.

93. The method of claim 52, wherein the subject is a mammal, optionally wherein the mammal is human.

94. (canceled)
